(19) United States
(12) Patent Application Publication
(10) Pub. No.: US 2026/0355354 A1
(43) Pub. Date: Nov. 12, 2026

(54) CRISPR-BASED GENE EDITING PLATFORM WITH DNA ORIGAMI NANOROBOT SYSTEM ACROSS THE BLOOD-BRAIN BARRIER

(71) Applicant: Abraham Vargas, Torrance, CA (US)

(72) Inventor: Abraham Vargas, Torrance, CA (US)

(21) Appl. No.: 19/717,797

(22) Filed: Jun. 24, 2026

(51) Int. Cl. A61K 48/00; A61K 9/00; A61P 25/00

(52) U.S. Cl. CPC A61K 48/0058; A61K 9/0019; A61K 48/0041; A61K 48/0075; A61K 48/0083; A61K 48/0091; A61P 25/00

(57) ABSTRACT

A compartmentalized DNA origami nanorobot integrates three CRISPR gene-editing modalities into a single systemically deliverable platform for genetic neurological disorders. A self-assembled spherical DNA nanocage of about 100 to 140 nanometers encloses three discrete compartments that separately house a base editor, a prime editor, and a Cas13 RNA editor, preventing premature editor interaction. Transferrin-receptor aptamers and a polyethylene glycol coating enable intravenous administration and receptor-mediated transcytosis across the blood-brain barrier. Three sequential logic gates, each requiring at least two molecular inputs, coordinate temporally ordered release: base editing for permanent correction, prime editing for templated modification, and Cas13 for reversible messenger RNA regulation. In disorders such as Huntington disease, the coordinated multimodal action provides reduction of pathogenic protein burden through complementary permanent, templated, and reversible editing mechanisms.

### CROSS-REFERENCE TO RELATED APPLICATIONS

This application claims the benefit of and priority under 35 U.S.C. § 119(e) to U.S. Provisional Patent Application No. 63/959,950, filed Jan. 14, 2026, titled “CRISPR-Based Gene Editing Platform with DNA Origami Nanorobot System Across the BBB” (Confirmation No. 8186), the entire disclosure of which is incorporated herein by reference in its entirety.

### STATEMENT REGARDING FEDERALLY SPONSORED RESEARCH OR DEVELOPMENT

No part of this invention was made with government support. There is no federally sponsored research or development applicable to this invention.

### REFERENCE TO A SEQUENCE LISTING

Not applicable. No formal sequence listing under 37 C.F.R. §§ 1.831 through 1.835 is required because the specification does not disclose any nucleotide sequence of ten or more specifically defined nucleotides or any amino acid sequence of four or more specifically defined amino acids meeting the disclosure threshold of World Intellectual Property Organization Standard ST.26. Sequences referenced in the specification are described functionally and by reference to their biological targets and structural roles.

### BACKGROUND OF THE INVENTION

### Field of the Invention

The present invention relates generally to therapeutic delivery systems for gene editing, and more particularly to compartmentalized DNA nanostructures that integrate multiple distinct CRISPR-based gene-editing platforms within a single addressable vehicle. The invention concerns the field of nucleic-acid nanotechnology as applied to the controlled, spatially segregated, and temporally coordinated delivery of genome-editing and transcriptome-editing agents to target cells.

More specifically, the invention pertains to a compartmentalized DNA origami nanorobot system configured to carry a base editor, a prime editor, and a Cas13 RNA-targeting effector in physically discrete internal compartments, to traverse the blood-brain barrier following systemic administration, and to release each editing modality in a defined sequence within target cells of the central nervous system. The invention thereby relates to the convergence of three otherwise independent technical fields: CRISPR-based genome and transcriptome editing chemistry, structural DNA nanotechnology, and receptor-mediated transport across the blood-brain barrier.

The invention further relates to logic-gated sequential release of therapeutic cargo, wherein the conditional opening of internal compartments is governed by the coincident detection of multiple distinct molecular inputs. The invention is applicable to neurological and neurodegenerative conditions that require both permanent correction of a genomic lesion and ongoing modulation of a pathogenic transcript, of which Huntington's disease is a representative and motivating example.

### Description of the Related Art

### Single-Modality Genome and Transcriptome Editing has Advanced Toward the Clinic

Genome editing has progressed substantially beyond early double-strand-break nuclease approaches toward precision editors that install defined nucleotide changes without requiring double-strand breaks. Cytosine base editors, which deaminate cytosine to uracil within a programmable window and thereby effect a cytosine-to-thymine transition, were established as a class of programmable single-base editors [Komor 2016]. Adenine base editors, which convert adenine to inosine and thereby effect an adenine-to-guanine transition, extended the reach of base editing to a complementary set of transition mutations [Gaudelli 2017]. Subsequent protein engineering improved the catalytic kinetics and editing efficiency of these systems, as exemplified by the evolved adenine deaminase variant ABE8e [Richter 2020], and improved the precision, expression, and product purity of cytosine base editors through optimized constructs such as BE4max and AncBE4max [Koblan 2018]. These chemistries collectively enable correction of a large fraction of known pathogenic point mutations without generating the uncontrolled insertions and deletions associated with nuclease-mediated repair.

Prime editing further broadened the scope of precise genome modification by coupling a catalytically impaired Cas9 nickase to a reverse transcriptase and a prime-editing guide RNA, thereby enabling targeted insertions, deletions, and all twelve classes of point mutation without double-strand breaks or donor templates [Anzalone 2019]. Refinements to prime editing improved efficiency and reduced byproducts, including the PE5 and PEmax architectures and the transient inhibition of mismatch repair through an engineered dominant-negative MLH1 protein [Chen 2021]. Prime editing thus provides a versatile route to durable, programmable correction of genomic sequence in settings where base editing alone is insufficient.

In parallel, RNA-targeting effectors of the Cas13 family enable manipulation of the transcriptome without altering the genome. Cas13 enzymes provide programmable, RNA-guided knockdown of target transcripts [Abudayyeh 2017], and the compact Cas 13d ortholog, also referred to as CasRx, enables efficient and specific transcript reduction with a small effector suitable for delivery [Konermann 2018]. Catalytically inactivated Cas 13 has additionally been adapted for programmable adenosine-to-inosine RNA editing through the REPAIR system, permitting reversible, transcript-level correction [Cox 2017]. Cas 13-based approaches are accordingly well suited to conditions in which transient or tunable regulation of a transcript, rather than permanent genomic change, is desirable.

The clinical feasibility of single-modality CRISPR-based editing in human subjects is evidenced by multiple publicly reported clinical programs. Programs employing base editing of hematopoietic cells, in vivo base editing directed to lipid-regulating genes, in vivo nuclease-based editing of lipid-regulating genes, and additional in vivo editing programs directed to ophthalmic and other targets have entered human evaluation. These publicly reported clinical programs, including those identified as BEAM-101, VERVE-102, CTX310, PM359, HG202, and HG204, collectively demonstrate that base editing, nuclease editing, and related CRISPR modalities can be administered to human subjects. Each such program, however, deploys a single editing modality through a single delivery vehicle and does not address conditions requiring coordinated action of multiple distinct editing chemistries.

### DNA Nanotechnology Provides an Addressable, Compartmentalizable Chassis

Structural DNA nanotechnology provides a means to construct objects of defined size, shape, and surface chemistry from nucleic acids. The DNA origami method, in which a long single-stranded scaffold is folded into a target geometry by a set of short staple oligonucleotides, enables the fabrication of two-dimensional and three-dimensional shapes with nanometer-scale spatial addressability [Rothemund 2006]. The extension of this method to self-assembling three-dimensional shapes demonstrated that hollow and intricate volumetric architectures could be constructed reliably [Douglas 2009]. More recent work has demonstrated modular DNA origami nanocompartments capable of encapsulating molecular payloads within defined internal volumes [Huang 2024].

A salient feature of these DNA nanostructures is their capacity for internal compartmentalization and site-specific functionalization. Because each staple strand occupies a known position, payloads, recognition elements, and responsive sequence motifs can be placed at predetermined locations on or within the structure. This addressability permits the construction of internal chambers that physically separate distinct cargoes from one another, a property that is of particular value when the cargoes are enzymatically active species that should not interact prematurely.

### Logic-Gated and Cascaded DNA Nanodevices Permit Conditional Behavior

Beyond static carriers, DNA nanostructures have been engineered to undergo conditional conformational change in response to molecular inputs. A logic-gated DNA nanorobot was demonstrated to open and expose its payload only upon recognition of defined cell-surface inputs through an aptamer-based gating mechanism, thereby providing conditional, input-dependent release [Douglas 2012]. Cascaded and higher-order logic operations have likewise been implemented in DNA nanodevices, including a second-order logic-gated DNA nanorobot capable of integrating multiple sequential inputs to govern its actuation [Wang 2021]. These demonstrations establish that DNA nanostructures can serve not merely as passive containers but as responsive devices whose payload release is contingent upon the coincident or sequential satisfaction of defined molecular conditions.

### The Blood-Brain Barrier Is the Central Obstacle to Systemic CNS Therapeutics, and DNA Nanostructures Can Cross It

The blood-brain barrier presents the principal obstacle to the systemic delivery of therapeutics to the central nervous system. The barrier restricts the passage of macromolecules and nanoscale particles from the circulation into brain parenchyma, with the consequence that many otherwise promising agents fail to reach their intended targets when administered intravenously. Receptor-mediated transcytosis, in which a ligand engages a luminal receptor on brain endothelial cells and is transported across the cell, provides one of the few validated routes by which large cargoes may cross the barrier. The transferrin receptor is a well-characterized target for this purpose; engineered anti-transferrin-receptor binding moieties have been shown to mediate transcytosis across brain endothelium [Yu 2011], and transferrin-receptor-binding aptamers have been shown to promote penetration of the barrier [Li 2020].

Critically, DNA nanostructures themselves have been demonstrated to cross the blood-brain barrier. DNA nanocages have been shown to traverse the barrier and access brain tissue [Tam 2020], framework DNA nanostructures have been shown to cross an artificial blood-brain-barrier model [Chen 2022], and DNA tetrahedral nanocages have been used to deliver a neurologically relevant payload across the barrier [Singh 2024]. These results establish that a suitably engineered and surface-functionalized DNA nanostructure can serve as a vehicle for systemic delivery of cargo to the central nervous system, in contrast to invasive intraparenchymal or intrathecal routes.

### Unmet Need

Notwithstanding the independent validation of each of the foregoing technologies, no prior system unites them into a single coordinated therapeutic vehicle. Base editing, prime editing, and Cas13 RNA editing have each been advanced toward the clinic, but only as isolated single-modality interventions, each delivered through its own vehicle and acting independently. DNA origami provides an addressable and compartmentalizable chassis, and logic-gated DNA nanodevices provide conditional and cascaded actuation, yet these capabilities have not been harnessed to carry and coordinate multiple distinct genome-editing and transcriptome-editing chemistries. DNA nanostructures have been shown to cross the blood-brain barrier, yet such crossing has not been combined with multimodal editing cargo and conditional sequential release.

Accordingly, there remains an unmet need for a system that simultaneously provides, first, the co-delivery of multiple CRISPR modalities, namely a base editor, a prime editor, and a Cas13 RNA editor, within one vehicle; second, a compartmentalized internal architecture that physically separates these enzymatically active editors so as to prevent their premature interaction prior to release; third, systemic blood-brain-barrier crossing that enables noninvasive intravenous administration for delivery to the central nervous system; and fourth, logic-gated sequential release that temporally coordinates the action of the several modalities so that each acts in its appropriate order.

The clinical motivation for such a system is exemplified by Huntington's disease. Huntington's disease arises from an expanded trinucleotide repeat and is characterized by the production of a pathogenic protein whose ongoing expression drives progressive neurodegeneration. Addressing this disease optimally calls for both a permanent correction of the underlying genomic lesion and an ongoing regulation of the pathogenic transcript so that residual or newly expressed pathogenic species are continuously suppressed. No single editing modality satisfies both requirements at once: permanent correction is the province of base or prime editing, whereas tunable, ongoing transcript suppression is the province of Cas13. The recognition that Huntington's disease and analogous conditions implicate both the genome and the transcriptome has been reflected in efforts directed at the trinucleotide repeat and its transcript, including Cas13d approaches to Huntington's disease phenotypes [Morelli 2023], CasRx-based approaches to Huntington's disease [Lin 2025], and base-editing strategies that alter the repeat sequence such as conversion of the pathogenic codon configuration [Choi 2024] and base editing of trinucleotide repeats more generally [Matuszek 2025].

The history of single-modality and invasive approaches to such conditions further teaches the insufficiency of the prior art. Antisense oligonucleotide programs directed at the pathogenic transcript, despite achieving transcript knockdown, have not delivered the durable disease modification sought, illustrating the limits of a transient single-modality strategy. Surgically delivered, virally vectored approaches requiring direct intraparenchymal administration impose invasive delivery and constrained distribution, illustrating the limits of an invasive single-vehicle strategy. Together these experiences teach that neither a single editing modality nor an invasive single-vehicle delivery is, by itself, sufficient to meet the clinical need. There thus remains a need for an integrated, compartmentalized, systemically administrable, and temporally coordinated multimodal editing system, which the present invention provides.

### SUMMARY OF THE INVENTION

### Brief Summary of the Invention

The present invention provides a compartmentalized DNA origami nanorobot that integrates multiple discrete CRISPR-based editing modalities within a single systemically administrable vehicle, together with associated methods of treatment and methods of manufacture. The system is configured to traverse the blood-brain barrier following intravenous administration, to enter target cells of the central nervous system, and to release its several editing cargoes in a defined and conditionally gated sequence, thereby coordinating permanent genomic correction with ongoing transcript regulation in a manner not achievable by any single modality acting alone.

In one general aspect, the invention provides a substantially spherical DNA nanocage having an external dimension on the order of about 100 to about 140 nanometers and defining at least three discrete internal compartments. A first compartment is configured to contain a base editor; a second compartment is configured to contain a prime editor; and a third compartment is configured to contain a Cas13 RNA editor. The compartments are physically segregated within the nanocage such that the enzymatically active editing complexes are isolated from one another and are thereby configured to prevent premature interaction among the modalities prior to their intended release. The aggregate editing-complex cargo carried within the nanocage is configured to exceed approximately 500 kilodaltons, reflecting the simultaneous accommodation of three distinct multi-component editing systems within a single structure.

In a further aspect, the exterior surface of the nanocage is functionalized to enable systemic administration and crossing of the blood-brain barrier. The surface is configured to present a plurality of transferrin-receptor-binding aptamers, in certain configurations numbering between about eighteen and about thirty, which are configured to engage the transferrin receptor on brain endothelial cells and thereby mediate receptor-mediated transcytosis across the barrier. The surface is further configured to bear a polyethylene glycol coating, which is configured to reduce nonspecific interactions, improve circulation behavior, and support intravenous administration. The combination of transferrin-receptor aptamer presentation and PEGylation is thus configured to permit noninvasive delivery of the multimodal cargo to the central nervous system.

In a further aspect, the invention provides a three-tier sequential logic-gated release mechanism in which the conditional opening of the nanocage and the release of its compartmentalized cargoes are governed by the coincident detection of multiple distinct molecular inputs at successive gates. Each gate is configured to require at least two distinct molecular inputs, such that release proceeds only upon satisfaction of an effective logical conjunction at each stage. In representative configurations, a first gate is configured to require detection of a disease-associated biomarker together with detection of a cell-type-specific marker; a second gate is configured to require confirmation of first-gate satisfaction together with detection of an intracellular pH below approximately 6.5, the latter being sensed through a pH-responsive i-motif DNA element [Gehring 1993]; and a third gate is configured to require confirmation of second-gate satisfaction together with recognition associated with nuclear localization, which in certain configurations is mediated by a nuclear-localization signal such as the SV40 nuclear-localization signal [Kalderon 1984]. This multi-input gating architecture builds upon the conditional and cascaded actuation demonstrated for logic-gated DNA nanodevices [Douglas 2012][Wang 2021] and is configured to confer specificity by requiring multiple independent conditions to be met before cargo is exposed.

In a further aspect, the gating architecture is configured to effect temporal coordination of the several editing modalities. The release mechanism is configured such that the base editor is released first, the prime editor is released second, and the Cas13 RNA editor is released third, whereby permanent genomic editing operations are configured to precede, and the ongoing transcript-regulating operation is configured to follow, in an ordered cascade. This temporal sequencing is configured to allow the durable genomic correction to be established before the transcript-level modality assumes an ongoing regulatory role.

In a further aspect, the coordinated multimodal action of the system is configured to produce a multiplicative therapeutic effect that exceeds what any single modality is expected to achieve. By combining permanent correction of a genomic lesion through base and prime editing with ongoing suppression or correction of the pathogenic transcript through Cas13, the system is configured to achieve, in representative configurations and as an expected outcome, a reduction of greater than approximately ninety percent in the burden of a pathogenic protein in conditions such as Huntington's disease. Such an outcome is configured to arise from the synergy of the combined modalities and is not expected to be attainable by any single modality administered alone. The invention does not depend upon, and the foregoing statements are not to be construed as, any absolute guarantee of efficacy; rather, the system is configured and expected to provide the described coordinated multimodal effect.

The invention is set forth in three independent claim categories. A first category is directed to the system or composition of matter, namely the compartmentalized DNA origami nanorobot comprising the spherical DNA nanocage, the three discrete editing compartments and their respective base editor, prime editor, and Cas13 RNA editor cargoes, the surface transferrin-receptor aptamers and PEGylation, and the three-tier logic-gated release architecture. A second category is directed to a method of treatment, comprising administering the system to a subject and effecting blood-brain-barrier crossing, cellular entry, conditional multi-input gated release, and temporally coordinated multimodal editing in target cells of the central nervous system. A third category is directed to a method of manufacture, comprising assembling the DNA nanocage, defining and loading the discrete compartments with their respective editing complexes, and functionalizing the surface with the transferrin-receptor aptamers and polyethylene glycol coating. These categories, and the configurations described herein, are detailed further in the description that follows.

### DETAILED DESCRIPTION OF THE INVENTION

### Enabling Disclosure and Scope

The present invention is enabled in its entirety by the integration of validated, commercially available technologies, each of which is well established in the art and routinely practiced by persons of ordinary skill. The structural chassis of the platform is constructed using standard DNA origami self-assembly, a methodology that has been reduced to reliable laboratory practice and that proceeds through the predictable folding of a long single-stranded scaffold by a stoichiometric excess of short complementary staple oligonucleotides [Rothemund 2006][Douglas 2009][Huang 2024]. The CRISPR editing complexes housed within the platform are produced by established recombinant protein expression in bacterial, insect, or mammalian systems and by established solid-phase or in vitro transcription synthesis of guide RNA species, both of which are mature, scalable, and commercially serviced. No step disclosed herein requires an extraordinary, speculative, or unpredictable operation; every functional building block has been independently demonstrated and is assembled here in a defined and reproducible manner to yield the integrated system.

Characterization and quality control of the assembled nanostructure are likewise accomplished with conventional instrumentation. Transmission electron microscopy and atomic force microscopy resolve the size, shape, and compartmental organization of individual particles, while dynamic light scattering reports the hydrodynamic diameter and polydispersity of the particle population in suspension. Agarose gel electrophoresis confirms scaffold-staple incorporation and folding yield. These methods are standard nanoparticle characterization techniques whose outputs are interpreted according to well-known principles, and a person of ordinary skill in the art can therefore verify that a given preparation conforms to the dimensions and architecture set forth in this specification without undue experimentation.

The biological activity of the loaded editing complexes is confirmed using established cell-based editing assays. Editing outcomes at targeted genomic loci are quantified by next-generation sequencing of amplicons spanning the edit site, permitting precise determination of base conversion frequency, prime-edited allele frequency, and the absence or presence of unintended insertions or deletions. Transcript-level effects, including knockdown mediated by the RNA-targeting complex, are quantified by reverse transcription quantitative polymerase chain reaction. Together these readouts provide a complete and quantitative account of platform performance at the molecular level. Because each constituent technology is validated and commercially available, and because their combination follows directly from the teachings herein, the full scope of the claimed invention is enabled.

Certain conventions of construction apply throughout this specification. The section and subsection headings used herein are provided solely for convenience of organization and do not limit the scope or interpretation of any disclosure or claim. The term “comprising,” and its grammatical variants, is used in its open-ended sense and does not exclude additional unrecited elements or steps. Every numerical range disclosed herein is intended to encompass and to provide written description support for each and every subrange and individual value falling within the stated endpoints, whether or not such subrange or value is separately recited. Finally, the various embodiments, features, compartments, ligands, control elements, and method steps described herein may be combined with one another in any operable permutation, and the description of a feature in the context of one embodiment shall be understood to support its incorporation into any other embodiment unless such combination is technically incompatible.

### Definitions

### Defined Terms

As used herein, the terms “nanorobot” and “nanostructure” refer interchangeably to an engineered particle of nanometer-scale dimensions that possesses a defined three-dimensional architecture and that is configured to carry out one or more programmed functions, including molecular recognition, conditional opening of internal compartments, and controlled release of an encapsulated payload. In the context of the present invention, the nanorobot is a self-assembled nucleic acid assembly bearing surface targeting ligands and an internal logic-gated control system, such that it senses defined environmental inputs and responds with a deterministic mechanical and biochemical output.

As used herein, the term “DNA origami” refers to the technique, and to the resulting structures, in which a long single-stranded nucleic acid scaffold is folded into a predetermined two-dimensional or three-dimensional shape through hybridization with a plurality of shorter complementary oligonucleotides, the spatial arrangement of folds being dictated by the designed sequence complementarity between the scaffold and the shorter strands [Rothemund 2006][Douglas 2009]

As used herein, the term “scaffold strand” refers to the long single-stranded nucleic acid that constitutes the continuous backbone of a DNA origami structure and that is routed back and forth through the designed object to define its overall geometry. In preferred embodiments the scaffold strand is the M13mp18 bacteriophage genome of 7,249 nucleotides, a well-characterized scaffold widely employed in the art.

As used herein, the term “staple strand” refers to any of the plurality of short synthetic oligonucleotides that bind to spatially separated regions of the scaffold strand and thereby crosslink and fix the scaffold into its intended folded conformation. Individual staple strands may be chemically modified or extended to display functional groups, capture sequences, or attachment handles at defined positions on the assembled structure.

As used herein, the term “compartment” refers to a discrete, walled internal cavity defined within the assembled nanostructure, the walls of the compartment being formed from bundled DNA double helices and the cavity being dimensioned to enclose a designated molecular payload. Each compartment is independently addressable and is sealed by a movable lid whose opening is governed by the control system of the platform.

As used herein, the term “base editor” refers to a programmable enzyme complex capable of installing a targeted single-base substitution in genomic DNA without generating a double-strand break, comprising a catalytically impaired CRISPR-associated targeting protein fused to a nucleobase deaminase. An adenine base editor, or ABE, mediates the conversion of an adenine-thymine base pair to a guanine-cytosine base pair through deamination of adenine to inosine [Gaudelli 2017]. A cytosine base editor, or CBE, mediates the conversion of a cytosine-guanine base pair to a thymine-adenine base pair through deamination of cytosine to uracil [Komor 2016]. The activity, scope, and therapeutic relevance of such base editors are well established in the art [Richter 2020][Koblan 2018].

As used herein, the term “prime editor” refers to a programmable editing complex comprising a CRISPR-associated nickase fused to an engineered reverse transcriptase, which complex installs targeted insertions, deletions, and all classes of base-to-base conversions without requiring a donor template or a double-strand break [Anzalone 2019][Chen 2021]. As used herein, the term “pegRNA,” or prime editing guide RNA, refers to the extended guide RNA employed by a prime editor that both specifies the genomic target and templates the desired edit through a primer binding site and a reverse transcription template appended to its three-prime end.

As used herein, the term “Cas13 RNA editor” refers to a class of RNA-guided, RNA-targeting CRISPR effector proteins that bind and act upon single-stranded RNA, and that may be configured for transcript cleavage and knockdown or for programmable nucleotide-level RNA editing [Abudayyeh 2017][Konermann 2018][Cox 2017]. As used herein, the term “crRNA,” or CRISPR RNA, refers to the guide RNA that directs a Cas13 RNA editor to its complementary target transcript through Watson-Crick base pairing.

As used herein, the term “logic gate” refers to a molecular control element that receives one or more defined molecular inputs and produces a defined output, such as the opening of a compartment lid, only when the inputs satisfy a specified logical condition. As used herein, an “AND gate” is a logic gate that produces its output only when all of its required inputs are simultaneously present, such that conditional and sequential actuation of the nanostructure is achieved [Douglas 2012].

As used herein, the term “toehold-mediated strand displacement” refers to a dynamic nucleic acid process in which an invading single strand binds to an exposed single-stranded toehold region of a partially double-stranded complex and thereafter progressively displaces an incumbent strand through branch migration, thereby effecting a programmed conformational change such as the unlatching of a compartment lid.

As used herein, the term “i-motif” refers to a four-stranded, intercalated secondary structure that forms within cytosine-rich nucleic acid sequences under mildly acidic conditions through hemiprotonated cytosine-cytosine base pairing, and that reversibly unfolds upon return to neutral or basic conditions, thereby serving as a pH-responsive structural switch within the platform [Gehring 1993].

As used herein, the term “nuclear localization signal,” or NLS, refers to a short peptide sequence that directs the active transport of an attached cargo from the cytoplasm into the cell nucleus through the nuclear pore complex, thereby promoting delivery of the genome-editing complexes to their site of action [Kalderon 1984].

As used herein, the term “transferrin receptor aptamer,” or TfR aptamer, refers to a nucleic acid ligand selected to bind the transferrin receptor with high affinity and specificity, the binding of which triggers receptor-mediated uptake of the displaying nanostructure and supports its passage across cellular barriers that express the receptor.

As used herein, the term “transcytosis” refers to the vesicular transport process by which a macromolecular cargo is taken up at one face of a polarized cell, conveyed across the cell interior within membrane-bound vesicles, and released at the opposite face, thereby traversing a cellular barrier without disrupting its integrity.

As used herein, the term “blood-brain barrier,” or BBB, refers to the selectively permeable interface formed by specialized brain microvascular endothelial cells, together with associated pericytes and astrocytic end-feet, that tightly regulates the passage of substances between the systemic circulation and the central nervous system parenchyma and that ordinarily excludes large therapeutic macromolecules.

As used herein, the term “PEGylation” refers to the covalent attachment of one or more polyethylene glycol moieties to the surface of the nanostructure, which attachment confers a hydrophilic stealth layer that reduces nonspecific protein adsorption, mitigates recognition by the mononuclear phagocyte system, and prolongs systemic circulation.

### System Overview

The platform of the present invention is, in narrative summary, a self-assembled spherical DNA origami nanocage having an outer diameter in the range of 100 to 140 nanometers, with broader operable embodiments spanning a claim range of 90 to 200 nanometers. The nanocage is folded from a single-stranded M13mp18 scaffold of 7,249 nucleotides together with a complementary set of staple strands, and its interior is partitioned into three discrete internal compartments. The first compartment, designated C1, has approximate external dimensions of 30 by 30 by 40 nanometers, enclosing a volume of approximately 36,000 cubic nanometers, and is sized to house a base editor complex of approximately 180 to 200 kilodaltons. The second compartment, designated C2, has approximate external dimensions of 30 by 30 by 45 nanometers, enclosing a volume of approximately 40,500 cubic nanometers, and is sized to house a prime editor complex of approximately 220 to 250 kilodaltons. The third compartment, designated C3, has approximate external dimensions of 28 by 28 by 35 nanometers, enclosing a volume of approximately 27,440 cubic nanometers, and is sized to house a Cas13 RNA editor complex of approximately 130 to 150 kilodaltons. In aggregate the encapsulated cargo exceeds 500 kilodaltons, a payload mass far beyond the reach of conventional delivery vehicles.

Each compartment is bounded by walls formed from bundled DNA double helices, which provide the rigidity required to maintain a defined internal cavity and to protect the enclosed editing complex from premature degradation. The mouth of each compartment is closed by an independently addressable lid, such that the three compartments can be opened separately and in a programmed order rather than discharging their contents simultaneously. The three compartments are held in spatial relationship to one another within the cage by flexible poly-thymidine linkers, which accommodate the conformational rearrangements attending lid actuation while preserving the overall integrity of the assembly. This compartmentalized architecture is the structural basis for the temporally staged, multi-modality editing function of the platform.

The outer surface of the nanocage is functionalized to serve two complementary purposes, namely targeting and stealth. For targeting, the surface displays a plurality of transferrin receptor aptamers, in preferred embodiments numbering from 18 to 30 and in broader embodiments numbering from 12 to 40, which aptamers mediate recognition of transferrin-receptor-bearing endothelium and cells. For stealth and circulation persistence, the surface bears a coating of polyethylene glycol together with cholesterol anchoring moieties, which coating reduces opsonization and immune recognition and thereby extends the systemic residence time of the particle. The balance between dense targeting ligand presentation and an effective stealth layer is selected to favor barrier crossing while limiting nonspecific clearance.

Coordinating the behavior of the three compartments is a three-tier logic-gated control system that governs the sequence in which the compartment lids open. Rather than releasing all payloads at once, the control system imposes an ordered cascade by requiring distinct molecular conditions to be satisfied at successive tiers, with each tier acting as a gate that authorizes the opening of a particular compartment. The control elements are implemented through nucleic acid switches and strand-displacement mechanisms responsive to defined intracellular cues, such that the platform performs as a conditional, programmable device rather than a passive carrier.

The temporal operation of the platform may be summarized as a defined cascade. Following intravenous administration, the nanocage circulates within the bloodstream protected by its stealth coating. Upon encountering the brain microvascular endothelium, the surface transferrin receptor aptamers engage their cognate receptor and trigger transferrin-receptor-mediated transcytosis, by which the intact particle is conveyed across the blood-brain barrier into the central nervous system parenchyma. Once within the target tissue, the particle undergoes cellular internalization, after which the three-tier control system actuates the compartment lids in a programmed order. The base editor compartment opens first to deliver its complex for precise single-base correction, the prime editor compartment opens next to deliver its complex for templated edits, and the Cas13 RNA editor compartment opens last to deliver its complex for transcript-level modulation. This staged release allows complementary editing modalities to act in a designed sequence rather than in uncontrolled coincidence.

The platform is modular and reconfigurable. Because the targeting ligands, the compartment payloads, and the guide sequences are each independently specifiable, the same structural chassis and control architecture can be adapted to a wide variety of genetic neurological diseases simply by exchanging the editing complexes and their guides for those appropriate to a given target. Huntington's disease is described herein as a lead embodiment that illustrates the coordinated action of base editing, prime editing, and RNA-level modulation against a defined neurological target, while it is understood that the same platform extends to many additional inherited and acquired disorders of the central nervous system.

### Scaffold Design

The structural foundation of the disclosed nanorobot is a programmable deoxyribonucleic acid origami architecture in which a single long strand of single-stranded DNA, termed the scaffold, is folded into a defined three-dimensional shape through hybridization with a population of shorter complementary oligonucleotides. In the preferred embodiment, the scaffold is the genome of the M13mp 18 bacteriophage, a circular single-stranded DNA molecule of 7,249 nucleotides. This scaffold is selected because its sequence is fully characterized, its length is well matched to the volumetric requirements of the present multi-compartment cage, and its circular topology removes free ends that would otherwise be vulnerable to exonucleolytic attack and that would complicate routing of the folded path. The use of a defined-sequence biological scaffold of this kind, rather than an arbitrary or randomly generated sequence, s what permits the deterministic, sequence-addressable construction of complex two-dimensional and three-dimensional objects from DNA [Rothemund 2006].

The choice of the M13mp18 genome confers several practical advantages relevant to a therapeutic platform. The scaffold is available at pharmaceutical scale from established commercial suppliers and from bacteriophage propagation, providing a reproducible and economically tractable starting material whose batch-to-batch sequence identity is invariant. Single-stranded DNA derived from the M13 system has been employed extensively as the scaffold for folded DNA objects intended for biological environments, and the resulting structures exhibit low intrinsic immunogenicity relative to many proteinaceous or viral delivery vehicles, an attribute of substantial importance for a construct intended to cross the blood-brain barrier and persist transiently within the central nervous system [Douglas 2009]. Because the scaffold is composed entirely of natural nucleotides, it is ultimately biodegradable through endogenous nuclease activity once its therapeutic function is complete, which limits long-term accumulation.

While the 7,249-nucleotide M13mp18 scaffold is preferred for the assembled cage described herein, the architecture is not limited to a single scaffold length. For embodiments requiring a larger internal volume, additional compartments, or a thicker multi-helix wall, longer scaffolds may be substituted, including the p7308 derivative of approximately 7,308 nucleotides, the p8064 derivative of approximately 8,064 nucleotides, and custom enzymatically or synthetically produced single-stranded DNA scaffolds ranging from approximately two kilobases to ten kilobases. Shorter custom scaffolds may be used where a smaller or simpler compartment is sufficient. The selection of scaffold length is governed by the total contour length of DNA required to enclose the intended cargo volume at the chosen wall thickness, such that a scaffold is chosen whose length, after folding, yields a closed cage of the target outer dimension.

Routing of the scaffold path and assignment of the complementary folding oligonucleotides are performed using established computer-aided design environments for structural DNA nanotechnology. In the preferred workflow, the lattice-based design environment caDNAno is employed to lay out the helical bundles of each compartment wall, to define crossover positions between adjacent helices, and to generate the corresponding set of folding oligonucleotide sequences. For curved, spherical, and free-form surfaces such as the closed outer cage of the present construct, a surface-meshing design environment such as vHelix is additionally employed to route the scaffold over an arbitrary triangulated surface and to relax the resulting structure into a physically realizable wireframe. These tools translate a desired geometry into an explicit list of oligonucleotide sequences, each of which addresses a unique set of positions along the scaffold.

A further advantage of the scaffolded origami approach adopted here is that mature, well-designed DNA origami objects assemble in a single annealing operation at high yield, with the great majority of scaffold strands incorporating into the intended structure rather than into kinetically trapped or aggregated byproducts. This high routine yield is a direct consequence of the cooperative, sequence-specific nature of scaffold folding, and it is what makes the multi-compartment construct of the present disclosure manufacturable at the quantities required for a therapeutic agent. The detailed thermal and purification procedures by which assembly is accomplished are addressed elsewhere in this specification; the present section is concerned with the architecture of the folded object rather than with the process of folding it.

### Staple Strand Architecture

The scaffold alone does not define a shape. The three-dimensional form of the nanorobot emerges only when the scaffold is hybridized to a designed set of short complementary oligonucleotides, referred to throughout this disclosure as staple strands. In the preferred embodiment, the complete construct is folded by a population of approximately 200 to 300 distinct staple strands, each typically between 20 and 60 nucleotides in length. Each staple strand carries a sequence that is complementary to several non-contiguous segments of the scaffold, so that when the staple anneals it draws those distant scaffold segments into spatial proximity and locks them against one another.

The staple strands employed in a therapeutic embodiment are produced by pharmaceutical-grade solid-phase oligonucleotide synthesis under controlled conditions, and they are purified to remove truncated failure sequences and synthesis byproducts. Depending on the length and the presence of chemical modifications, purification is accomplished by high-performance liquid chromatography or by denaturing polyacrylamide gel electrophoresis, each staple being held to a defined purity specification so that the folding population is compositionally reproducible from batch to batch. The reproducibility of this oligonucleotide set, taken together with the invariant scaffold sequence, is what allows the finished nanorobot to be manufactured as a defined molecular entity.

The mechanism by which the staples impose a shape is the antiparallel crossover. Each staple, in the course of binding its several complementary scaffold segments, passes from one DNA double helix into an adjacent double helix at a defined position, thereby tying the two helices together at that point. By placing many such crossovers at regular intervals throughout the structure, the otherwise independent helices are constrained into a rigid bundle, and the bundle in turn is constrained into the intended surface or wall. The crossovers therefore serve simultaneously as the folding instructions and as the mechanical fasteners of the assembled object.

Crossover placement is not arbitrary. The geometry of the DNA double helix dictates that a crossover can be formed without introducing torsional strain only where the backbones of two neighboring helices face one another, and this condition recurs at a fixed angular and axial periodicity along the helix. In the present construct, crossovers are accordingly spaced at intervals of approximately 7 to 21 base pairs along each helix, the precise spacing being chosen to match the lattice geometry of the particular compartment wall. The use of such patterned crossovers to weave a scaffold into a programmed shape is the foundational principle of scaffolded DNA origami [Rothemund 2006], and its extension into densely packed three-dimensional helix bundles is what permits the rigid, volumetric compartments described below [Douglas 2009].

Two principal lattice geometries are contemplated for the compartment walls. In a honeycomb lattice, each helix is surrounded by three nearest neighbors and crossovers recur with a periodicity consistent with a 21-base-pair structural repeat, yielding walls of comparatively low bending stiffness and lower DNA mass per unit volume. In a square lattice, each interior helix is surrounded by four nearest neighbors and crossovers are placed on a denser, approximately 21-base-pair adjusted spacing appropriate to the orthogonal packing, yielding stiffer, more densely packed walls suited to compartments that must resist deformation while protecting an enzymatic cargo. The honeycomb lattice is preferred for the curved outer cage and for flexible interconnections, whereas the square lattice is preferred for the load-bearing internal compartment walls; the two may be combined within a single construct.

A defined subset of the staple population is functionalized rather than structural. These functional staples carry, at their 3′ or 5′ termini, single-stranded extensions referred to as handles that project away from the folded surface and that are not consumed in scaffold hybridization. Handles directed toward the interior of a compartment serve as capture sequences that hybridize to complementary docking sequences appended to the editing-complex cargo, thereby holding each cargo at an addressable interior position. Handles directed toward the exterior surface serve other functions: certain functional staples are extended to present nucleic acid aptamer sequences used in the cargo-retaining lid mechanisms and in surface recognition, while others are conjugated to cholesterol-modified polyethylene glycol moieties that confer membrane affinity and colloidal stabilization. Because each functional staple occupies a unique, predetermined position in the design, the spatial arrangement of capture sites, aptamers, and surface ligands is fully programmable.

### Three-Compartment Architecture

The assembled nanorobot is a closed, approximately spherical DNA origami cage measuring between 100 and 140 nanometers in outer diameter. Within this outer cage are constructed three discrete internal compartments, each a hollow box-like enclosure bounded by walls of bundled DNA double helices, and each dedicated to housing one of the three distinct gene-editing complexes carried by the platform. The three compartments are arranged within the cage interior and joined to one another and to the cage shell by flexible linkers, so that the construct presents externally as a single unified particle while internally maintaining three physically isolated reaction chambers.

The central architectural purpose of this three-compartment design is spatial segregation of the editing machinery. The platform carries a cytosine or adenine base-editor complex, a prime-editor complex, and a Cas13 RNA-editor complex simultaneously, and these complexes share overlapping affinities for nucleic acids and would, if co-confined in a single chamber, compete for guide RNAs, interfere with one another, and risk premature or off-target activity before the construct reaches its intended site of action. By enclosing each complex within its own walled compartment and gating each compartment with an independent lid responsive to a distinct trigger, the architecture keeps the three editors physically separated until each is individually licensed to release its payload. The viability of constructing modular DNA origami nanocompartments that spatially confine and independently co-localize functional macromolecular machinery, while preventing cross-interference between the confined species, has been established for origami-based compartment systems and underlies the present three-chamber design [Huang 2024].

The three compartments differ deliberately in their internal dimensions, in the editing complex each holds, and in the lid mechanism that gates each. These differences are detailed in the following subsections. In every case the compartment wall is a bundle of between two and eight parallel DNA double helices, the wall helix count being selected so that the wall is thick enough to occlude its enclosed cargo from the surrounding environment and rigid enough to maintain compartment shape, while not so massive as to inflate the overall particle beyond the target outer diameter.

### Compartment 1—Base Editor Module

The first compartment houses the base-editor complex, designated C1. The internal cavity of this compartment measures approximately 30 by 30 by 40 nanometers, providing an enclosed internal volume of approximately 36,000 cubic nanometers. This volume is dimensioned to accommodate the base-editor complex, which occupies a footprint of approximately 30 by 30 by 40 nanometers and has a molecular mass on the order of 180 to 200 kilodaltons, comprising a catalytically impaired CRISPR-associated nuclease fused to a nucleotide deaminase domain together with its bound guide RNA. The compartment provides cargo capacity sufficient to retain this complex of approximately 180 to 200 kilodaltons with margin for the interior capture oligonucleotides that dock it in place.

The walls of the first compartment are constructed from bundles of between two and eight parallel DNA double helices in a predominantly square-lattice arrangement, chosen to confer the rigidity required to protect the deaminase activity of the enclosed editor from premature exposure. The compartment is gated by a toehold-mediated dual-aptamer logic lid, in which two independent nucleic acid aptamer recognition events must both occur before a toehold-initiated strand-displacement cascade unzips the lid and opens the compartment. This dual-aptamer AND configuration requires the simultaneous presence of two molecular inputs to license opening, providing a stringent two-input control over base-editor release. Within the assembled cage, the first compartment occupies one of the three interior positions and is tethered to its neighboring compartments and to the cage shell by flexible linkers, holding the base-editor module isolated from the prime-editor and Cas13 modules until its dual-aptamer condition is satisfied.

### Compartment 2—Prime Editor Module

The second compartment houses the prime-editor complex, designated C2. Its internal cavity measures approximately 30 by 30 by 45 nanometers, yielding an enclosed internal volume of approximately 40,500 cubic nanometers, the largest of the three compartments. This greater volume reflects the larger size of the prime-editing machinery, which occupies a footprint of approximately 30 by 30 by 45 nanometers and has a molecular mass on the order of 220 to 250 kilodaltons, comprising a nickase-form CRISPR-associated nuclease fused to an engineered reverse transcriptase domain together with its associated prime-editing guide RNA. The compartment provides cargo capacity sufficient to retain this complex of approximately 220 to 250 kilodaltons together with its interior docking oligonucleotides.

The walls of the second compartment are likewise constructed from bundles of between two and eight parallel DNA double helices, with helix count and lattice selected to enclose and protect the larger prime-editing complex while maintaining compartment shape. The second compartment is gated by an i-motif pH-sensitive lid. The i-motif is a four-stranded cytosine-rich DNA secondary structure that folds and unfolds reversibly as a function of local pH, adopting its folded, intercalated conformation under mildly acidic conditions and relaxing under neutral-to-basic conditions; this characteristic pH-dependent conformational transition of cytosine-rich sequences provides the conformational switch that opens or closes the lid in response to the local proton concentration [Gehring 1993]. The pH transition thereby couples prime-editor release to the chemical environment encountered along the intracellular trafficking pathway. Within the assembled cage, the second compartment occupies a second interior position adjacent to the first and third compartments, connected to them by flexible linkers, so that its acid-triggered opening proceeds independently of the triggers governing the other two modules.

### Compartment 3—Cas13 RNA Editor Module

The third compartment houses the Cas13 RNA-editor complex, designated C3. Its internal cavity measures approximately 28 by 28 by 35 nanometers, providing an enclosed internal volume of approximately 27,440 cubic nanometers, the smallest of the three compartments. This volume is matched to the Cas13 complex, which occupies a footprint of approximately 28 by 28 by 35 nanometers and has a molecular mass on the order of 130 to 150 kilodaltons, comprising a programmable RNA-targeting CRISPR-associated effector together with its bound CRISPR RNA. The compartment provides cargo capacity sufficient to retain this complex of approximately 130 to 150 kilodaltons and its interior capture oligonucleotides.

The walls of the third compartment are again constructed from bundles of between two and eight parallel DNA double helices selected to enclose and protect the RNA-targeting effector. The third compartment is gated by a nuclear-localization-signal and importin-triggered lid. In this mechanism, the lid bears or sequesters a nuclear-localization-signal peptide, and recognition of that signal by the cellular importin nuclear-transport machinery serves as the trigger that licenses opening of the compartment; the defined short basic peptide sequences that function as transferable nuclear-localization signals recognized by the importin import apparatus provide the molecular address that gates this lid [Kalderon 1984]. Coupling the third compartment to importin recognition links Cas13 release to engagement with the nuclear-transport pathway. Within the assembled cage, the third compartment occupies the remaining interior position, tethered by flexible linkers to the first and second compartments and to the cage shell, completing the three-chamber arrangement and maintaining the Cas13 module in isolation until its importin-dependent condition is met.

### Structural Design Principles

The compartment walls throughout the construct are formed of bundled double helices rather than single duplexes. Bundling multiple parallel double helices and stitching them with periodic crossovers produces a wall whose flexural rigidity greatly exceeds that of an isolated DNA duplex, so that each compartment behaves as a stiff box rather than a floppy bag. The number of helices in a given wall, between two and eight in the present construct, is chosen as a deliberate trade between rigidity and mass: thicker walls better occlude and protect their enclosed editing complex but consume more scaffold and staple material and enlarge the overall particle, and the wall counts specified for the three compartments represent the balance struck for each module's protective requirement.

The interior of each compartment is furnished with capture oligonucleotides, which are the interior-directed handles described above projecting inward from functional staples at predetermined positions. Each editing complex is appended with a docking oligonucleotide whose sequence is complementary to the capture oligonucleotide of its intended compartment, so that the complex hybridizes only at its designated interior site. Because every interior handle occupies a unique, designed location, cargo docking is fully addressable: a given editing complex binds at a known position within a known compartment, and no complex can mistakenly occupy a compartment intended for another. This addressability is the structural basis for loading the three editors independently of one another.

The three compartments are joined to one another, and the compartment assembly is joined to the outer cage shell, by flexible single-stranded linkers composed of poly-thymidine sequence approximately 15 to 30 nucleotides in length. Single-stranded poly-thymidine is chosen for these linkers because it is conformationally compliant and largely free of secondary structure, allowing the compartments controlled relative motion while holding them in defined topological relationship within the cage. These flexible tethers permit the compartments to flex and reorient with respect to one another and to the shell without rigidly fixing them, accommodating the conformational changes attendant on lid opening and cargo release while preventing the compartments from dissociating from the assembly.

The fully assembled construct is an approximately spherical object of 100 to 140 nanometers in outer diameter. This size and shape are not incidental but are deliberately selected to fall within the validated dimensional and morphological windows for crossing the blood-brain barrier, within which particle diameter and shape govern the efficiency of transcytosis across the cerebral endothelium [Tam 2020][Chen 2022]. By constraining the outer geometry to this window, the architecture positions the nanorobot to engage the receptor-mediated transcytosis pathways exploited for central-nervous-system delivery while remaining large enough to enclose its three-compartment cargo.

Several structural strategies confer the mechanical and chemical stability required of an in vivo agent. The folded object is held together cooperatively by the entire staple population, so that local thermal fluctuations cannot unzip the structure as they might a single short duplex, and the densely crossovered helix bundles resist mechanical deformation. The folded state is magnesium-dependent: divalent magnesium cations screen the electrostatic repulsion between the closely packed, negatively charged DNA backbones and are required to maintain the compact folded conformation, so the construct is formulated and stored under conditions that preserve adequate divalent cation occupancy. To resist nucleolytic degradation in biological fluids, the construct may be further protected by the cholesterol-polyethylene glycol surface coating presented by functional staples, by chemical modification or ligation of exposed staple termini, and by the inherent compactness of the densely packed bundles, which sterically occludes nuclease access to the interior helices. Together these strategies preserve structural integrity through circulation, transcytosis, and intracellular trafficking until the gated compartments are licensed to open.

A final and defining structural property is independent addressability of each compartment. Because the scaffold position of every functional staple is known, the capture sites of each compartment are sequence-distinct, the surface ligands are placed at designed positions, and each lid responds to its own dedicated trigger, every compartment can be loaded independently of the others during manufacture and gated independently of the others during operation. The base-editor, prime-editor, and Cas13 modules are therefore not merely co-packaged but separately programmable, each opening only when its specific molecular condition is satisfied, which is the structural foundation of the platform's controlled, sequential, multi-editor function.

### Cargo-Capacity Budgeting

The internal volumes of the three compartments are dimensioned in concert to accommodate the combined mass and bulk of all three editing complexes within a single particle. The base-editor complex of approximately 180 to 200 kilodaltons resides in the approximately 36,000-cubic-nanometer first compartment, the prime-editor complex of approximately 220 to 250 kilodaltons resides in the approximately 40,500-cubic-nanometer second compartment, and the Cas13 complex of approximately 130 to 150 kilodaltons resides in the approximately 27,440-cubic-nanometer third compartment. Summing the three editing complexes yields a total carried cargo mass in excess of 500 kilodaltons, distributed across roughly 103,940 cubic nanometers of combined internal compartment volume.

This budgeting confirms that each compartment is individually large enough to enclose its assigned complex with margin for the interior capture oligonucleotides and for the conformational freedom required during lid actuation, and that the three compartments together, nested within the 100 to 140 nanometer outer cage, accommodate a total cargo of greater than 500 kilodaltons. The progressive sizing of the compartments, largest for the prime editor, intermediate for the base editor, and smallest for the Cas13 effector, tracks the relative masses and footprints of the three complexes, so that internal volume is allocated where it is needed without inflating the assembled particle beyond the validated blood-brain-barrier-crossing size window.

### ASSEMBLY, CARGO LOADING, AND SURFACE FUNCTIONALIZATION

The present section provides a complete and enabling description of the methods by which the DNA origami nanorobot disclosed herein is assembled from its constituent nucleic acid components, purified to therapeutic-grade homogeneity, loaded with one or more gene-editing payloads into physically segregated internal compartments, decorated on its exterior surface with blood-brain-barrier-targeting ligands and stealth moieties, and finally formulated and subjected to release testing prior to administration. Each operation described below is presented in stepwise detail sufficient to permit a person of ordinary skill in the art of structural DNA nanotechnology and therapeutic nanoparticle manufacture to reproduce the manufacture of the nanorobot without undue experimentation.

The assembly approach builds upon the foundational principle of scaffolded DNA origami, in which a long single-stranded viral genome is folded into a predetermined three-dimensional geometry by the cooperative hybridization of a large set of short synthetic oligonucleotide staple strands, each of which binds to two or more spatially distinct regions of the scaffold and thereby brings those regions into defined proximity [Rothemund 2006]. The three-dimensional, multilayer, and curved-surface extensions of that principle, including honeycomb and square-lattice arrangements of parallel helices and the controlled introduction of bends and twists, enable the closed spherical cage geometry employed by the present nanorobot [Douglas 2009]. The protocols set forth herein are organized into discrete unit operations, each of which is independently characterized and controlled, so that the overall manufacturing process is modular, transferable across scales, and amenable to release on the basis of objective acceptance criteria.

### Assembly Protocol

The assembly of the folded cage proceeds through three principal unit operations performed in sequence: preparation and qualification of the single-stranded scaffold, pooling and qualification of the staple strand set, and thermal annealing of the scaffold and staple pool to drive cooperative folding into the target geometry.

### Scaffold Preparation

The scaffold strand employed is the single-stranded circular bacteriophage-derived DNA designated M13mp18, comprising 7,249 nucleotides. Single-stranded M13mp18 scaffold is produced by propagation of the phage in a suitable bacterial host, followed by isolation of phage particles from the culture supernatant and extraction of the encapsidated single-stranded genome. The extracted scaffold is purified to remove host nucleic acids, residual proteins, lipids, and endotoxin, by a combination of precipitation, chromatographic, and membrane-based steps selected to deliver material of a purity grade commensurate with the intended downstream use.

The purified scaffold is subjected to quality control by two orthogonal analytical methods. First, the integrity and size of the scaffold are confirmed by agarose gel electrophoresis, in which intact circular single-stranded M13mp18 migrates as a single dominant band of the expected apparent mobility, with the absence of low-molecular-weight smears or degradation products indicating that the scaffold has not been nicked or fragmented. Second, concentration and nucleic acid purity are determined by ultraviolet spectrophotometry, in which absorbance is measured at 260 nanometers to quantify nucleic acid concentration and the ratio of absorbance at 260 nanometers to absorbance at 280 nanometers is evaluated to confirm freedom from protein contamination, an acceptable ratio falling within the range customary for pure nucleic acid preparations.

Following qualification, the scaffold is adjusted to a working stock concentration of 100 nanomolar in folding buffer and stored at minus 20 degrees Celsius until use. Storage of the scaffold as a defined-concentration stock in the same buffer used for annealing minimizes lot-to-lot variability and avoids the introduction of incompatible salt or chelator concentrations at the annealing step. Scaffold stocks are subjected to a limited number of freeze-thaw cycles, and aliquoting is employed to preserve scaffold integrity over the qualified storage interval.

### Staple Pooling

The staple strand set comprises between 200 and 300 distinct synthetic oligonucleotide sequences, the exact number and sequence composition being dictated by the target cage geometry and the placement of functional extensions described elsewhere herein. Each staple is synthesized at a scale of between 10 and 100 nanomoles per sequence and is purified by high-performance liquid chromatography to remove truncated synthesis failure products, deletion sequences, and protecting-group adducts, since the presence of incorrect-length staples reduces folding fidelity and introduces structural heterogeneity into the finished cage.

Upon receipt of qualified individual staples, the sequences are normalized to a uniform concentration and combined in equimolar proportion to form a single working staple pool. Equimolar pooling ensures that no individual staple is present in deficit relative to its neighbors, a condition that would otherwise leave portions of the scaffold unsatisfied and promote misfolding or aggregation. The pooled staples are formulated such that, when combined with the scaffold at the annealing step, each staple is present at a final concentration of 500 nanomolar, corresponding to a staple-to-scaffold molar ratio of approximately 5 to 1.

The fivefold molar excess of each staple relative to the scaffold is selected to drive the folding reaction toward completion by mass action, ensuring that every scaffold binding site is occupied during the cooperative annealing process even in the presence of minor concentration uncertainties in individual staple stocks. The excess unincorporated staple strands remaining at the conclusion of folding are subsequently removed during purification as described below. The composition of the staple pool, including the identity and relative proportion of each constituent sequence, is recorded as a controlled formulation so that the pool may be reconstituted reproducibly across manufacturing campaigns.

### Thermal Annealing

Folding of the scaffold and staple pool into the target cage geometry is accomplished by controlled thermal annealing in a folding buffer comprising 40 millimolar Tris-HCl at pH 8.0, 20 millimolar acetic acid, 2 millimolar ethylenediaminetetraacetic acid, and 12.5 millimolar magnesium chloride. The magnesium ion concentration in this buffer is selected to screen the electrostatic repulsion between the closely packed, like-charged DNA helices of the multilayer cage, thereby stabilizing the folded structure, while the chelator and buffering components maintain a controlled ionic environment throughout the temperature program.

The combined scaffold and staple mixture is processed in a programmable thermal cycler according to a defined temperature program. The mixture is first held at 95 degrees Celsius for 5 minutes to fully denature any residual secondary structure in the scaffold and to disrupt nonspecific staple interactions, presenting an unstructured scaffold to the folding pathway. The mixture is then cooled in a stepwise ramped fashion, in which the temperature is decreased by 1 degree Celsius every 5 minutes from 95 degrees Celsius down to 60 degrees Celsius, then by 1 degree Celsius every 15 minutes from 60 degrees Celsius down to 40 degrees Celsius, and then by 1 degree Celsius every 30 minutes from 40 degrees Celsius down to 25 degrees Celsius, after which the sample is held at 4 degrees Celsius.

The progressively slowing cooling rate across successive temperature windows is deliberately structured so that the most kinetically demanding folding transitions, which occur in the intermediate and lower temperature ranges as cooperative staple incorporation locks in the cage geometry, are afforded the greatest available time for correct strand pairing and structural relaxation. The slower ramp in the lower temperature windows allows misincorporated staples to dissociate and rebind in their correct registers, favoring the thermodynamically preferred folded product over kinetically trapped misfolded species. The total duration of the annealing program is between 16 and 20 hours.

When performed as described, the thermal annealing program drives folding of the scaffold and staples into the spherical cage geometry at high assembly yield, with the majority of scaffold molecules being incorporated into correctly folded cages as confirmed by the downstream quality control methods [Rothemund 2006][Douglas 2009]. The use of a programmable thermal cycler ensures that the temperature program is executed identically across batches, and the recorded thermal profile constitutes part of the batch record for the folded intermediate.

### Purification

Following thermal annealing, the crude folding reaction contains, in addition to the correctly folded cages, a population of excess unincorporated staple strands, a minor fraction of misfolded or partially folded structures, and a minor fraction of higher-order aggregates. Three purification methods are provided, each suited to a different stage of process development and a different quality grade, and the methods may be employed alone or in combination depending upon the purity requirements of the intended use.

### Polyethylene Glycol Precipitation

For research-grade material and for rapid removal of the bulk of excess staple strands, the folded cages are purified by polyethylene glycol precipitation. To the crude folding reaction is added a precipitation solution such that the final mixture contains 8 percent polyethylene glycol of average molecular weight 8,000, 500 millimolar sodium chloride, and 13 millimolar magnesium chloride. Under these conditions the large, highly structured cages are selectively driven out of solution while the small free staple strands remain soluble. The precipitated cages are collected by centrifugation at 16,000 times gravity for 25 minutes, the supernatant containing the free staples is discarded, the pellet is washed with ethanol to remove residual polyethylene glycol and salt, and the washed cages are resuspended in a buffer appropriate to the next operation. Polyethylene glycol precipitation is rapid, scalable, and effective at removing the predominant impurity of free staples, and the procedure may be repeated to increase the depletion of unincorporated oligonucleotides.

### Size-Exclusion Chromatography

For therapeutic-grade material, the folded cages are purified by size-exclusion chromatography on a resin selected for the separation range encompassing the cage and its principal impurities, such as a cross-linked allyl dextran resin of the Sephacryl S-400 type or an agarose-based resin of the Sepharose CL-4B type. In this method the folding reaction is applied to a column of the resin and eluted isocratically, whereupon the large folded cages, being excluded from the pores of the resin, elute earlier than the much smaller free staple strands, which penetrate the resin pores and are thereby retarded. Higher-order aggregates, being still larger than the monomeric cage, elute in advance of the cage and are likewise resolved. The chromatographic separation therefore removes both free staples and aggregates in a single operation, delivering a monodisperse population of correctly folded cages, and the gentle, additive-free nature of the separation preserves cage integrity and avoids the introduction of precipitants.

### Rate-Zonal Centrifugation

For the highest purity and for processes intended to be compatible with good manufacturing practice, the folded cages are purified by rate-zonal centrifugation through a density gradient. A continuous gradient of 10 to 40 percent glycerol is prepared, the folding reaction is layered atop the gradient, and the gradient is subjected to ultracentrifugation for approximately 48 hours at 100,000 times gravity. Under these conditions the components of the reaction migrate through the gradient at rates determined by their size and density, so that the monomeric correctly folded cages form a discrete band well separated from the slower-migrating free staples and the faster-migrating aggregates and multimers. Because separation is achieved on the basis of both size and density, rate-zonal centrifugation resolves species that are difficult to distinguish by size alone, and it therefore delivers the highest purity of the three methods. The defined-gradient, additive-controlled nature of the operation renders it compatible with manufacture under good manufacturing practice, and the fractionated cage band is collected for subsequent loading or formulation.

### Quality Control of the Folded Cage

The purified folded cage is qualified prior to cargo loading by a panel of orthogonal analytical methods that together confirm correct folding, correct dimensions, correct three-dimensional structure, and acceptable size distribution.

Correct folding and monodispersity are assessed by agarose gel electrophoresis, in which a correctly and homogeneously folded cage population migrates as a single sharp band of defined mobility. A single band confirms that the population consists predominantly of the intended folded species, whereas the appearance of slower-migrating bands indicates aggregation or multimerization and the appearance of faster-migrating or smeared material indicates misfolding, incomplete folding, or residual free staples. The electrophoretic mobility of the folded cage is compared against that of the unfolded scaffold to confirm the expected compaction upon folding.

The physical dimensions of individual cages are confirmed by atomic force microscopy, in which individual particles deposited on a flat substrate are imaged at nanometer resolution to measure their lateral dimensions. Acceptance requires that the measured dimensions of the cage fall within plus or minus 10 percent of the design dimensions, and that the proportion of malformed particles observed in the imaged field be less than 5 percent. Atomic force microscopy thereby provides a direct, particle-by-particle assessment of dimensional fidelity and folding quality.

The three-dimensional architecture of the cage is confirmed by transmission electron microscopy with negative staining, in which the particles are embedded in a heavy-metal stain that delineates their outline and internal contrast and are imaged in projection. Negative-stain transmission electron microscopy confirms that the particles exhibit the intended spherical, closed, hollow architecture and the expected diameter, and class averaging of many particle images further confirms the reproducibility of the folded three-dimensional structure across the population.

The hydrodynamic size and size distribution of the cages in solution are determined by dynamic light scattering, which reports the hydrodynamic diameter of the particles and the polydispersity index of the population. Acceptance requires a polydispersity index target of less than 0.2, indicating a narrow, monodisperse size distribution consistent with a homogeneous population of correctly folded monomeric cages. The hydrodynamic diameter measured by dynamic light scattering is expected to fall within the spherical cage size range of 100 to 140 nanometers, accounting for the hydration shell and any surface extensions present at the time of measurement.

### Cargo Loading Methods

The interior of the folded cage is organized into discrete internal compartments, each of which is loaded independently with its respective gene-editing payload so that the distinct editors remain physically separated within the assembled nanorobot. Three orthogonal loading methods are provided, and the method selected for a given compartment is matched to the chemistry of the editor to be loaded and to the purity grade required.

### Oligonucleotide-Mediated Conjugation

In the first method, the gene-editing protein is expressed as a fusion bearing a terminal self-labeling or covalent-capture tag, such as a HaloTag, a SNAP-tag, or a SpyTag. A ligand specific to the tag, such as a chloroalkane ligand for HaloTag, a benzylguanine ligand for SNAP-tag, or a SpyCatcher partner for SpyTag, is chemically conjugated to a synthetic oligonucleotide of between 20 and 30 nucleotides in length whose sequence is complementary to interior capture strands that are presented within the target compartment of the cage during folding. The tagged editor is thereby coupled to the capture oligonucleotide through the tag-ligand reaction, and the editor-oligonucleotide conjugate is then captured within the compartment by hybridization of the conjugated oligonucleotide to the complementary interior capture strands. Hybridization is performed at 4 degrees Celsius overnight to favor specific, complete strand pairing and to minimize nonspecific association. Excess unbound editor and unhybridized conjugate are removed by polyethylene glycol precipitation or by ultrafiltration, and the method achieves loading efficiencies of between 70 and 95 percent.

### Biotin-Streptavidin Capture

In the second method, the guide RNA component of the editing ribonucleoprotein is synthesized or modified to bear a biotin moiety, and the editing ribonucleoprotein is assembled by combining the biotinylated guide RNA with its cognate editor protein. The target compartment of the cage presents streptavidin-binding staples, such that the biotinylated ribonucleoprotein is captured within the compartment through the biotin-streptavidin interaction, which exhibits an extremely high affinity characterized by a dissociation constant of approximately 1×10⁻¹⁴ molar. The exceptionally tight, effectively irreversible nature of this interaction ensures that, once captured, the ribonucleoprotein is retained within the compartment under physiological and storage conditions. Unbound ribonucleoprotein is removed by washing, and the method achieves loading efficiencies of between 75 and 90 percent.

### Copper-Free Click Chemistry

In the third method, the editor is modified to bear an azide functional group and the interior capture strands of the target compartment are modified to bear a dibenzocyclooctyne group. Upon contact, the azide and dibenzocyclooctyne undergo a strain-promoted, copper-free azide-alkyne cycloaddition that forms a stable covalent linkage between the editor and the capture strand without the need for a copper catalyst, thereby avoiding copper-mediated damage to the nucleic acid components. The resulting covalent attachment renders the loading effectively permanent, and the method achieves loading efficiencies greater than 85 percent. This copper-free click chemistry is a bioorthogonal conjugation chemistry of established regulatory relevance, having been employed in approved antibody-drug conjugate manufacture, which supports its suitability for therapeutic nanoparticle production.

### Per-Class Loading Targets and Loading Quality Control

The loading methods above are applied to deliver the specific classes of editors carried by the nanorobot, and loading-efficiency targets are established for each class. For base editors, the loading-efficiency target is between 80 and 95 percent. For prime editors, the loading-efficiency target is between 75 and 90 percent. For Cas13-class effectors, the loading-efficiency target is between 85 and 95 percent. These per-class targets account for differences in the size, charge, and tag compatibility of the respective editors and establish the acceptance basis for the loading operation.

Loading is verified by a panel of orthogonal assays. The presence and quantity of the captured editor are confirmed by Western blot against the editor protein, which establishes both the identity of the loaded species and the amount loaded per cage. The extent of loading is further quantified by a fluorescence assay, in which a fluorescent reporter associated with the editor or the capture chemistry reports the fraction of compartments occupied. The functional integrity of the loaded editor is confirmed by a cell-based activity assay, in which the loaded nanorobot or the released editor is shown to retain its intended editing activity in cells, thereby demonstrating that the loading process has not impaired catalytic function.

Each compartment of the cage is loaded independently with its respective editor in a dedicated loading operation, and the loading chemistries are selected so that the capture of one editor in its compartment does not cross-react with the capture strands of another compartment. As a consequence, the distinct editors carried by the nanorobot remain physically separated within their respective compartments throughout loading, purification, formulation, and storage, which preserves the orthogonality of the multiple editing functions and prevents premature interaction among the payloads.

### Surface Functionalization

The exterior surface of the loaded cage is functionalized to confer blood-brain-barrier-targeting capability and to confer stealth properties that prolong circulation and reduce nonspecific interactions. Both functionalities are introduced through staple-based display, such that the targeting ligands and stealth moieties are presented at defined positions and densities established by the design of the staple set.

### Transferrin Receptor Aptamer Attachment

The cage is decorated with between 18 and 30 transferrin-receptor-binding aptamers to mediate receptor-mediated transcytosis across the blood-brain barrier. Each aptamer is displayed as an extension of a staple strand, the extended staple comprising, in sequence from its anchoring core, a core staple segment of approximately 32 nucleotides that hybridizes to the scaffold as part of the cage structure, a poly-thymidine linker of approximately 10 nucleotides that projects the aptamer away from the cage surface and confers conformational freedom, and an aptamer segment of approximately 40 nucleotides that folds into the transferrin-receptor-binding structure. Because the extended staples are incorporated during the folding reaction itself, the aptamers are positioned at predetermined locations across the cage surface, yielding a uniform and multivalent distribution of targeting ligands that supports avid engagement of transferrin receptors.

The transferrin-receptor binding of the functionalized cage is validated by two orthogonal methods. Binding affinity is measured by surface plasmon resonance, which reports a dissociation constant in the range of approximately 10 to 50 nanomolar for the interaction between the displayed aptamers and immobilized transferrin receptor, confirming that the multivalent display achieves high-avidity binding. Cellular target engagement is confirmed by flow cytometry on transferrin-receptor-positive cells, which demonstrates selective association of the functionalized cage with cells bearing the receptor [Li 2020][Yu 2011].

### PEGylation

The cage surface is further functionalized with polyethylene glycol to confer stealth properties. Stealth is introduced through cholesterol-anchored polyethylene glycol staples bearing polyethylene glycol of molecular weight 2,000 or 5,000, incorporated at a surface density of between 5 and 10 percent of the available staple positions. The cholesterol moiety anchors the construct within the duplex regions of the cage by hydrophobic insertion, while the polyethylene glycol chain extends outward to form a hydrated stealth corona over the cage surface.

The polyethylene glycol corona confers several benefits to the circulating nanorobot. It extends the circulation half-life from approximately 1 hour, characteristic of an unmodified cage that is rapidly cleared, to approximately 6 to 12 hours, by sterically shielding the particle from clearance mechanisms. It reduces the formation of a serum protein corona on the particle surface, thereby preserving the accessibility of the displayed targeting aptamers. And it minimizes nonspecific cellular uptake, directing the particle toward receptor-mediated transcytosis at the blood-brain barrier rather than nonspecific sequestration. The selection of the polyethylene glycol molecular weight and surface density balances the magnitude of the stealth benefit against the preservation of targeting-ligand accessibility. In alternative embodiments, the polyethylene glycol stealth layer may be supplemented or replaced by a zwitterionic-polymer coating, such as a poly(carboxybetaine) or poly(sulfobetaine) layer conjugated to staple strands, which resists protein adsorption and non-specific uptake through strong surface hydration; accordingly, cholesterol-anchored polyethylene glycol, zwitterionic polymers, and combinations thereof are each contemplated as the outer stealth coating of the nanostructure.

### Final Purification and Formulation

Following loading and surface functionalization, the fully assembled and loaded nanorobot is subjected to a final purification to isolate the monodisperse, fully assembled and fully loaded product from any incompletely loaded, incompletely functionalized, or aggregated species. This final purification is performed by rate-zonal centrifugation through a glycerol density gradient as described above, in which the monodisperse, fully assembled and loaded peak is collected as a discrete band, the target representing greater than 95 percent of the collected material and excluding aggregates and incomplete constructs that migrate differently on the gradient.

The purified product is formulated into the final drug-product buffer comprising 50 millimolar Tris-HCl at pH 7.4, 5 millimolar magnesium chloride, 150 millimolar sodium chloride, 0.01 percent Tween-20, and 5 to 10 percent trehalose. The magnesium chloride maintains the structural integrity of the multilayer cage, the sodium chloride establishes physiological ionic strength, the Tween-20 surfactant minimizes surface adsorption and aggregation, and the trehalose serves as a cryoprotectant and lyoprotectant that preserves the structure during freezing or drying. The product is formulated to a final concentration of between 0.5 and 5 micromolar.

The formulated drug product is stored at minus 80 degrees Celsius, under which conditions it is stable for at least 24 months, or alternatively is lyophilized for storage at room temperature, under which condition it is stable for at least 12 months when formulated with 20 percent trehalose as the lyoprotectant. The intended administration of the product is at a dose of between 1 and 10 milligrams per kilogram of body weight, the formulation concentration being selected to deliver this dose in an acceptable administration volume.

The finished drug product is released for use against a defined panel of release tests addressing identity, purity, potency, safety, and stability. Identity is confirmed by nucleic acid sequencing of the constituent strands, by liquid chromatography coupled to tandem mass spectrometry of the loaded protein editors, and by transmission electron microscopy and atomic force microscopy confirming the expected three-dimensional structure and dimensions. Purity is required to exceed 95 percent monomeric product, with less than 5 percent free staple strands and an endotoxin level below 0.1 endotoxin units per milliliter.

Potency is established against editor-class-specific thresholds, requiring base editing activity of at least 60 percent, prime editing activity of at least 40 percent, and Cas13 activity of at least 60 percent in the relevant activity assays, together with transferrin-receptor binding characterized by a dissociation constant in the range of 5 to 100 nanomolar. Safety is established by sterility testing, by mycoplasma testing, and by demonstration that off-target editing activity is below 0.1 percent. Stability is monitored across the storage interval against acceptance criteria requiring that the formulation pH remain within the range of 7.2 to 7.6, that the particle size remain within the range of 90 to 160 nanometers, that the polydispersity index remain below 0.25, and that activity retention be at least 85 percent at the 6-month time point, thereby confirming that the product retains its identity, structure, and function over the qualified shelf life [Huang 2024][Tam 2020].

### Base Editing Systems

The first editing modality carried by the present nanorobot system is base editing, a class of programmable enzymes capable of installing single-nucleotide changes in genomic DNA without generating a double-strand break and without requiring a donor DNA template. The platform incorporates two complementary base editor families: adenine base editors (ABE) and cytosine base editors (CBE). Together these two families address the large majority of pathogenic point mutations relevant to neurological disease, in which a single disease-causing nucleotide substitution can be reverted to the wild-type identity by direct chemical conversion of one base into another.

Adenine base editors used in the present system include ABE7.10, ABE8e, and ABE9e. ABE7.10 represents the originally engineered, laboratory-evolved adenine base editor that established the feasibility of programmable adenine-to-guanine conversion in genomic DNA [Gaudelli 2017]. ABE 8e is a substantially accelerated variant derived through additional rounds of directed evolution that markedly increases deaminase catalytic rate, expanding both editing efficiency and the practical editing window while retaining the core architecture [Richter 2020]. ABE9e is a further refined adenine base editor selected within the present platform for its narrowed editing window and high on-target precision, properties that are particularly advantageous in the post-mitotic, non-dividing neurons that constitute the principal therapeutic targets of this system.

The mechanism of adenine base editing proceeds through a defined sequence of molecular events. A single guide RNA (sgRNA) directs the base editor, which comprises a catalytically impaired Cas9 nickase fused to an engineered adenine deaminase domain, to a genomic locus complementary to the sgRNA spacer. Upon target engagement, the protein-RNA complex unwinds the local DNA duplex and forms an R-loop, in which the guide RNA base-pairs with the target strand and displaces the non-target strand as a transient stretch of single-stranded DNA. The engineered TadA deaminase domain accesses the exposed single-stranded non-target strand and chemically deaminates a target adenine, converting it to inosine. Because cellular polymerases and the cellular replication and repair machinery read inosine as guanine, the edited position is interpreted as guanine. Nicking of the unedited strand by the Cas9 nickase biases the cell toward using the edited strand as the template during repair, so that the original A:T base pair is permanently converted to a G:C base pair [Gaudelli 2017][Richter 2020].

Cytosine base editors used in the present system include BE4max, CBE4max, and AncBE4max. These editors are codon-and architecture-optimized cytosine base editors engineered for high expression and high editing efficiency in mammalian cells, with AncBE4max employing an ancestrally reconstructed deaminase to further enhance activity and product purity [Koblan 2018]. The foundational cytosine base editing chemistry on which these optimized constructs are built was first demonstrated as a programmable cytosine-to-thymine conversion system [Komor 2016].

The mechanism of cytosine base editing parallels that of adenine base editing but operates on a different base. An sgRNA directs a Cas9 nickase fused to a cytidine deaminase, typically an APOBEC-family enzyme, to the target locus, where R-loop formation again exposes the non-target strand as single-stranded DNA. The APOBEC deaminase converts a target cytosine to uracil. A uracil glycosylase inhibitor domain, fused to or co-delivered with the editor, blocks the cellular base-excision repair enzyme uracil-DNA glycosylase from removing the newly formed uracil, thereby preserving the intermediate long enough for it to be fixed. The uracil is then read and ultimately replicated as thymine, and nicking of the opposite strand biases repair toward the edited strand, so that the original C: G base pair is permanently converted to a T:A base pair [Komor 2016][Koblan 2018].

For the present platform, ABE9e is the preferred adenine base editor for correction in post-mitotic neurons. Post-mitotic neurons do not divide, so editing approaches that depend on cell division to resolve or propagate an edit are poorly suited to the central nervous system. Base editing does not require homology-directed repair or active DNA replication to install its change, which makes it well matched to the non-dividing neuronal context, and the narrowed editing window and high precision of ABE9e reduce the likelihood of unintended bystander edits at adjacent adenines within the protospacer.

The human safety and tolerability of base editing as a therapeutic modality is supported by publicly reported clinical programs employing base editing in human subjects, which have advanced base editing approaches into the clinic. These publicly reported clinical programs indicate that base editors have been administered to human subjects and are under active clinical evaluation, supporting the translational rationale for incorporating base editing into the present system.

Within the nanorobot architecture, the base editor is integrated together with its sgRNA as the cargo of Compartment 1. The base editor cargo, at approximately 180 to 200 kDa, is dimensionally matched to the 30×30×40 nm internal volume of Compartment 1. Compartment 1 is configured to release first, deploying the base editor at the outset of the therapeutic sequence to effect permanent, single-nucleotide correction of the targeted genomic locus before the subsequent editing modalities are released.

### Prime Editing Systems

The second editing modality carried by the present nanorobot system is prime editing, a versatile genome editing approach that writes new genetic information directly into a specified DNA locus without requiring double-strand breaks or donor DNA templates. Whereas base editing is limited to the specific transition chemistries achievable by deamination, prime editing can install all twelve possible single-base substitutions as well as small insertions and small deletions, substantially broadening the range of pathogenic variants that can be corrected. The platform incorporates prime editing variants designated PE2, PE3, PE3b, PE5, and PEmax.

The mechanism of prime editing is fundamentally different from that of base editing. A prime editing guide RNA (pegRNA), which carries both a spacer that defines the target site and an extension encoding the desired edit, directs a Cas9 nickase fused to an engineered reverse transcriptase to the target locus. The nickase cleaves the non-target strand, exposing a free 3′ DNA end. The primer-binding site contained within the pegRNA extension hybridizes to this exposed 3′ end. The fused reverse transcriptase then extends the DNA 3′ end using the reverse transcriptase template portion of the pegRNA as a template, directly polymerizing the encoded edit into a newly synthesized 3′ flap. The cell resolves the resulting 3′ flap, which contains the intended edit, through its endogenous DNA repair pathways, and the edited sequence is incorporated into the genome. By this templated copy-and-paste chemistry, prime editing achieves precise insertions, deletions, and substitutions specified entirely by the pegRNA [Anzalone 2019].

The prime editing variants differ in the auxiliary components used to enhance editing outcomes. PE2 employs the prime editor protein together with the pegRNA alone. PE3 adds a separate nicking sgRNA that nicks the non-edited strand to bias repair toward retention of the edit, increasing efficiency. PE3b is a refinement of this strategy in which the nicking sgRNA is designed to nick only after the edit has been installed, reducing the incidence of unwanted insertions and deletions arising from concurrent nicks on both strands. PE5 further incorporates transient modulation of the cellular mismatch repair system through a dominant-negative MLH1 protein (MLH1dn), which transiently suppresses mismatch repair-mediated reversal of the intended edit and thereby yields the highest editing efficiency together with the lowest frequency of unwanted insertions and deletions. PEmax denotes a codon-optimized and architecturally optimized prime editor protein that improves expression and activity, and PE5 is typically deployed in combination with the PEmax architecture [Chen 2021].

The capabilities of prime editing make it especially valuable as a complement to base editing within the present platform. Prime editing can install all twelve base-to-base substitutions, including transversions that base editors cannot directly produce; it can introduce templated insertions of up to approximately 80 base pairs; and it can introduce templated deletions of up to approximately 80 base pairs. Taken together, these capabilities allow prime editing to address a large fraction of known pathogenic variants, including small insertions, small deletions, and complex substitutions that fall outside the reach of base editors, thereby substantially extending the corrective scope of the system.

The human feasibility of prime editing as a therapeutic modality is supported by publicly reported prime-editing clinical programs that have advanced prime editing toward and into human application. These publicly reported clinical programs provide translational support for incorporating prime editing as a deployable editing modality within the present nanorobot system.

Within the nanorobot architecture, the prime editing system is integrated as the cargo of Compartment 2, comprising the PE5 prime editor, its pegRNA, an optional nicking sgRNA, and the MLH1dn mismatch-repair modulator. The prime editor cargo, at approximately 220 to 250 kDa, is dimensionally matched to the 30×30×45 nm internal volume of Compartment 2. Compartment 2 is configured to release second, deploying the prime editor approximately 4 to 8 hours after the base editor of Compartment 1, so that the broader corrective capabilities of prime editing follow the initial single-nucleotide base editing in a defined temporal sequence.

### Cas13 RNA Editing Systems

The third editing modality carried by the present nanorobot system operates at the level of RNA rather than DNA, using the Cas13 family of RNA-guided, RNA-targeting enzymes. Unlike base editing and prime editing, which install permanent changes in the genome, Cas13-based editing acts on messenger RNA and is therefore reversible, dose-titratable, and free of any permanent alteration to the genomic sequence. The platform incorporates members of the Cas13 family including Cas13a, exemplified by LshCas13a; Cas13b, exemplified by PspCas13b; Cas13d, exemplified by CasRx, which is a compact effector of approximately 930 amino acids; and the Cas13Y effector together with its high-fidelity engineered counterpart hfCas13Y. The compactness of Cas13d (CasRx) is particularly advantageous for packaging and delivery within a size-constrained nanorobot compartment.

Cas13 enzymes can be employed for targeted knockdown of a specified transcript. In the knockdown mechanism, a CRISPR RNA (crRNA) guides the Cas13 effector to a complementary target messenger RNA. Hybridization of the crRNA with the target mRNA activates the two HEPN ribonuclease domains of the Cas13 effector. The activated ribonuclease domains cleave the target mRNA, reducing the abundance of the encoded protein. Depending on the target and the design of the crRNA, this knockdown approach can lower the level of the targeted protein by approximately 50 to 95 percent, providing a programmable means of reducing the expression of a deleterious or overexpressed gene product [Abudayyeh 2017][Konermann 2018]. It is recognized that, upon target recognition, certain Cas13 effectors can exhibit collateral, non-specific ribonuclease activity against bystander transcripts; the present system is configured to mitigate this consideration through the use of high-fidelity Cas13 variants such as hfCas13Y, through the compartmentalized and logic-gated release that confines effector activity to the intended cell population and to a defined temporal window, and through dose-titratable crRNA selection [Konermann 2018].

Cas13 effectors can also be configured for reversible RNA base editing rather than transcript destruction. In this configuration, a catalytically inactive Cas13 effector (dCas13), in which the HEPN ribonuclease activity has been abolished, is fused to an adenosine deaminase acting on RNA, specifically the ADAR2 deaminase domain. The crRNA positions the dCas13-ADAR2 fusion at a defined site on the target transcript, and the ADAR2 domain performs programmable adenosine-to-inosine editing of the RNA; inosine is read by the cellular machinery as guanosine, so that an adenosine is functionally converted to a guanosine at the RNA level. This foundational dCas13-ADAR2 approach for programmable adenosine-to-inosine RNA editing, designated REPAIR (RNA editing for programmable A-to-I replacement), installs corrections at the transcript level that are not written into the genome and that decay as the edited transcripts turn over [Cox 2017]. Engineered variants of this general approach further extend the chemistry to cytosine-to-uridine RNA editing in related platforms, broadening the range of transcript-level corrections available without genomic alteration.

The advantages of Cas13-based RNA editing are complementary to those of the DNA-level modalities. Because Cas13 acts on RNA, its effects are inherently reversible: when the Cas13 system is no longer present, transcript levels and sequences return to their baseline state as new RNA is transcribed from the unaltered genome. The magnitude of the effect is dose-titratable, allowing the degree of knockdown or RNA editing to be tuned by adjusting the amount of effector delivered. No permanent change is made to the DNA, which is advantageous when the therapeutic goal is to fine-tune protein levels, to provide an adjustable degree of correction, or to avoid committing the cell to an irreversible genomic edit.

The human central nervous system feasibility of Cas13-based therapeutics is supported by publicly reported Cas13 clinical programs, including programs employing intracerebroventricular delivery for direct administration to the brain. These publicly reported clinical programs provide translational support for deploying Cas13 RNA editing within the central nervous system as part of the present nanorobot system.

Within the nanorobot architecture, the Cas13 system is integrated as the cargo of Compartment 3, comprising a Cas13d (CasRx) effector together with a crRNA array that can direct the effector to one or more target transcripts. The Cas13 cargo, at approximately 130 to 150 kDa, is dimensionally matched to the 28×28×35 nm internal volume of Compartment 3. Compartment 3 is configured to release last, deploying the Cas13 system approximately 8 to 12 hours after the prime editor of Compartment 2, so that reversible RNA-level regulation follows the permanent DNA-level corrections in a defined temporal sequence.

### Rationale for Combining Permanent and Reversible Editing

The combination within a single nanorobot of permanent genomic correction through base and prime editing with reversible, titratable RNA-level regulation through Cas13 provides a therapeutic capability that no single editing modality can achieve on its own. Permanent correction by base editing and prime editing addresses the underlying genetic lesion directly and durably, installing a one-time change that persists for the life of the edited cell and is propagated through any subsequent cell divisions, which is especially valuable in long-lived, non-dividing neurons where a single durable correction can confer lasting benefit.

Reversible regulation by Cas13 contributes a different and complementary set of properties. RNA-level knockdown and RNA base editing can be tuned in magnitude and are inherently reversible, allowing the degree of protein reduction or transcript correction to be adjusted and, if necessary, allowed to decay. This makes Cas13 well suited to situations in which permanent silencing or permanent correction would be undesirable, in which the optimal level of a protein is uncertain and benefits from titration, or in which ongoing, adjustable control of gene expression is therapeutically preferable to an irreversible change.

Pairing these modalities within one temporally sequenced delivery system confers therapeutic redundancy and ongoing control. If a permanent edit installed by the base editor or prime editor achieves only partial correction at the population level, the Cas13 system can provide additional fine-tuning of the resulting protein level at the RNA layer. Conversely, the durable genomic corrections provide a stable therapeutic foundation that does not depend on continued presence of the editing machinery. The defined release sequence, in which permanent DNA-level editing precedes reversible RNA-level regulation, allows the system first to establish a durable genomic correction and then to apply adjustable, reversible control over residual protein expression. The result is a multilayered therapeutic strategy that combines the permanence and durability of genomic editing with the reversibility, titratability, and ongoing controllability of RNA editing, offering a degree of redundancy and adjustable control that is not available from any single editing modality deployed alone.

### Structure and Challenge of the Blood-Brain Barrier

The blood-brain barrier is the principal physiological obstacle that has historically frustrated the systemic delivery of therapeutic agents to the central nervous system, and an appreciation of its molecular architecture is essential to understanding both the problem the present invention solves and the manner in which it solves it. The barrier is formed at the level of the cerebral microvasculature by a continuous monolayer of specialized brain microvascular endothelial cells that line the lumen of the capillaries perfusing the brain parenchyma. Unlike the fenestrated or discontinuous endothelium found in peripheral capillary beds, the endothelium of the cerebral vasculature is sealed along its intercellular clefts by an elaborate and densely organized network of tight junctions. These junctions are constituted by integral membrane proteins, including the tetraspanning protein occludin and the claudin family of proteins, of which claudin-5 is the predominant and functionally dominant species at the brain endothelium. The transmembrane junctional proteins are anchored intracellularly to the actin cytoskeleton through a family of cytoplasmic scaffolding proteins, of which zonula occludens-1, commonly designated ZO-1, is a central member. The cooperative assembly of occludin, claudin-5, and ZO-1, together with associated junctional adhesion molecules, produces a paracellular seal of extraordinarily high electrical resistance that effectively eliminates the diffusional pathway between adjacent endothelial cells through which small hydrophilic solutes might otherwise pass.

Beyond the occlusion of the paracellular route, the brain endothelium is distinguished by a markedly suppressed rate of constitutive vesicular transport across the cell body. In peripheral endothelia, fluid-phase and adsorptive transcytosis proceed at appreciable rates, permitting macromolecules to traverse the cell interior within membrane-bounded vesicles. At the brain endothelium, by contrast, this nonspecific transcellular trafficking is constitutively repressed to a minimal level, such that bulk-phase pinocytosis contributes negligibly to the passage of circulating macromolecules. The consequence is that neither the space between the cells nor the body of the cells offers a meaningful default route by which a blood-borne agent may reach the brain.

The barrier is further reinforced by an array of energy-dependent efflux transporters that are expressed at high density on the luminal, blood-facing membrane of the endothelial cells. Foremost among these is P-glycoprotein, an adenosine-triphosphate-binding cassette transporter of broad substrate specificity that actively recognizes and expels a vast structural range of lipophilic and amphipathic molecules back into the bloodstream before they can complete their passage through the cell. Acting in concert with P-glycoprotein is the breast cancer resistance protein, designated BCRP, a second efflux pump of complementary and partially overlapping substrate range. Together these transporters impose an active, vectorial barrier that defeats many agents which might otherwise possess the physicochemical properties to diffuse across the endothelial membranes, because the very lipophilicity that would favor membrane partitioning frequently renders a molecule a substrate for efflux.

The endothelial barrier does not act in isolation but is embedded within and regulated by the larger neurovascular unit. The abluminal surface of the capillary is invested by pericytes, contractile mural cells that share a basement membrane with the endothelium and that contribute to the induction and maintenance of barrier properties and to the regulation of capillary tone and microvascular stability. Surrounding this assembly, the terminal processes of astrocytes, known as astrocyte end-feet, ensheath nearly the entire abluminal surface of the cerebral microvessels. These end-feet provide instructive signals that induce and sustain the specialized barrier phenotype of the endothelium and complete the multicellular architecture that renders the brain a privileged compartment. The integrated action of the tight-junctional seal, the suppression of transcytosis, the efflux transporters, and the surrounding pericytes and astrocytic end-feet thus produces a barrier whose selectivity is among the most stringent encountered anywhere in the body.

The quantitative consequence of this architecture is severe. As a general matter, fewer than approximately two percent of small-molecule drug candidates possess the combination of low molecular weight, suitable lipophilicity, minimal hydrogen-bonding burden, and resistance to efflux that permits any clinically meaningful crossing of the intact barrier, and the overwhelming majority of small molecules are therefore excluded. For large molecules, including therapeutic proteins, antibodies, nucleic acids, and the multiprotein genome-editing complexes that are central to the present invention, the situation is far more restrictive still, with fewer than approximately one-tenth of one percent of administered large molecules crossing the intact barrier by natural means. A delivery vehicle that is large, that bears nucleic acid and protein cargo, and that must remain intact in transit therefore confronts the most adverse end of this exclusion spectrum.

This pharmacological reality has dictated the prevailing approach to delivering genome-editing therapeutics to the central nervous system. Because systemic administration cannot deliver an effective dose of editing machinery across the intact barrier, clinical and preclinical genome-editing programs directed at neurological targets have relied predominantly on invasive, direct administration that bypasses the barrier altogether. Such approaches include intracerebroventricular infusion, in which the editing agent is introduced directly into the cerebrospinal fluid of the cerebral ventricles, and intraparenchymal injection, including intrastriatal administration, in which the agent is deposited directly into the brain tissue through a surgically placed cannula. Publicly reported neurological genome-editing programs that pursue direct brain delivery illustrate the reliance on such surgical routes of administration to place editing machinery within the central nervous system. While these direct routes can place an active agent within the brain, they require neurosurgical intervention, carry attendant procedural risk, achieve only localized distribution about the site of administration, and are poorly suited to repeated dosing or to the treatment of diffuse or widely distributed neurological disease. The persistence of these invasive routes as the standard of practice establishes a clear and unmet need for a noninvasive, systemically administered platform capable of delivering intact genome-editing cargo across the blood-brain barrier, which need the present blood-brain barrier crossing subsystem is configured to meet.

### Validated DNA-Nanostructure BBB Crossing

The blood-brain barrier crossing subsystem of the present invention does not rest upon a speculative premise but is grounded in a body of validated experimental observation establishing that three-dimensional DNA nanostructures of appropriate size and geometry are capable of crossing the blood-brain barrier. It has been demonstrated in vivo that spherical, three-dimensional DNA nanocages can traverse the intact blood-brain barrier and accumulate within brain tissue following systemic administration, with passage occurring through an endocytic, vesicle-mediated mechanism rather than through any disruption of the barrier itself [Tam 2020]. Of particular significance to the present design, that work established that the geometry of the DNA nanostructure is a determinant of crossing efficiency, with spherical architectures outperforming tubular or elongated architectures of comparable composition in their ability to reach the brain. This finding directly informs the selection of an assembled spherical cage geometry for the present platform.

The capacity of framework nucleic acid nanostructures to traverse the barrier has been independently corroborated in defined barrier models. Framework-DNA nanodevices have been shown to cross an artificial blood-brain barrier model constructed from cultured barrier-forming cells, and the crossing in that system was found to proceed by a transcytotic route that is substantially independent of the lysosomal degradation pathway, such that the nanostructure is transported across the cell interior and released on the far side rather than being trafficked to lysosomes for destruction [Chen 2022]. Notably, that work observed that larger and mechanically softer framework structures penetrated the barrier more effectively than smaller or more rigid counterparts, a relationship consistent with a transcytotic mechanism that accommodates deformable cargo and that further supports the use of a structurally defined nucleic acid cage in the present platform.

The therapeutic relevance of DNA nanostructures to neurological application has likewise been demonstrated. DNA tetrahedral nanocages have been employed to deliver a neuroactive payload of relevance to neurological disorders, establishing that nucleic acid nanocages can serve not merely as passive markers of crossing but as functional carriers that convey a biologically active cargo in a manner pertinent to disease of the central nervous system [Singh 2024]. Taken together with the foregoing, this body of work confirms that the DNA nanocage is a delivery modality with demonstrated reach into the brain and demonstrated payload-carrying capability.

A synthesis of these validated observations yields a critical design principle that the present platform embodies. The collective evidence establishes that DNA nanostructures of roughly eighty to one hundred fifty nanometers in characteristic dimension, when configured with a spherical rather than an elongated geometry, are capable of crossing the blood-brain barrier and accumulating in brain tissue. The assembled spherical cage of the present invention possesses a characteristic dimension of approximately one hundred to one hundred forty nanometers and a spherical three-dimensional architecture, and therefore falls squarely within the experimentally validated window of size and geometry. The blood-brain barrier crossing subsystem is consequently not a hypothetical proposition but a deliberate engineering of the platform into a parameter space already shown to support the crossing of intact DNA nanostructures into the brain, thereby providing a sound and enabling basis for the noninvasive systemic delivery the invention contemplates.

### Active Targeting via the Transferrin Receptor

While the size and geometry of the spherical cage place it within the validated window for passive nanostructure crossing, the present platform supplements this baseline capacity with an active, receptor-mediated targeting strategy directed at the transferrin receptor, thereby substantially enhancing the rate and efficiency of brain accumulation. The transferrin receptor is a cell-surface glycoprotein that is expressed at unusually high density on the luminal membrane of the brain microvascular endothelium, where its abundance has been reported to exceed that found on most peripheral tissues by approximately ten to twenty fold. The physiological function of this receptor is to mediate the receptor-mediated transcytosis of iron-laden transferrin from the blood across the endothelium and into the brain, supplying the central nervous system with the iron required for its metabolic functions. The transferrin receptor therefore constitutes a natural, constitutively operating portal for the vectorial transport of macromolecular cargo from the bloodstream into the brain, and the recruitment of this endogenous transport pathway is among the most rational strategies available for noninvasive brain delivery.

The present platform engages this pathway by displaying, on the exterior surface of the spherical cage, a plurality of DNA aptamers selected to bind the transferrin receptor with high affinity and specificity. The use of transferrin-receptor-binding nucleic acid aptamers as agents of brain delivery is supported by direct experimental demonstration. A bifunctional aptamer configured to bind the transferrin receptor has been shown to enhance penetration across the blood-brain barrier in vivo and to trigger receptor-mediated transcytosis of its associated cargo, and importantly to do so by binding the receptor at a site that does not block the binding of endogenous transferrin, such that the natural iron-transport function of the receptor is preserved and the engineered cargo is ferried alongside rather than in competition with the receptor's physiological ligand [Li 2020]. This non-competitive mode of engagement is advantageous because it avoids both the saturation of the pathway by endogenous transferrin and any interference with normal iron homeostasis.

The principle that transferrin-receptor engagement can carry macromolecular cargo across the barrier, and that the brain uptake achieved thereby is sensitive to the affinity of the engaging ligand, is further supported in the art. It has been shown that antibody-mediated engagement of the transferrin receptor can ferry a macromolecular payload across the blood-brain barrier by receptor-mediated transcytosis, and that the magnitude of brain uptake can be tuned by adjusting the binding affinity of the receptor-engaging moiety, with appropriately moderated affinity favoring release on the brain side and thereby improving net delivery rather than trapping the cargo at the luminal surface [Yu 2011]. These teachings establish both that the transferrin receptor is a competent vehicle for transcytosis of large cargo and that affinity is a tunable parameter that may be optimized to maximize brain accumulation, considerations that inform the configuration of the aptamer display in the present platform.

In the platform of the present invention, the transferrin-receptor targeting function is realized by the integration of approximately eighteen to thirty transferrin-receptor aptamers onto the exterior of the assembled spherical cage, with the claimed range of aptamer copy number extending more broadly from approximately twelve to forty aptamers to accommodate the optimization of valency for a given application. The aptamers are distributed substantially uniformly over the surface of the cage so as to present a multivalent array of binding moieties that engages a corresponding plurality of transferrin receptors simultaneously, the avidity arising from such multivalent binding markedly exceeding the affinity of any single aptamer-receptor interaction and thereby promoting firm capture of the nanostructure at the brain endothelium and efficient triggering of transcytosis. Each aptamer is presented away from the dense surface of the cage by extension from a surface staple strand through a flexible poly-thymidine linker, the linker providing the conformational freedom and radial projection necessary for the aptamer to adopt its binding-competent fold and to reach its receptor without steric hindrance from the underlying nucleic acid lattice or from the stealth corona. The aptamer display is combined with the surface PEGylation described below, the two surface modifications being co-presented such that targeting and stealth functions operate together rather than in opposition.

### Transcytosis Mechanism and Brain Accumulation

The passage of the nanorobot from the systemic circulation into the brain parenchyma proceeds through an ordered sequence of events that may be described stepwise, the entire pathway being mediated by receptor-driven vesicular transport across the endothelial cell rather than by any breach of the barrier. In the first phase, following intravenous administration, the PEGylated nanorobot circulates within the bloodstream as a long-lived, stealth-coated particle, the surface PEG corona conferring a circulation half-life on the order of approximately six to twelve hours that affords the particle repeated and extended opportunity to perfuse the dense capillary network of the brain. In the second phase, as the circulating nanorobot transits the cerebral microvasculature, it is brought into proximity with the luminal surface of the brain microvascular endothelial cells, where the transferrin receptor is expressed at the elevated density characteristic of the brain endothelium.

In the third phase, the transferrin-receptor aptamers arrayed upon the exterior of the spherical cage engage the endothelial transferrin receptors through high-avidity multivalent binding, the simultaneous occupancy of multiple receptors anchoring the nanorobot firmly to the luminal membrane and clustering the engaged receptors. In the fourth phase, this engagement and clustering initiate receptor-mediated transcytosis, the membrane invaginating about the bound nanorobot to form an endocytic vesicle that internalizes the particle into the body of the endothelial cell. In the fifth phase, the resulting vesicle is trafficked vectorially across the interior of the endothelial cell from the luminal to the abluminal face along the transcytotic route, the nanorobot being conveyed within the membrane-bounded compartment in a manner that, consistent with the validated behavior of framework nucleic acid nanostructures, is substantially independent of lysosomal routing and therefore spares the cargo from degradative destruction. In the sixth and final phase, the vesicle fuses with the abluminal membrane and discharges the intact nanorobot into the brain parenchyma beyond the endothelial barrier, where it is positioned to act upon its target cells.

The efficiency of this pathway, expressed as the fraction of the administered dose that reaches the brain, is expected to fall within a range of approximately three to seven percent of the injected dose accumulating within the brain over a window of approximately six to twelve hours following administration, an expectation grounded in the brain-accumulation data reported for systemically administered DNA nanocages of comparable size and geometry. Although this fraction is a minority of the total administered dose, it represents an accumulation orders of magnitude greater than the fraction of large molecules that cross the intact barrier unaided, and it is sufficient to deliver a therapeutically meaningful quantity of genome-editing machinery to the brain, particularly given the catalytic and durable nature of genome editing, in which a finite quantity of editing complex installed within target cells can effect a lasting genetic correction.

A particular advantage of the platform architecture is that the efficiency of transcytosis is preserved notwithstanding the substantial mass of the editing cargo carried within the nanorobot. The genome-editing complexes housed by the platform, comprising large multidomain effector proteins together with their guide nucleic acids, collectively constitute a payload whose total molecular mass exceeds five hundred kilodaltons, a mass that would ordinarily be expected to impede or preclude transcytotic transport were it presented at the particle surface. The platform circumvents this difficulty through compartmentalization, in which the entire editing cargo is sequestered within the sealed internal compartments of the spherical cage and is thereby isolated from the exterior of the structure. Because the surface that the brain endothelium recognizes and transports is defined solely by the cage exterior, bearing its transferrin-receptor aptamers and its stealth corona, and because the massive editing cargo is hidden within the interior and does not present itself to the transport machinery, the transcytotic efficiency of the nanorobot is governed by the favorable size, geometry, and surface chemistry of the cage rather than by the prohibitive bulk of its contents. The large cargo is thus carried as protected internal freight across the barrier, and the efficiency of crossing is preserved despite a total cargo mass that would otherwise be incompatible with receptor-mediated transcytosis.

### PEGylation and Stealth Properties

The circulation behavior and stealth character of the nanorobot, upon which the foregoing crossing efficiency depends, are conferred by a defined surface modification with polyethylene glycol. The PEGylation is accomplished through the incorporation of staple strands bearing a cholesterol moiety conjugated to a polyethylene glycol chain, the polyethylene glycol being of a molecular weight of approximately two thousand or approximately five thousand daltons in preferred embodiments, designated respectively cholesterol-PEG-2000 and cholesterol-PEG-5000. These amphipathic staple conjugates are incorporated into the assembled structure at a surface density of approximately five to ten percent of the available surface staple positions, a proportion selected to provide effective stealth coverage while preserving sufficient surface real estate for the simultaneous display of the transferrin-receptor aptamers.

The mode of attachment exploits the amphipathic character of the cholesterol-PEG conjugate to anchor the stealth coating stably to the nucleic acid structure. The hydrophobic cholesterol moiety inserts into and anchors within the double-helical duplex regions of the DNA cage, partitioning into the lattice and securing the conjugate to the structure, while the hydrophilic polyethylene glycol chain extends outward from the surface into the surrounding aqueous medium. The collective projection of these outwardly extended polyethylene glycol chains forms a hydrated, brush-like stealth corona that envelops the exterior of the spherical cage and presents a sterically shielding, water-rich layer to the surrounding biological milieu.

The stealth corona confers several interrelated benefits that together extend the residence of the nanorobot in the circulation. By interposing a hydrated steric barrier between the nucleic acid surface and circulating biomolecules, the polyethylene glycol layer reduces the adsorption of serum proteins onto the particle and thereby suppresses the formation of the protein corona that would otherwise mark the particle for recognition. This reduction in protein adsorption in turn minimizes the nonspecific cellular uptake and the opsonin-mediated clearance by the mononuclear phagocyte system that rapidly remove uncoated nanostructures from the blood. The stealth corona additionally affords a measure of protection against nuclease attack upon the structural nucleic acid. The net effect of these benefits is the extension of circulation half-life to the approximately six-to-twelve-hour range noted above, a prolongation that is itself a direct determinant of brain delivery.

The role of the stealth coating in brain delivery is best understood as governing the fraction of the administered dose that survives in the circulation long enough to reach the cerebral vasculature and to engage the transferrin receptor. Brain accumulation is a kinetic competition between the slow, receptor-mediated extraction of nanorobots at the brain endothelium and the various clearance processes that remove nanorobots from the blood before they can be so extracted. By suppressing protein-corona formation, nonspecific uptake, and phagocytic clearance, and by thereby prolonging the circulatory lifetime of the particle, the stealth corona increases the number of passes that each nanorobot makes through the cerebral microvasculature and raises the cumulative probability that the particle is captured and transcytosed into the brain. The PEGylation and stealth function described in this subsection therefore operate in direct concert with the transferrin-receptor targeting and the validated cage geometry to yield the overall brain-delivery performance of the blood-brain barrier crossing subsystem, the stealth corona raising the fraction of dose that reaches the cerebral vasculature and the targeting array converting that surviving fraction into accumulated brain dose.

### Rationale for Sequential Release

The therapeutic payload of the present platform comprises three mechanistically distinct genome-and transcriptome-engineering enzymes housed within three separate internal compartments of the DNA origami nanorobot: a cytosine or adenine base editor in Compartment 1, a prime editor in Compartment 2, and a Cas13 effector complex in Compartment 3. A naive design would release all three editors into the target cell at the moment of internalization. Such simultaneous, undifferentiated release is disadvantageous for several interrelated reasons that the logic-gated sequential-release subsystem described in this section is engineered to overcome.

First, the three editors compete for shared cellular resources. Base editors and prime editors are large fusion proteins that, when delivered as messenger RNA or as ribonucleoprotein with guide RNA, draw upon the same pool of ribosomes for translation and upon overlapping chaperone and trafficking machinery for folding and intracellular distribution. When all three cargoes are presented to the cell at once, the finite translational and trafficking capacity is divided among them, so that no single editor achieves the local concentration required for efficient, complete conversion of its target locus before the cell mounts compensatory responses.

Second, and more critically, base editing and prime editing both act upon genomic DNA and both recruit endogenous DNA-repair machinery. Base editors that deaminate cytosine generate a uracil intermediate whose resolution depends on base-excision-repair handling, while prime editors install their edits through a 3′ flap that must be resolved by mismatch-repair and flap-endonuclease activity. When a base editor and a prime editor operate on the same cell, and potentially on nearby or overlapping loci, in the same time window, they place simultaneous and conflicting demands on these repair pathways. The repair intermediates of one editor can be mis-processed by the machinery recruited by the other, producing unintended deletions, insertions, or reversion of a desired edit.

Third, simultaneous release produces an unpredictable editing order. The biological outcome at a given locus frequently depends on which editor acts first; a base-edited sequence may alter the protospacer or primer-binding site that a prime editor relies upon, and vice versa. If the order in which the editors engage the genome is left to stochastic competition, the editing outcome varies from cell to cell, undermining both efficiency and reproducibility across the treated population.

Fourth, the Cas13 effector modulates RNA rather than DNA. If Cas13 is released concurrently with the DNA editors, it may degrade or alter the very transcripts encoding those editors, or modulate transcripts before the underlying DNA-level corrections have been written, defeating the intended hierarchy in which durable DNA-level changes are established before transient RNA-level modulation is layered on top.

The logic-gated sequential-release subsystem resolves all of these problems by imposing a defined temporal order on payload release: base editing is initiated and allowed to proceed before prime editing begins, and prime editing is initiated and allowed to proceed before Cas13 is released. By staggering release in this manner, each editor encounters a cellular environment in which it is not competing with the others for ribosomes, repair machinery, or substrate, so each can work toward completion before the next is introduced. That DNA nanodevices can be operated in a controlled, sequential, externally and internally triggered manner is established in the art of structural DNA nanotechnology, where logic-gated nanorobots release their payloads only upon satisfaction of defined molecular conditions [Douglas 2012], and where cascaded sense-and-release operations have been demonstrated in which the output of one logic operation serves as an input to a subsequent operation [Wang 2021]. The present subsystem builds on these established principles to coordinate three editors in strict succession.

### Gate 1—Dual-Aptamer and Gate Controlling the Base-Editor Compartment

Gate 1 governs the lid of Compartment 1, which contains the base editor, and implements the Boolean function (Disease Biomarker) AND (Cell-Type Marker). The compartment is built as a hollow DNA origami vessel in the tradition of scaffolded DNA origami, in which a long single-stranded scaffold is folded by short staple strands into a defined three-dimensional shape with an addressable interior [Rothemund 2006 2009]. The lid of this vessel is held in the closed configuration by two independent blocking strands, designated Block A and Block B, each of which clasps the lid to the body of the compartment through duplex hybridization. So long as either blocking strand remains engaged, the lid cannot open and the base editor remains sequestered.

Each blocking strand is released by a distinct molecular recognition event. Block A is removed when a disease-biomarker aptamer, integrated into the gate, binds its cognate disease-associated target; the binding event drives a conformational rearrangement of the aptamer that liberates the toehold on Block A. Block B is removed in the analogous fashion when a cell-type-marker aptamer binds its cognate cell-surface or intracellular marker characteristic of the intended target cell type. The lid opens only when both Block A and Block B have been released, which occurs only when both the disease biomarker and the cell-type marker are simultaneously present; thus the gate computes a true AND, and the base editor is delivered exclusively to cells that are both diseased and of the correct lineage.

The mechanism by which each aptamer-binding event removes its blocking strand is toehold-mediated strand displacement. Each blocking duplex carries a single-stranded overhang, the toehold, of between six and twelve nucleotides. Upon target binding, the aptamer exposes or repositions a displacing domain whose sequence is complementary to the toehold and to the adjacent duplex region. The displacing strand nucleates at the exposed toehold and then propagates through a branch-migration process that progressively peels the blocking strand away from the lid, releasing it. A toehold length in the six-to-twelve nucleotide range is chosen to make the displacement reaction both thermodynamically favorable and kinetically prompt while avoiding spontaneous, leaky opening in the absence of the cognate trigger. This dual-aptamer AND-gate architecture, in which two independent binding events are each required to unlatch a mechanical DNA structure before a payload is exposed, follows the logic-gated DNA nanorobot design demonstrated in the art [Douglas 2012].

The two aptamers are selected for high affinity and specificity. The disease-biomarker aptamer is chosen to bind its target with a dissociation constant in the approximate range of twenty to one hundred nanomolar, so that it reliably samples and reports the presence of the disease biomarker at physiologically relevant concentrations without being so tight that it binds transiently elevated background. The cell-type-marker aptamer is chosen to recognize a marker that distinguishes the intended target cell population, and is likewise selected for an affinity that ensures robust recognition within the target cell while minimizing engagement in off-target cells.

When both aptamers are engaged and both blocking strands have been displaced, the lid of Compartment 1 swings clear of the vessel mouth, creating a pore in the wall of the nanorobot on the order of eight to fifteen nanometers across. This aperture is sized to permit the base editor, whether delivered as a folded ribonucleoprotein or as an associated cargo complex, to diffuse out of the compartment and into the surrounding cytoplasm. Egress is not instantaneous; the base editor diffuses out of the opened compartment over a period of approximately thirty to sixty minutes, producing a graded rather than abrupt local rise in editor concentration that favors orderly engagement of the target locus.

In terms of timing, Gate 1 is the first gate in the cascade and is the gate that responds to disease detection following cell entry. It activates upon co-detection of the disease biomarker and the cell-type marker after the nanorobot has entered the target cell, and is configured to open on the order of six to eight hours after cell entry. The opening of Gate 1 establishes the temporal reference point from which the two downstream gates are timed, and it produces the molecular confirmation signal upon which Gate 2 is made contingent, as described below.

### Gate 2—Ph-Sensitive i-Motif and Gate Controlling the Prime-Editor Compartment

Gate 2 governs the lid of Compartment 2, which contains the prime editor, and implements the Boolean function (Gate 1 confirmation) AND (intracellular pH below approximately 6.5). The first input to this gate is a confirmation signal generated by the opening of Gate 1, and the second input is an acidic local pH. Only when both conditions are met does Gate 2 open, ensuring both that the prime editor is released only after base editing has been licensed to proceed and that release occurs in the appropriate subcellular environment. More generally, the pH-sensing threshold of the second gate is selected to lie below approximately 7.0, and in representative embodiments below approximately 6.5, so that the gate remains closed at the near-neutral pH of blood and interstitial fluid (approximately 7.4) and opens only upon encountering the mildly acidic intracellular and endosomal environment that follows internalization.

The pH responsiveness of Gate 2 derives from cytosine-rich i-motif sequences incorporated into the lid. The i-motif is a four-stranded DNA secondary structure in which two parallel-stranded duplexes are held together in an antiparallel, intercalated arrangement by hemiprotonated cytosine-cytosine base pairs; this tetrameric folded structure forms under mildly acidic conditions and unfolds to a random coil at neutral pH [Gehring 1993]. At the physiological extracellular and cytosolic pH of approximately 7.4, the cytosine-rich segments of the Gate 2 lid remain unfolded as a random coil, and the lid retains the conformation that keeps Compartment 2 closed. A representative pH-responsive i-motif DNA element employed in the lid comprises consecutive cytosine-rich tracts separated by short intervening loops, in which the runs of cytosine provide the cytosine-cytosine pairing required for folding and the intervening residues provide the connecting loops.

The first input, the Gate 1 confirmation, is supplied through a confirmation-lock mechanism. When Gate 1 opens, it releases a confirmation-signal strand that had been held in reserve. This confirmation-signal strand is required to be present for the Gate 2 lid to be able to respond to pH at all; in its absence, the i-motif segments are held in a configuration that cannot productively fold and destabilize the lid even if the local pH falls. The confirmation-signal strand hybridizes to a complementary domain on the Gate 2 lid and thereby unlocks the pH-sensing capacity of the i-motif, arming the gate. This interlock prevents premature opening of Compartment 2: even if the nanorobot were to encounter an acidic microenvironment before Gate 1 had fired, the prime editor would not be released, because the pH input alone is insufficient to satisfy the AND condition.

The second input, acidic pH, is supplied naturally by the cell's own internalization machinery. Following receptor-mediated uptake, the nanorobot is trafficked through the endosomal system, where the luminal pH falls progressively from the near-neutral value of approximately 7.4 characteristic of the early compartment, through an intermediate value of approximately 6.5, and onward toward approximately 5.5 as the compartment matures. This progressive acidification provides a reliable, endogenous chemical timer. When the confirmation-signal strand is present and the local pH has fallen below approximately 6.5, the cytosine-rich segments fold into the i-motif structure. The folding of the i-motif shortens and rigidifies those segments, exerting a mechanical strain that destabilizes the lid of Compartment 2 and unlatches it, releasing the prime editor.

Because Gate 2 requires the conjunction of the Gate 1 confirmation strand and an acidic pH, it performs a cascaded AND-AND operation in which the output of the first logic gate is consumed as an input to the second, a mode of cascaded sense-and-release operation established for DNA logic-gated nanodevices in the art [Wang 2021]. In terms of timing, Gate 2 is configured to open approximately four to six hours after Gate 1. This interval ensures that base editing has begun, and ideally has substantially progressed, before the prime editor is introduced into the cytoplasm, so that the two DNA-level editors do not compete for repair machinery or substrate within the same window.

### Gate 3—NLS/Importin and Gate Controlling the Cas13 Compartment

Gate 3 governs the lid of Compartment 3, which contains the Cas13 effector complex, and implements the Boolean function (Gate 2 confirmation) AND (nuclear-localization recognition). The first input is a second confirmation signal generated by the opening of Gate 2, and the second input is recognition of an exposed nuclear localization signal by the cellular nuclear-import machinery. Only when both conditions are satisfied does Gate 3 open, so that the Cas13 effector is released only after prime editing has been licensed to proceed and only in proximity to the nucleus.

The lid of Compartment 3 bears a representative nuclear localization signal peptide comprising a canonical SV40 large T-antigen nuclear localization signal, or alternatively a bipartite nuclear localization signal comprising two clusters of basic residues separated by a short spacer [Kalderon 1984]. In the resting state, this nuclear localization signal is not available to the cellular import machinery: it is masked by a hybridized DNA blocking strand that covers the signal and prevents its recognition. So long as the blocking strand remains in place, the nuclear-import apparatus cannot engage the lid, and Compartment 3 stays closed regardless of subcellular location.

The first input, the Gate 2 confirmation, removes this mask. When Gate 2 opens, it releases a second confirmation-signal strand. This strand is complementary to the blocking strand that conceals the nuclear localization signal, and through toehold-mediated strand displacement it removes the blocking strand, exposing the SV40 nuclear localization signal on the surface of the Compartment 3 lid. Thus the nuclear localization signal becomes available for recognition only after, and as a direct consequence of, the firing of Gate 2; this is the first half of the AND condition.

The second input is supplied by the cellular nuclear-import machinery itself. Once the nuclear localization signal is exposed, importin-beta, acting directly or together with an importin-alpha adaptor, recognizes and binds the exposed basic-residue signal. This binding event, occurring as the nanorobot is trafficked toward and docks at the nuclear envelope, applies a recognition-coupled mechanical force to the lid. The engagement of importin with the nuclear localization signal induces a conformational change in the lid that opens Compartment 3 and releases the Cas13 effector complex near to, or within, the nucleus, where it is positioned to act upon nascent and resident transcripts. The use of nuclear-import recognition as the terminal physical trigger both gates the release on subcellular position and harnesses an endogenous targeting pathway to co-localize the released Cas13 with its RNA substrates.

In terms of timing, Gate 3 is configured to open approximately eight to twelve hours after Gate 2. This comparatively long interval is deliberate: it allows base editing and prime editing, both of which write durable changes at the DNA level, to lock in and resolve before RNA-level modulation by Cas13 begins. By the time Cas13 is released, the genomic corrections established by the upstream editors are committed, so the transient transcriptome-level activity of Cas13 is layered on top of a stabilized genomic foundation rather than competing with ongoing DNA editing. The operation of three contingent logic stages in strict succession, with each stage validated by the molecular output of the preceding stage, follows the three-tier cascaded sense-and-release validation established for DNA logic-gated nanodevices in the art [Wang 2021].

### Overall Temporal Coordination

The three gates together impose a single, strictly ordered temporal program on the platform, which can be narrated as a continuous timeline from administration through sustained effect. The intervals given are nominal and reflect the design targets of the gating chemistry described above; they coordinate the four payload activities so that each acts in its own window.

At time zero, the nanorobot population is administered intravenously and enters the systemic circulation. Over the following approximately two to four hours, the nanorobots cross the blood-brain barrier by transferrin-receptor-mediated transcytosis, a process driven by the targeting ligands borne on the exterior of the device, accumulating in the target tissue. Over approximately the next interval, spanning roughly four to six hours after administration, the nanorobots are internalized into target cells by receptor-mediated uptake and begin their passage through the endosomal system, where the progressive acidification that will later supply the pH input to Gate 2 commences.

At approximately six to eight hours after administration, Gate 1 opens. Co-detection of the disease biomarker and the cell-type marker satisfies the first AND condition, both blocking strands are displaced, the eight-to-fifteen-nanometer pore forms, and the base editor diffuses out over the ensuing thirty to sixty minutes to begin writing cytosine or adenine conversions at the target locus. The opening of Gate 1 simultaneously releases the confirmation-signal strand that arms Gate 2.

At approximately ten to fourteen hours after administration, Gate 2 opens. By this time the Gate 1 confirmation strand is present and the endosomal pH has fallen below approximately 6.5; the cytosine-rich i-motif segments fold, the lid of Compartment 2 is destabilized, and the prime editor is released. Because Gate 1 fired several hours earlier, base editing is already under way when the prime editor appears, so the two DNA-level editors do not contend for repair machinery or substrate in the same instant. The opening of Gate 2 releases the second confirmation-signal strand that unmasks the nuclear localization signal of Gate 3.

At approximately eighteen to twenty-six hours after administration, Gate 3 opens. The exposed SV40 large T-antigen nuclear localization signal is recognized by importin, the lid of Compartment 3 undergoes its conformational change, and the Cas13 effector is released near or within the nucleus. By this stage the genomic edits installed by the base editor and the prime editor have had time to lock in, so the RNA-level modulation performed by Cas13 builds upon a settled genomic substrate rather than interfering with active DNA editing.

Over the subsequent interval, spanning approximately two to seven days, the combined activities of the three editors produce a sustained therapeutic effect: the DNA-level corrections persist as durable changes to the genome, while the Cas13 activity provides ongoing transcript-level modulation during its functional lifetime. Because the Cas13 activity is transient relative to the permanent DNA edits, an optional repeat administration may be scheduled at approximately two to four weeks to maintain the Cas13-mediated component of the therapy without re-editing the already-corrected genome.

This temporal coordination is not achievable with conventional delivery methods that release all therapeutic components into the cell simultaneously. In a simultaneous-release scheme there is no mechanism to ensure that base editing precedes prime editing or that DNA-level editing precedes RNA-level modulation; the order of engagement is left to stochastic competition, with the attendant losses in efficiency, reproducibility, and safety described at the outset of this section. In the present subsystem, by contrast, each successive gate is made contingent on the completion of the prior gate: Gate 2 cannot fire without the Gate 1 confirmation strand, and Gate 3 cannot fire without the Gate 2 confirmation strand. Because each confirmation strand is generated only when its upstream gate has actually opened, the cascade is strictly ordered, self-verifying, and incapable of running out of sequence, and the platform thereby delivers a defined, repeatable program of base editing, then prime editing, then Cas13 modulation within each treated cell.

### Disease Overview

Huntington's disease is an autosomal dominant neurodegenerative disorder caused by a single mutation in the HTT gene located on the short arm of chromosome 4 at locus 4p16.3. The mutation consists of an unstable expansion of a cytosine-adenine-guanine (CAG) trinucleotide repeat situated within exon 1 of the gene. In unaffected individuals, this repeat tract spans approximately 10 to 35 units. When the repeat number expands to 40 or more, the disease becomes fully penetrant, meaning that essentially every carrier who lives a normal lifespan will eventually develop the clinical syndrome. The intermediate range between these boundaries is associated with incomplete penetrance and meiotic instability that can produce affected offspring. Because a single expanded allele is sufficient to cause disease, each child of an affected parent carries a fifty percent probability of inheriting the pathogenic expansion.

At the molecular level, the expanded CAG tract is translated into an abnormally long polyglutamine stretch near the amino terminus of the huntingtin protein. This expanded polyglutamine tract confers a toxic gain of function upon the mutant huntingtin protein, promoting protein misfolding, the formation of intracellular aggregates and nuclear inclusions, transcriptional dysregulation, mitochondrial dysfunction, impaired axonal transport, and disrupted proteostasis. The toxicity is graded with respect to repeat length, such that longer expansions correlate with earlier onset and more aggressive progression. A particularly important contributor to disease biology is somatic repeat instability, whereby the CAG tract continues to expand within vulnerable post-mitotic neurons over the lifetime of the individual, driving a self-amplifying cycle of toxicity that is thought to underlie the selective regional vulnerability of the disorder.

The principal site of neurodegeneration is the striatum, where the GABAergic medium spiny neurons that express the marker DARPP-32 (dopamine-and cyclic-AMP-regulated phosphoprotein of molecular weight thirty-two kilodaltons) are preferentially lost. The loss of these striatal projection neurons disrupts the basal ganglia circuitry that governs movement and contributes to the characteristic chorea, dystonia, and impaired voluntary motor control. As the disease advances, neurodegeneration extends to the cerebral cortex and other regions, producing the progressive triad of motor dysfunction, cognitive decline, and psychiatric disturbance. Huntington's disease is uniformly fatal, typically over a span of fifteen to twenty years from the onset of overt symptoms, and there is at present no curative or disease-modifying therapy available.

Existing therapy is purely symptomatic and does nothing to alter the underlying genetic lesion or the relentless course of neurodegeneration. The vesicular monoamine transporter inhibitor tetrabenazine and its derivatives are used to suppress chorea but carry meaningful neuropsychiatric liabilities, do not protect neurons, and offer no benefit for the cognitive or functional decline that ultimately determines disability. The most advanced experimental strategies have encountered fundamental obstacles. A widely studied antisense oligonucleotide program directed against huntingtin messenger RNA, the antisense oligonucleotide tominersen, advanced into late-stage clinical evaluation but was halted after the higher-dose regimens were associated with dose-dependent clinical worsening rather than benefit, illustrating the danger of unselective and non-titratable suppression of huntingtin. A surgically delivered gene therapy program employing an adeno-associated viral vector, the AAV program AMT-130, requires direct intracranial neurosurgical administration by stereotactic intrastriatal injection; the diffusion of vector from the injection tracks is limited, the dose cannot be adjusted after administration, and the irreversibility and invasiveness of the procedure constrain its applicability. Taken together, these experiences establish a clear clinical need for an approach that is noninvasive in its delivery, that acts at multiple complementary molecular levels rather than relying on a single mechanism, and that is precisely titratable so that the degree and timing of intervention can be matched to the individual disease state.

### Why Three Modalities Are Necessary

The central rationale for the tri-modal platform described herein is that no single gene-editing or gene-silencing modality, acting alone, is capable of reducing toxic mutant huntingtin to a degree sufficient to arrest disease while simultaneously preserving the essential physiological functions of normal huntingtin. Each available modality addresses a different facet of the disease and carries a different ceiling of achievable effect, and only their coordinated combination overcomes the limitations inherent in any one of them.

Base editing, when applied alone to the expanded CAG tract, is configured to interrupt the purity of the repeat by converting individual CAG codons to alternative codons that do not encode glutamine or that break the contiguous run. In the configurations contemplated here, this interruption strategy is expected to alter only approximately forty to sixty percent of the targetable CAG repeats within the tract. While such interruption meaningfully reduces polyglutamine toxicity and is expected to suppress further somatic expansion, a substantial fraction of the toxic protein remains because the tract is only partially disrupted and the residual uninterrupted repeats continue to be translated into an expanded polyglutamine stretch. Base editing alone therefore leaves a considerable burden of toxic protein in place.

Prime editing, when applied alone, is configured to excise a defined block of CAG repeats from the tract so as to shorten the expansion. In the configurations contemplated here, prime editing alone is expected to delete only approximately thirty to fifty percent of the repeats. Although shortening the tract toward or below the pathogenic threshold is a powerful intervention, the efficiency of programmable deletion at a long, structurally challenging repeat is inherently limited, so a single modality of this kind cannot be relied upon to drop every targeted allele below threshold across the neuronal population.

Cas13-mediated knockdown of huntingtin messenger RNA, when applied alone, is expected to reduce target transcript levels by approximately fifty to seventy percent. This approach acts rapidly and is reversible, but it operates entirely at the post-transcriptional level and does nothing to correct the underlying genetic defect. Because the messenger RNA is continuously regenerated from the unaltered mutant allele, durable benefit would require lifelong, repeated dosing, with all the attendant risks of cumulative exposure and the demonstrated danger, seen in the halted antisense oligonucleotide experience, of non-titratable chronic suppression.

When these three modalities are combined in a coordinated sequence, their individual effects compound multiplicatively rather than merely adding, because each modality acts upon the fraction of toxic species left uncorrected by the preceding one. The platform is configured so that base editing first interrupts a portion of the repeats, prime editing then deletes a portion of what remains, and Cas13 finally degrades a portion of the messenger RNA still being produced, yielding a combined reduction in toxic mutant huntingtin exceeding ninety percent. Such a depth of correction is not attainable by any single modality and is the defining advantage of the tri-modal design.

A further and critical consideration drives the choice of a partial, titratable, multi-level strategy rather than a wholesale knockout. The huntingtin gene is essential; its protein product participates in neuronal development, vesicular and axonal transport, transcriptional regulation, and the maintenance of neuronal survival, and complete elimination of huntingtin is undesirable and potentially harmful. The platform is therefore deliberately designed to lower the toxic mutant species below the threshold of pathogenicity while retaining adequate functional huntingtin, an objective that demands the precision and graded control afforded by combining base editing, prime editing, and messenger RNA knockdown under independent release control.

### Compartment 1—Base Editing for CAG Interruption

The first compartment of the nanorobot houses an adenine base editor of the ABE8e class together with its programmed guide RNA. The ABE8e editor [Richter 2020] is selected for its high catalytic activity and rapid deamination kinetics, properties that favor efficient editing within the limited residence time of the cargo at the genomic target. This compartment is configured to be released first in the therapeutic sequence, ensuring that base-editing-mediated interruption of the repeat tract is the initial intervention applied to the mutant allele.

The therapeutic rationale of this compartment is to interrupt the contiguous purity of the expanded CAG repeat. A pure, uninterrupted run of CAG codons is both the template for an unbroken polyglutamine tract and the substrate most prone to somatic expansion. By introducing base changes that convert selected CAG codons into codons that do not encode glutamine or that otherwise break the repeat periodicity, the platform reduces polyglutamine toxicity and is expected to stabilize the tract against further somatic growth. Representative interruption strategies include a CAG-to-CGG alteration, in which an adenine base editor of the ABE8e class converts the central adenine of selected CAG codons (an adenine-to-guanine edit), thereby altering the encoded residue and directly breaking the polyglutamine-encoding run; and a synonymous CAG-to-CAA interruption, in which a cytosine base editor of the CBE4max or AncBE4max class acts on the antisense strand (converting the cytosine of the complementary CTG triplet to thymine, so that the sense codon reads CAA), preserving the glutamine reading at the protein level while disrupting the nucleotide-level repeat that drives instability. The capacity of base editing to act constructively upon the HTT CAG tract in this manner is supported by evidence that base editing can interrupt the CAG repeat in huntingtin [Choi 2024] and that base editing of trinucleotide repeats reduces somatic expansion [Matuszek 2025].

In a representative configuration, the guide RNA directs the ABE8e editor to the CAG-rich region of HTT exon 1. A representative single guide RNA spacer targeting CAG repeat regions of the HTT gene is identical in sequence to the protospacer on the CAG-containing non-target strand of the repeat and accordingly directs the guide RNA to hybridize with the complementary CTG-containing target strand. Upon R-loop formation, the displaced non-target strand presents the repeat-resident adenines on which the adenine deaminase of the ABE8e editor acts. A canonical SpCas9-derived editor, however, requires an NGG protospacer-adjacent motif, and a pure, uninterrupted CAG tract contains no such motif on either strand: the two strands of the repeat read CAGCAG and CTGCTG, neither of which contains the requisite GG dinucleotide. A canonical SpCas9-based editor must therefore rely on an NGG motif located in the unique sequence immediately flanking one end of the repeat, which fixes a single targeting register at that boundary. To instead distribute interruption events across multiple registers along the tandem tract, the editor is configured with a protospacer-adjacent-motif-flexible Cas9 nickase architecture, such as an SpCas9-NG variant that recognizes relaxed NG motifs or a near-protospacer-adjacent-motif-independent SpRY variant, which permits the deaminase editing window to be positioned at successive registers along the repeat. The expected outcome of this compartment, acting alone, is interruption of approximately forty to sixty percent of the targetable CAG repeats.

The release of the Compartment 1 cargo is governed by a logic-controlled trigger designated Gate 1, which is configured as the Boolean conjunction (mutant huntingtin) AND (DARPP-32). The first input confirms the presence of the disease-causing expanded huntingtin species, and the second input confirms that the cargo resides within a DARPP-32-positive striatal medium spiny neuron, the cell type most relevant to Huntington's disease pathology. Only when both conditions are satisfied does Gate 1 permit release of the base editor, restricting the most aggressive and earliest intervention to the precise pathological context for which it is intended and sparing non-target cells.

### Editor Selection and Targeting Logic

The selection of an adenine base editor of the ABE8e class reflects a balance between editing efficiency and the constraints of the nanorobotic delivery context. The high turnover of this editor compensates for the brief window during which the released cargo is catalytically available, and its compatibility with both the standard NGG protospacer-adjacent motif and protospacer-adjacent-motif-flexible Cas9 architectures simplifies the identification of targetable positions adjacent to and within the repeat. Confining the action of this compartment to genomic interruption of the repeat, rather than to transcript-level effects, situates the first intervention at the most upstream point in the disease cascade, where a durable, heritable correction of the somatic genome can be achieved within the targeted neurons.

### Compartment 2—Prime Editing for CAG Repeat Deletion

The second compartment houses a prime editing system of the PE5 configuration, comprising an engineered reverse-transcriptase-fused nickase paired with a prime editing guide RNA and the accessory components that define the PE5 and PEmax architecture [Anzalone 2019][Chen 2021]. This compartment is configured to be released second in the therapeutic sequence, acting upon the repeat tract after base-editing interruption has been applied by the first compartment.

The therapeutic rationale of this compartment is to physically shorten the expanded CAG tract by programmed deletion, lowering the repeat number toward or below the pathogenic threshold. Whereas base editing alters the sequence content of the repeat without changing its length, prime editing is uniquely suited to removing a defined block of repeats and thereby converting a fully penetrant expanded allele into a tract within or near the benign range. In a representative configuration, the system is designed to delete approximately ten to twenty CAG repeats, for example reducing an allele of forty-five repeats, written as (CAG)45, to approximately twenty-five repeats, written as (CAG)25, a length below the threshold of full penetrance.

The prime editing guide RNA is designed to target the CAG-rich region of HTT exon 1, with the editing template and the flanking primer-binding and reverse-transcription template sequences configured so as to bridge across and delete a central block of repeats while preserving the surrounding exon 1 sequence in frame. The PE 5 configuration is selected for its enhanced editing efficiency and its incorporation of mechanisms that improve the precision and yield of programmed insertions and deletions at challenging loci such as the structured CAG repeat. The expected outcome of this compartment, acting alone upon the residual repeats not already addressed by base editing, is deletion of approximately thirty to fifty percent of the targeted repeats.

The release of the Compartment 2 cargo is governed by a logic-controlled trigger designated Gate 2, configured as the Boolean conjunction (Gate 1 complete) AND (pH below 6.5). The first input enforces strict sequencing, ensuring that prime editing is initiated only after the base-editing step of Compartment 1 has been carried out, so that the two genome-modifying activities act in their intended order rather than competing for the same substrate simultaneously. The second input, a local pH below 6.5, confirms the appropriate subcellular microenvironment for cargo release, providing an additional layer of spatial control that gates deployment of the prime editor to the intended intracellular location.

### Compartment 3—Cas13 mRNA Knockdown

The third compartment houses a Cas13d effector of the CasRx class together with a multiplexed guide RNA array [Konermann 2018]. This RNA-targeting nuclease operates at the post-transcriptional level, recognizing and cleaving huntingtin messenger RNA without altering the genome. This compartment is configured to be released third and last in the therapeutic sequence, providing an immediate reduction of any toxic transcript that continues to be produced after the two genome-level interventions have been applied.

The therapeutic rationale of this compartment is to suppress the pool of mutant huntingtin messenger RNA that remains after base editing and prime editing, thereby lowering the level of newly synthesized toxic protein during the period in which the genomic corrections take effect and across any alleles or cells incompletely corrected by the upstream modalities. To achieve robust and durable knockdown, the compartment employs a multiplexed array of CRISPR RNAs directed against several distinct sites along the huntingtin transcript, so that knockdown does not depend upon the accessibility of any single site and is buffered against local secondary structure or sequence variation.

In a representative configuration the array comprises three multiplexed CRISPR RNA spacers targeting huntingtin messenger RNA. The first, designated crRNA-1, is targeted to a site in exon 1 upstream of the CAG region. The second, designated crRNA-2, is targeted to a site in exon 10 of the huntingtin transcript. The third, designated crRNA-3, is targeted to a site within the three-prime untranslated region of the huntingtin transcript. By engaging the transcript at an upstream coding position, a central coding position, and the three-prime untranslated region simultaneously, the array achieves redundant and additive cleavage along the length of the messenger RNA. The expected outcome of this compartment, acting through the combined activity of the multiplexed array, is greater than eighty-five percent knockdown of the targeted transcript. The capacity of Cas13d and CasRx systems to lower huntingtin and to ameliorate disease phenotypes in models of Huntington's disease is supported by evidence that such RNA-targeting effectors alleviate Huntington's disease phenotypes [Morelli 2023] and relieve Huntington's disease symptoms [Lin 2025].

The release of the Compartment 3 cargo is governed by a logic-controlled trigger designated Gate 3, configured as the Boolean conjunction (Gate 2 complete) AND (nuclear proximity). The first input enforces sequential deployment, ensuring that messenger RNA knockdown begins only after the prime-editing step of Compartment 2 has been completed, so that all three modalities act in their designed order. The second input, a sensed condition of nuclear proximity, positions the release of the Cas13d effector where nascent and recently exported huntingtin transcript is most concentrated, maximizing the efficiency of transcript engagement at the moment of deployment.

### Combined Therapeutic Outcome

The defining advantage of the platform emerges when the three compartments act in their coordinated sequence, because their effects compound multiplicatively upon a shrinking residual pool of toxic mutant huntingtin. Considered as a worked example using representative central values, base editing in the first stage corrects approximately fifty percent of the toxic species, leaving approximately fifty percent. Prime editing in the second stage then corrects approximately forty percent of that remainder, removing approximately twenty percent of the original burden and leaving approximately thirty percent. Cas13-mediated knockdown in the third stage then reduces approximately seventy percent of what still remains, removing approximately twenty-one percent of the original burden and leaving approximately nine percent. The net result is a reduction in toxic mutant huntingtin of greater than ninety percent. This depth of correction is achievable only by combining all three modalities in a coordinated sequence; no single modality, constrained by the individual ceilings described above, can approach it, and the sequencing enforced by the cascaded release gates is what allows each modality to act efficiently upon the fraction the preceding one could not reach.

The expected clinical outcomes of achieving such a reduction are framed as the configured and anticipated consequences of lowering toxic mutant huntingtin below the threshold of ongoing pathology while preserving essential huntingtin function. The platform is configured to be capable of arresting the progressive atrophy of the striatum by removing the toxic driver of medium spiny neuron loss, to produce improvement on motor and functional rating scales as the basal ganglia circuitry is relieved of toxic burden, and to stabilize or improve cognition as cortical and subcortical networks are protected from continued degeneration. Because the genome-level corrections delivered by the first two compartments are durable, and because the platform also acts to suppress somatic expansion of the repeat, the anticipated benefit is configured to be sustained rather than transient, in contrast to approaches that depend on continuous re-dosing.

The platform is configured for use across the spectrum of disease states. In presymptomatic carriers, defined by a CAG repeat of forty or more in the absence of overt clinical signs, the platform is intended for disease prevention, lowering the toxic species and shortening the tract before neurodegeneration becomes established. In early symptomatic patients, the platform is intended to halt further progression and to permit partial reversal of dysfunction in neurons that remain viable. In patients with advanced disease, the platform is intended to slow further progression and to improve quality of life by relieving the toxic burden upon surviving neurons.

This coordinated, noninvasive, and titratable strategy stands in direct contrast to the single-modality and invasive approaches that have foundered. Where the halted antisense oligonucleotide program demonstrated that non-titratable chronic suppression of huntingtin can produce dose-dependent worsening, the present platform applies graded, multi-level intervention under independent release control, lowering toxic huntingtin while sparing the essential functions of the normal protein. Where the surgically delivered adeno-associated viral approach requires irreversible intracranial injection with limited diffusion and no capacity for post-administration adjustment, the present platform is delivered noninvasively across the blood-brain barrier, distributes through the affected tissue, restricts its most aggressive activity to DARPP-32-positive striatal neurons bearing mutant huntingtin, and permits the depth and timing of intervention to be matched to the individual disease state through its cascaded, logic-gated release architecture.

### Additional Disease Embodiments

The three-compartment nanorobot described in the foregoing sections is a modular therapeutic chassis whose disease specificity is conferred almost entirely by the nucleic-acid programs it carries rather than by its physical architecture. In each of the embodiments set forth below, the spherical DNA origami chassis, the blood-brain-barrier-crossing surface chemistry, and the three-tier sequential gating logic that governs payload release remain unchanged. What changes from one indication to the next is the loaded guide-sequence set: the single guide RNAs that direct the Compartment 1 base editor, the prime editing guide RNAs (pegRNAs) that direct the Compartment 2 prime editor, and the CRISPR RNAs (crRNAs) that direct the Compartment 3 Cas13 effector, together with, where useful, the biomarker-responsive or cell-type-selective aptamers presented at the second gating tier. Because reconfiguration is accomplished by substituting these short, chemically synthesized oligonucleotides into pre-validated cargo modules, a single manufacturing and quality framework supports an open-ended family of indications, and the molecular nature of each genetic lesion dictates which of the three editing modalities is assigned to address it.

The same allocation principle governs every embodiment that follows. A point mutation that must be reverted to wild type, or installed as a protective allele, is generally addressed by the cytosine or adenine base editor in Compartment 1 or the prime editor in Compartment 2, depending on whether the required change lies within the accessible transition window or requires a transversion, small insertion, or precise templated rewrite. A pathological short-tandem-repeat expansion is addressed by the prime editor configured for repeat contraction and, in parallel, by the Cas13 effector configured to degrade the toxic repeat-containing transcript. A dosage imbalance or a dominant gain-of-function transcript is addressed at the RNA level by Cas13 knockdown. The descriptions below specify, for each disease, the causative gene or locus, the molecular pathology, the assigned tri-modal strategy, and the therapeutic effect expected when the nanorobot is so configured.

### Familial Alzheimer's Disease

Autosomal dominant familial Alzheimer's disease is caused by pathogenic mutations in the amyloid precursor protein gene (APP) on chromosome 21 and in the presenilin genes PSEN 1 and PSEN2, which encode the catalytic subunit of the gamma-secretase complex. Representative lesions include the APP Swedish double mutation KM670/671NL, which increases beta-secretase cleavage and total amyloid-beta output; the APP London variant V717I, which shifts gamma-secretase processing toward the longer, more aggregation-prone amyloid-beta 42 species; and presenilin missense mutations such as PSEN1 M146L and PSEN1 A246E, which alter the gamma-secretase cleavage register. Each of these is a defined single-nucleotide or near-single-nucleotide change, making them well suited to precise correction.

In the configured nanorobot, the Compartment 1 base editor is programmed with single guide RNAs that position the target codon within the editing window so that an adenine or cytosine deaminase reverts the pathogenic substitution to the wild-type sequence [Gaudelli 2017][Komor 2016]; the choice between adenine and cytosine base editing follows the identity of the required transition, and where the lesion lies outside a clean transition window the Compartment 2 prime editor is substituted instead [Anzalone 2019]. The same prime editor is used affirmatively to install the APP A673T Icelandic protective variant, a substitution adjacent to the beta-secretase cleavage site that reduces amyloidogenic processing; because A673T requires a precise templated change at a defined position, a pegRNA encoding the protective allele directs its installation [Anzalone 2019].

The Compartment 3 Cas13 effector provides a complementary, transcript-level arm. Programmed with crRNAs against MAPT messenger RNA, it lowers tau protein available for hyperphosphorylation and aggregation; programmed instead against BACE1 messenger RNA, it reduces beta-secretase activity upstream of amyloid-beta generation [Abudayyeh 2017]. The expected configured effect is a coordinated reduction in amyloid-beta production, a shift of any residual amyloid-beta toward shorter, less aggregation-prone species, and an attenuation of downstream tau pathology, addressing both arms of the amyloid-tau cascade from a single dose.

### LRRK2-Associated Parkinson's Disease

The most common monogenic cause of Parkinson's disease is the G2019S mutation in the leucine-rich repeat kinase 2 gene (LRRK2), a gain-of-function substitution within the kinase domain that elevates LRRK2 catalytic activity and drives downstream phosphorylation of Rab GTPases, lysosomal dysfunction, and neuronal loss. Because G2019S is a single base substitution producing a hyperactive enzyme, it is addressed primarily by precise reversion rather than by knockdown of the whole gene, preserving physiological LRRK2 function.

The Compartment 1 base editor is programmed with a single guide RNA that places the G2019S codon in the deaminase window so that the activating substitution is reverted to the wild-type residue, normalizing kinase activity at the genomic level [Gaudelli 2017][Komor 2016]. Where the surrounding sequence context disfavors clean base editing, the Compartment 2 prime editor is configured with a pegRNA encoding the wild-type codon as an alternative precise route [Anzalone 2019]. Because synuclein aggregation is a convergent feature of LRRK2 disease, the Compartment 3 Cas13 effector is programmed with crRNAs against alpha-synuclein (SNCA) messenger RNA to lower the cellular pool of aggregation-prone alpha-synuclein in parallel [Abudayyeh 2017].

To bias delivery toward the vulnerable cell population, the second gating tier may present a dopaminergic-neuron-selective aptamer, for example an aptamer recognizing a surface determinant such as NCAM1, so that payload release is favored in the substantia nigra dopaminergic neurons most affected by the disease. The expected configured effect is a reduction in pathogenic LRRK2 kinase activity through genomic correction together with a lowered burden of alpha-synuclein aggregates, slowing the convergent neurodegenerative process.

### Amyotrophic Lateral Sclerosis

Amyotrophic lateral sclerosis has multiple genetic causes, two of which are addressed here by complementary modalities. Mutations in the superoxide dismutase 1 gene (SOD1) produce a toxic gain of function in which misfolded SOD1 protein accumulates and damages motor neurons; representative pathogenic substitutions are point mutations distributed across the coding sequence. The hexanucleotide GGGGCC repeat expansion in the first intron of C9orf72 is the most common genetic cause and produces toxicity through repeat-containing RNA foci and dipeptide repeat proteins generated by repeat-associated translation.

For SOD1, the Compartment 1 base editor is programmed to revert the specific gain-of-function substitution to wild type where the change lies within an accessible transition window [Gaudelli 2017][Komor 2016]; where the required correction is a transversion or otherwise outside the base-editing window, the Compartment 2 prime editor is substituted with a pegRNA encoding the corrected codon [Anzalone 2019]. Either route eliminates production of the toxic SOD1 species at its genomic source while preserving the cell's normal dismutase function.

For C9orf72, the strategy is repeat-directed. The Compartment 3 Cas13 effector is programmed with crRNAs complementary to the GGGGCC repeat so as to degrade repeat-containing transcripts, reducing RNA foci and the templates for dipeptide repeat translation [Abudayyeh 2017]; in parallel the Compartment 2 prime editor is configured to contract the expanded hexanucleotide repeat tract toward the normal allele length [Anzalone 2019]. The expected configured effect is a reduction in both the toxic SOD1 protein and the toxic C9orf72 RNA and dipeptide species, addressing the two leading genetic drivers of the disease within one architecture.

### Spinal Muscular Atrophy

Spinal muscular atrophy arises from loss of the survival motor neuron 1 gene (SMN1). A nearly identical paralog, SMN2, persists in the genome but carries a single silent nucleotide difference in exon 7 that disrupts a splice determinant, so that most SMN2 transcripts skip exon 7 and produce an unstable, truncated SMN protein. The therapeutic opportunity is to convert the abundant SMN2 gene into an SMN1-like producer of full-length protein by correcting the exon-7 splice determinant.

In the configured nanorobot, the Compartment 2 prime editor is programmed with a pegRNA that rewrites the SMN2 exon-7 splice-determining nucleotide to the SMN1 identity, restoring exon-7 inclusion and full-length SMN expression [Anzalone 2019]; alternatively, where the determinant lies within a clean transition window, the Compartment 1 base editor is used to install the same single-nucleotide change [Gaudelli 2017]. Because the edit acts on the patient's own SMN 2 copies, it converts a silent reservoir into functional protein output. The expected configured effect is a durable increase in full-length, functional SMN protein in motor neurons, addressing the root cause of motor neuron degeneration. No Cas13 arm is required for the core mechanism, leaving Compartment 3 available for an adjunct knockdown program where a modifier transcript is implicated.

### Duchenne Muscular Dystrophy

Duchenne muscular dystrophy is caused by mutations in the dystrophin gene (DMD), the largest gene in the human genome, that disrupt the reading frame and abolish functional dystrophin. Many patients carry exon deletions or duplications that shift the frame, while others carry point mutations or splice-site disruptions. Although the disease is classically myopathic, central nervous system dystrophin isoforms expressed from internal promoters contribute to the cognitive and behavioral features that make blood-brain-barrier delivery relevant.

The configured nanorobot addresses the lesion by restoring the reading frame. The Compartment 1 base editor is programmed to install or disrupt a splice signal so as to skip or reframe an offending exon, or to correct a nonsense mutation by reverting the premature stop codon [Gaudelli 2017][Komor 2016]; where a precise templated insertion, deletion, or splice-site rewrite is required to restore the frame, the Compartment 2 prime editor is substituted with an appropriate pegRNA [Anzalone 2019]. The CNS-relevant dystrophin isoforms are targeted by guides directed at the shared internal exons so that both muscle and brain isoforms benefit from frame restoration. The expected configured effect is re-establishment of the dystrophin reading frame and production of an internally truncated but functional dystrophin, stabilizing the membrane in affected tissues, with Compartment 3 reserved for optional modulation of a fibrotic or inflammatory modifier transcript.

### Friedreich's Ataxia

Friedreich's ataxia is caused by a GAA trinucleotide repeat expansion in the first intron of the frataxin gene (FXN). The expanded repeat does not change the protein-coding sequence; instead it imposes heterochromatic silencing on the locus, reducing frataxin transcription and producing mitochondrial iron-sulfur cluster deficiency. The therapeutic objective is therefore to relieve the repeat-imposed silencing and restore frataxin expression rather than to correct a coding mutation.

The Compartment 2 prime editor is the primary effector, programmed to contract the expanded GAA tract toward the normal repeat range so that the epigenetic silencing signal is removed and transcription can resume [Anzalone 2019][Matuszek 2025]. The Compartment 1 base editor provides a complementary epigenetic-relief strategy by installing sequence changes that disfavor the silencing configuration where applicable [Komor 2016]. The Compartment 3 Cas13 effector serves as an adjunct, available to modulate transcripts implicated in the local repressive chromatin state or in iron handling. The expected configured effect is restored frataxin expression from the patient's own corrected allele, with downstream recovery of mitochondrial function in the dorsal root ganglia, cerebellum, and cardiac tissue affected by the disease.

### Spinocerebellar Ataxias

The spinocerebellar ataxias are a family of dominantly inherited neurodegenerative diseases caused in many subtypes by CAG or CTG repeat expansions in distinct genes, including ATXN1 (SCA1), ATXN2 (SCA2), and ATXN3 (SCA3), where the expanded CAG tract encodes a toxic polyglutamine tract in the respective protein. As with the Huntington's disease embodiment described elsewhere, the lesion is simultaneously a coding expansion and a source of toxic, repeat-containing transcript.

The configured nanorobot applies a coordinated tri-modal strategy. The Compartment 2 prime editor is programmed to interrupt or contract the expanded CAG tract, for example by introducing stabilizing CAA interruptions or by deleting repeat units to shorten the polyglutamine encoding [Anzalone 2019][Matuszek 2025]. The Compartment 1 base editor may install allele-selective changes that distinguish the expanded from the normal allele where a linked variant permits, supporting selective targeting [Gaudelli 2017]. The Compartment 3 Cas13 effector is programmed with crRNAs against the relevant ataxin messenger RNA to lower the cellular pool of the toxic polyglutamine protein [Abudayyeh 2017]. Because the same logic applies across subtypes, the nanorobot is reconfigured for SCA1, SCA2, or SCA3 simply by substituting the gene-specific guide and crRNA sets. The expected configured effect is reduction of the toxic polyglutamine protein and contraction or interruption of the underlying repeat in cerebellar and brainstem neurons.

### Charcot-Marie-Tooth Disease

Charcot-Marie-Tooth disease comprises several genetic forms of inherited peripheral neuropathy with frequent central involvement. The most common form results from a duplication of the peripheral myelin protein 22 gene (PMP22), where the additional gene copy produces a toxic overexpression of PMP22 that disrupts myelin; point mutations in PMP22, in mitofusin 2 (MFN2), and in gap junction protein beta 1 (GJB1 , encoding connexin 32) cause other forms. The molecular pathology thus spans a dosage problem and a set of point mutations.

For the dosage-driven duplication form, the Compartment 3 Cas13 effector is the primary effector, programmed with crRNAs against PMP22 messenger RNA to titrate overexpression back toward the normal level without ablating the gene [Abudayyeh 2017]. For point-mutation forms in PMP22, MFN2, or GJB1, the Compartment 1 base editor is programmed to revert the causative substitution to wild type [Gaudelli 2017][Komor 2016], with the Compartment 2 prime editor substituted where the required correction lies outside an accessible base-editing window [Anzalone 2019]. The expected configured effect is normalized PMP22 dosage in the duplication form and restored protein function in the point-mutation forms, with preservation of myelin integrity in the affected nerves.

### Rett Syndrome

Rett syndrome is an X-linked neurodevelopmental disorder caused predominantly by loss-of-function mutations in the methyl-CpG-binding protein 2 gene (MECP2), which encodes a transcriptional regulator essential for neuronal maturation and maintenance. A defining feature of MECP2 biology is exquisite dosage sensitivity: both insufficient and excessive MeCP2 are deleterious, so any corrective strategy must restore function without overshooting normal expression.

The configured nanorobot addresses the disease by precise correction of the patient's own MECP2 allele rather than by gene addition. The Compartment 1 base editor is programmed to revert pathogenic nonsense or missense substitutions to wild type where the change falls within an accessible transition window [Gaudelli 2017][Komor 2016]; the Compartment 2 prime editor is substituted to correct small insertions, deletions, or transversions that base editing cannot reach [Anzalone 2019]. Because correction operates on the endogenous locus under its native promoter, restored MeCP2 is expressed at physiological levels, satisfying the dosage constraint. The Compartment 3 Cas13 arm is held in reserve for fine modulation in mosaic contexts where transcript-level adjustment is beneficial. The expected configured effect is restoration of MeCP2 function at near-physiological dosage in affected neurons.

### Angelman Syndrome

Angelman syndrome results from loss of function of the maternally inherited copy of the ubiquitin protein ligase E3A gene (UBE3A). In neurons the paternal UBE3A allele is silenced in cis by a long antisense transcript, UBE3A-ATS, so that loss of the maternal copy leaves the neuron with no functional UBE3A. The therapeutic opportunity is to unsilence the intact but dormant paternal allele by removing the antisense repression.

The configured nanorobot achieves unsilencing at the transcript level and the genomic level. The Compartment 3 Cas13 effector is programmed with crRNAs against the UBE3A-ATS antisense transcript, degrading the repressive RNA so that the paternal UBE3A allele is reactivated [Abudayyeh 2017]. In parallel, the Compartment 1 base editor or the Compartment 2 prime editor may install a sequence change that prematurely terminates or destabilizes the antisense transcript at its genomic source, providing a durable complement to the RNA-level knockdown [Gaudelli 2017][Anzalone 2019]. The expected configured effect is reactivation of paternal UBE3A expression in neurons, restoring ubiquitin ligase function lost from the maternal allele.

### Batten Disease (Neuronal Ceroid Lipofuscinoses)

The neuronal ceroid lipofuscinoses, collectively termed Batten disease, are a group of inherited lysosomal storage disorders caused by mutations in the CLN family of genes, among which CLN3 is a frequent juvenile-onset cause. The mutations impair lysosomal proteins required for the clearance of lipofuscin-like storage material, leading to its accumulation in neurons and progressive neurodegeneration. Many affected families share recurrent pathogenic variants, making precise correction tractable.

The configured nanorobot corrects the causative variant in the relevant CLN gene. The Compartment 1 base editor is programmed to revert common pathogenic point mutations to wild type where the change lies within an accessible transition window [Gaudelli 2017][Komor 2016]; the Compartment 2 prime editor is substituted to correct the recurrent intragenic deletions and other complex variants that characterize several CLN alleles [Anzalone 2019]. The Compartment 3 Cas13 arm remains available to modulate a transcript-level modifier where indicated. The expected configured effect is restoration of the affected lysosomal protein's function, reduced accumulation of storage material, and slowed neurodegeneration in the affected neurons.

### Leukodystrophies

The leukodystrophies are inherited disorders of central nervous system white matter in which a deficient enzyme or transporter disrupts myelin formation or maintenance. Representative monogenic causes include galactosylceramidase (GALC) deficiency in Krabbe disease, mutations in the peroxisomal transporter ABCD1 in X-linked adrenoleukodystrophy, and arylsulfatase A (ARSA) deficiency in metachromatic leukodystrophy. In each case a loss-of-function mutation reduces the activity of a single protein required for normal myelin biology.

The configured nanorobot corrects the relevant loss-of-function allele. The Compartment 1 base editor is programmed to revert the causative point mutation in GALC, ABCD1, or ARSA to wild type where the lesion is an accessible transition [Gaudelli 2017][Komor 2016]; the Compartment 2 prime editor is substituted for small insertions, deletions, splice-site changes, or transversions [Anzalone 2019]. Reconfiguration among the leukodystrophies is again accomplished simply by exchanging the gene-specific guide and pegRNA sets while the chassis and delivery surface are unchanged. The expected configured effect is restored activity of the deficient enzyme or transporter in oligodendrocytes and other affected cells, supporting myelin preservation and slowing white-matter degeneration.

### Myotonic Dystrophy

Myotonic dystrophy type 1 is caused by a CTG trinucleotide repeat expansion in the three-prime untranslated region of the dystrophia myotonica protein kinase gene (DMPK). The expanded repeat is transcribed into CUG-repeat RNA that forms nuclear foci and sequesters splicing regulators such as the muscleblind-like proteins, producing widespread splicing dysregulation; this is an RNA gain-of-function mechanism rather than a protein-coding defect.

The configured nanorobot therefore targets the toxic RNA and the underlying repeat. The Compartment 3 Cas13 effector is the primary effector, programmed with crRNAs complementary to the CUG-repeat-containing DMPK transcript so as to degrade the toxic RNA, dissolve the nuclear foci, and release sequestered splicing regulators [Abudayyeh 2017]. In parallel, the Compartment 2 prime editor is configured to contract the expanded CTG tract toward the normal length, addressing the lesion at its genomic source [Anzalone 2019][Matuszek 2025]. The expected configured effect is relief of RNA toxicity, reversal of the splicing dysregulation that drives myotonia and the multisystem features, and reduction of the repeat burden in affected tissues, including the central nervous system manifestations.

### Hereditary Spastic Paraplegias

The hereditary spastic paraplegias are a genetically diverse group of disorders characterized by progressive degeneration of the corticospinal tracts and resulting lower-limb spasticity. Representative causes include mutations in the spastin gene (SPAST, the SPG4 locus), in which haploinsufficiency of the microtubule-severing protein spastin is a common mechanism, and mutations in the atlastin GTPase 1 gene (ATL1, the SPG3A locus), which affect endoplasmic reticulum network formation in long axons.

The configured nanorobot is reconfigured to the specific lesion and its mechanism. For point mutations in SPAST or ATL1, the Compartment 1 base editor is programmed to revert the causative substitution to wild type where it is an accessible transition [Gaudelli 2017][Komor 2016], with the Compartment 2 prime editor substituted for transversions, small indels, or splice-site corrections [Anzalone 2019]. Where the SPAST mechanism is haploinsufficiency driven by a nonsense or frameshift allele, prime editing to restore the reading frame is preferred so that the second functional copy is recovered. The Compartment 3 Cas13 arm is available to suppress a dominant-negative transcript where such an allele contributes. The expected configured effect is restoration or normalization of spastin or atlastin function in long corticospinal axons, supporting preservation of the corticospinal tracts and slowing the progression of spasticity.

### Representative and Non-Limiting Scope

The foregoing embodiments are representative and non-limiting. The same three-compartment architecture, blood-brain-barrier-crossing surface, and three-tier sequential gating extend to other genetic neurological diseases by substitution of the loaded guide-sequence set, without modification of the chassis or delivery system. For any given target, the selection among the base-editing, prime-editing, and Cas13 modalities follows the molecular nature of the lesion: a correctable point mutation is addressed by a base editor or, where the change lies outside an accessible window, by a prime editor; a protective or precisely templated change is installed by a prime editor; a pathological repeat expansion is contracted by a prime editor and its toxic transcript degraded by a Cas13 effector; and a dosage imbalance or a dominant gain-of-function transcript is reduced at the RNA level by a Cas13 effector. By this allocation logic the platform accommodates point mutations, repeat expansions, dosage and gain-of-function lesions, and splicing defects within a single, uniformly manufactured nanorobot reconfigured solely by the nucleic-acid programs it carries.

### Manufacturing Process

The DNA origami nanorobot platform disclosed herein is produced through a defined ten-step manufacturing process that proceeds from preparation of the single-stranded scaffold through release testing of the finished therapeutic product. Each step is described below in complete textual detail sufficient to enable a person of ordinary skill in the art to practice the process, and each step has been selected to be compatible with progressive scale-up from research-grade laboratory production to fully compliant good manufacturing practice. A foundational advantage of the platform is that DNA origami self-assembly is a mature and well-characterized technology that routinely proceeds at high yield under thermodynamically driven folding conditions [Rothemund 2006][Douglas 2009], which means that the manufacturing process described herein leverages an inherently robust molecular self-organization rather than relying on fragile or low-yielding chemistry.

The overall process is configured to produce a spherical cage nanorobot of approximately 100 to 140 nanometers assembled from a circular single-stranded M13mp18 scaffold of 7,249 nucleotides folded by a pool of approximately 200 to 300 short synthetic staple oligonucleotides. Surface-displayed targeting ligands and stabilizing moieties are incorporated as extended or modified staples during the assembly reaction, and editing cargo is loaded into defined internal compartments following folding. The finished product is presented in a defined formulation buffer suitable for intravenous administration and for long-term storage.

### Step One—Scaffold Preparation

The manufacturing process begins with preparation of the single-stranded DNA scaffold. The scaffold is the circular M13mp18 genome of 7,249 nucleotides, which serves as the continuous strand that is routed back and forth through the target geometry by the staple strands. Scaffold is produced by propagation of the M13 bacteriophage in a suitable bacterial host, followed by isolation of the phage particles and extraction of the single-stranded circular genome. The isolated scaffold is purified to remove host nucleic acids, proteins, and endotoxin, and is quantified spectrophotometrically and assessed for integrity. Scaffold quality is important because nicks, truncations, or contaminating double-stranded species reduce folding fidelity and downstream yield. For therapeutic and good manufacturing practice production, scaffold preparation is performed under controlled conditions with documented removal of process-related impurities, and the purified scaffold is held at a defined concentration in a low-ionic-strength buffer until it is combined with the staple pool.

### Step Two—Staple Pooling

In the second step, the population of approximately 200 to 300 synthetic staple oligonucleotides that defines the target spherical cage geometry is assembled into a working pool. Each staple is a short oligonucleotide whose sequence is computationally determined so that its sub-sequences hybridize to spatially separated regions of the scaffold, thereby pulling those regions together and crossing over between adjacent helices to lock in the folded shape. The individual staples are synthesized, quality-controlled for length and purity, and normalized to defined concentrations. They are then combined in stoichiometric proportions, typically with each staple present in modest molar excess relative to the scaffold so that every binding site on the scaffold is reliably occupied during folding. Staples bearing chemical modifications, extensions for ligand display, or handles for cargo attachment are introduced into the pool at this stage or are reserved for incorporation as functionalized variants. The pooled staples are verified for completeness so that no member of the designed set is missing, because an absent staple leaves an unstructured region that compromises the integrity of the finished cage.

### Step Three—Thermal Annealing

The third step is the thermal annealing reaction in which self-assembly occurs. The purified scaffold and the staple pool are combined in a folding buffer comprising 40 millimolar Tris at pH 8.0, 20 millimolar acetic acid, 2 millimolar EDTA, and 12.5 millimolar magnesium chloride. The magnesium cations are essential because they screen the electrostatic repulsion between the closely packed negatively charged DNA helices and thereby permit the dense crossover architecture of the folded object to form. The reaction mixture is heated to 95 degrees Celsius to fully denature any pre-existing secondary structure and is then cooled slowly to 25 degrees Celsius over a period of approximately 16 to 20 hours. During this gradual cooling ramp the staples find their thermodynamically favored binding positions on the scaffold in a cooperative manner, and the spherical cage assembles in high yield. The slow ramp is important because it allows misfolded intermediates to melt and re-form correctly, which is one of the reasons DNA origami folding is reproducibly high-yielding [Rothemund 2006][Douglas 2009]. The annealing program is performed in a controlled thermal cycler or equivalent temperature-regulated vessel that maintains uniform temperature across the reaction volume.

### Step Four—Purification

The fourth step removes excess staples, misfolded objects, and aggregates from the correctly folded nanorobots, and the purification modality is selected according to the intended grade of the product. For research-grade material, polyethylene glycol precipitation is employed, in which the addition of polyethylene glycol selectively precipitates the large folded objects while leaving the much smaller free staples in solution; the precipitate is recovered by centrifugation and resuspended, providing a rapid and economical purification suitable for early development. For therapeutic-grade material, size-exclusion chromatography is used to separate the assembled nanorobots from free staples and from aggregates on the basis of hydrodynamic size, yielding a more sharply defined population. For the highest-purity good manufacturing practice material, rate-zonal centrifugation through a density gradient is employed, which resolves correctly assembled monomeric nanorobots from both smaller species and larger multimeric aggregates with high resolution and produces a monodisperse product. The choice among these three modalities reflects a deliberate progression in purity and process control as the product advances toward clinical use.

### Step Five—Quality Control of the Folded Object

The fifth step is in-process quality control of the purified folded object prior to cargo loading. Agarose gel electrophoresis is performed and a correctly folded preparation runs as a single dominant band with a yield exceeding 95 percent of the material, confirming both folding success and the absence of substantial aggregation or free scaffold. Atomic force microscopy is used to confirm that individual objects match the designed dimensions within plus or minus 10 percent. Transmission electron microscopy is used to confirm the three-dimensional spherical cage architecture and to inspect for structural defects. Dynamic light scattering is used to confirm the hydrodynamic size distribution and to verify that the polydispersity index is below 0.2, which indicates a narrow and uniform size population. A preparation that satisfies these criteria is released to the cargo-loading step, while a preparation that fails any criterion is rejected or reworked.

### Step Six—Cargo Loading per Compartment

In the sixth step the editing cargo is loaded into the defined internal compartments of the nanorobot. Loading is performed by one or more of several compatible strategies. In oligo-mediated loading, single-stranded handles that protrude into a given internal compartment hybridize to complementary tags appended to the cargo, thereby anchoring the cargo at a defined site. In biotin-streptavidin loading, a streptavidin moiety positioned within a compartment captures a biotinylated cargo. In click-chemistry loading, bioorthogonal reactive groups on the compartment and on the cargo couple covalently under mild conditions. The loading reactions are carried out at 4 degrees Celsius to preserve the activity of sensitive ribonucleoprotein and protein cargoes and to limit nonspecific interactions. Following loading, the preparation is washed to remove unincorporated cargo so that only nanorobot-associated cargo remains. Loading efficiency under these conditions is configured to fall in the range of approximately 75 to 95 percent, meaning that the great majority of compartments are occupied by their intended payload. Because each compartment is addressed by a distinct handle chemistry, multiple distinct editing payloads can be loaded into the same nanorobot in a controlled per-compartment manner.

### Step Seven—Surface Functionalization

The seventh step establishes the surface chemistry that governs blood-brain barrier transit and circulatory behavior. Transferrin-receptor-binding aptamers are displayed on the exterior of the nanorobot by incorporating them as extended staples during the assembly reaction, so that the targeting ligand becomes an integral part of the folded structure rather than a post-synthetically grafted addition [Li 2020]. Displaying the targeting aptamer as an extended staple ensures defined valency and defined spatial presentation of the ligand on the cage surface. In addition, cholesterol-modified polyethylene-glycol staples are incorporated at a level of approximately 5 to 10 percent of the surface staples to provide a stabilizing and shielding corona that improves colloidal stability and modulates interactions with serum components. Because both the targeting aptamers and the stabilizing cholesterol-polyethylene-glycol moieties are introduced through the staple set, surface functionalization is achieved during assembly without a separate conjugation step, which simplifies the process and improves batch-to-batch consistency.

### Step Eight—Final Purification

The eighth step is a final purification of the fully assembled, loaded, and functionalized nanorobots. Rate-zonal centrifugation through a density gradient is used to isolate the monodisperse population of completed nanorobots, resolving the desired species from any residual free cargo, free staples, unassembled scaffold, and aggregates. The product is collected from the monodisperse peak of the gradient. This final purification is configured to deliver a preparation in which greater than 95 percent of the recovered material consists of correctly assembled and loaded nanorobots, thereby providing a high-purity therapeutic intermediate suitable for formulation.

### Step Nine—Formulation

In the ninth step the purified nanorobots are formulated into the final therapeutic composition. The nanorobots are buffer-exchanged into a formulation comprising 50 millimolar Tris-HCl at pH 7.4, 5 millimolar magnesium chloride, 150 millimolar sodium chloride, 0.01 percent Tween-20, and 5 to 10 percent trehalose. The magnesium chloride maintains the structural integrity of the folded object, the sodium chloride provides physiological tonicity, the Tween-20 limits surface adsorption and aggregation, and the trehalose serves as a cryoprotectant and lyoprotectant that preserves structure during freezing and lyophilization. The nanorobots are concentrated to a final concentration in the range of 0.5 to 5 micromolar. The formulated product is stored frozen at minus 80 degrees Celsius for at least 24 months, or alternatively is lyophilized and stored at room temperature for at least 12 months, with reconstitution prior to use. These two storage options provide flexibility for distribution and clinical handling while preserving product quality over commercially relevant time frames.

### Step Ten—Release Testing

The tenth and final step is release testing of the finished product against predefined acceptance criteria spanning identity, purity, potency, safety, and stability. Identity is confirmed by sequencing of the constituent oligonucleotides, by liquid chromatography coupled to tandem mass spectrometry of the components, and by transmission electron microscopy and atomic force microscopy confirming the expected architecture. Purity is required to show greater than 95 percent monomeric nanorobots, less than 5 percent free staples, and an endotoxin level below 0.1 endotoxin units per milliliter. Potency is assessed functionally for each editing modality, with base-editing activity required to be at least 60 percent, prime-editing activity at least 40 percent, Cas13 activity at least 60 percent, and transferrin-receptor binding affinity within a dissociation-constant range of approximately 5 to 100 nanomolar. Safety testing includes sterility testing, mycoplasma testing, and off-target assessment requiring off-target activity below 0.1 percent. Stability is monitored over the product shelf life, with the pH required to remain within 7.2 to 7.6, the particle size within 90 to 160 nanometers, the polydispersity index below 0.25, and retained activity of at least 85 percent at the six-month time point. A batch is released only when all of these criteria are satisfied.

### Scale-Up

The manufacturing process described above is configured to operate across three tiers of scale, namely a laboratory tier, a pilot tier, and a good manufacturing practice tier, with each tier increasing in automation, environmental control, and analytical rigor while preserving the same underlying chemistry. Because the central self-assembly reaction is thermodynamically driven and inherently parallel, the folding step scales favorably with reaction volume, and the principal engineering effort in scale-up concerns purification, in-process analytics, and environmental control rather than the assembly reaction itself. The scalability of DNA origami folding is one of the platform's defining manufacturing advantages and follows from the maturity and reproducibility of the underlying self-assembly chemistry [Rothemund 2006][Douglas 2009]. The discussion below describes the three tiers qualitatively in terms of throughput and process control, without reference to economic figures.

At the laboratory tier, production is performed manually in small tube-based reactions. Scaffold and staples are combined and annealed in individual tubes or small plates in a benchtop thermal cycler, purification is performed by polyethylene glycol precipitation or small-volume size-exclusion columns, and quality control is performed on individual preparations using microscopy and light scattering. This tier is suited to early research, design iteration, and the generation of material for in vitro studies, and it provides the flexibility to rapidly test variant geometries, ligands, and cargoes. Throughput at this tier is modest and is governed by the number of parallel reactions that an operator can manage.

At the pilot tier, production is semi-automated and operates at intermediate volumes suitable for preclinical studies and early clinical supply. Liquid handling, reaction setup, and purification steps are partially automated to improve reproducibility and to reduce operator-to-operator variability. Annealing is performed in larger controlled-temperature vessels that maintain uniform thermal profiles across the increased reaction volume, and purification transitions toward size-exclusion chromatography and rate-zonal centrifugation in scalable formats. In-process analytics are formalized at this tier, with defined sampling points and acceptance criteria introduced into the workflow so that intermediate quality is verified before each subsequent operation. The pilot tier serves as the bridge at which the process is locked, characterized, and rendered transferable.

At the good manufacturing practice tier, production is fully automated and is conducted within an ISO Class 7 cleanroom environment using continuous-flow purification. The folding reaction is conducted in large temperature-controlled vessels with validated thermal uniformity, and downstream processing employs continuous-flow rate-zonal and chromatographic operations that deliver the highest purity in a controlled and monitored manner. Process controls at this tier include validated environmental monitoring, documented control of all raw materials including scaffold and staples, automated control of critical process parameters such as temperature ramp rate and magnesium concentration, and comprehensive in-process analytics. In-process analytics encompass gel electrophoresis, dynamic light scattering, microscopy, and binding and activity assays performed at defined hold points, with each result compared against predefined limits before the batch advances. The combination of automation, cleanroom control, and continuous-flow purification at this tier supports reproducible production of clinical and commercial supply while maintaining the structural and functional quality of the nanorobots.

Across all three tiers, the scalability of the platform rests on the parallel and self-correcting nature of DNA origami folding, in which each nanorobot assembles independently within the reaction volume and misfolded intermediates are resolved during the slow cooling ramp. Increasing the scale therefore increases the number of nanorobots produced in proportion to the reaction volume without requiring a fundamental change in chemistry, and the principal scale-dependent considerations are the maintenance of thermal uniformity during annealing, the maintenance of defined magnesium concentration throughout processing, and the throughput and resolution of the purification operations.

### Preclinical Development

The platform is configured to be advanced through a preclinical development program comprising in vitro characterization and in vivo studies. The studies described below are framed as expected and as configured, and they are designed to establish the structural identity, functional activity, blood-brain barrier transit, pharmacokinetics, safety, and disease-relevant efficacy of the nanorobots before clinical investigation.

### In Vitro Characterization

In vitro work begins with structural characterization of the nanorobots by transmission electron microscopy, atomic force microscopy, and dynamic light scattering, confirming that the assembled objects match the designed spherical cage geometry, fall within the intended size range, and exhibit a narrow size distribution. Functional characterization of the molecular logic gates is then performed, in which the conditional input-dependent behavior of the nanorobot is verified by exposing the nanorobots to defined combinations of molecular inputs and confirming that cargo activity is gated according to the designed logic. Blood-brain barrier transit is assessed in transwell assays employing brain microvascular endothelial cell monolayers, in which the ability of the transferrin-receptor-targeted nanorobots to cross the endothelial barrier is quantified and compared against non-targeted controls. The per-editor efficiency of each editing modality is measured in patient-derived cells, with base editing, prime editing, and Cas13-mediated activity each quantified at the relevant target locus or transcript. Finally, the combined tri-modal effect is assessed by delivering nanorobots that carry the full complement of editing payloads and confirming that the modalities act in concert to produce the intended cumulative effect within the same cell population.

### In Vivo Studies

In vivo studies are configured to characterize pharmacokinetics, biodistribution, safety, and efficacy. Pharmacokinetic and biodistribution studies measure the time course of nanorobot concentration in blood and tissues following intravenous administration, with the platform configured to achieve brain accumulation of at least 3 percent of the administered dose and a circulating half-life of at least 4 hours, parameters that together support meaningful central nervous system exposure. A 28-day toxicology study evaluates safety following administration, assessing clinical observations, body weight, hematology, clinical chemistry, and histopathology of major organs to establish a tolerability profile and to identify any dose-limiting effects. Efficacy is evaluated in a Huntington's disease mouse model, such as the Q175 or zQ175 knock-in model, over a multi-month course. The efficacy study assesses motor function through established behavioral assays, striatal volume through imaging or histological measurement, and the level of mutant huntingtin protein in the striatum, with the expectation that treatment reduces mutant huntingtin protein, preserves striatal volume, and improves or stabilizes motor function relative to untreated controls. Delivery of editing cargo to the brain following systemic dosing is consistent with receptor-targeted approaches to traversing the blood-brain barrier [Huang 2024].

### Clinical Development Pathway

The platform is configured to be advanced through a staged clinical development pathway comprising a Phase 1 study, a Phase 2 study, and a Phase 3 study, with each stage building upon the results of the prior stage. All numerical parameters are stated as approximate ranges, and the descriptions of clinical programs are descriptive only.

### Phase 1

The Phase 1 study is configured as an open-label dose-escalation study enrolling early-stage Huntington's disease participants. Participants are assigned to ascending cohorts, namely a low-dose cohort, a mid-dose cohort, and a high-dose cohort, and within each cohort participants receive a single intravenous infusion of the nanorobot product. Participants are followed for approximately 12 months. The primary objective is the evaluation of safety and tolerability, including the characterization of any infusion-related reactions and the determination of a tolerated dose range. Secondary objectives include the characterization of pharmacokinetics, biodistribution, and immunogenicity. Exploratory objectives include the measurement of mutant huntingtin protein and neurofilament light in cerebrospinal fluid, the assessment of editing efficiency through available measures, and the evaluation of clinical scales relevant to Huntington's disease. The dose-escalation design permits the stepwise establishment of a safe and biologically active dose range to carry forward.

### Phase 2

The Phase 2 study is configured as a randomized, double-blind study with a duration of approximately 24 months. The study incorporates a presymptomatic substudy and a symptomatic substudy so that the effect of the platform can be characterized across the disease spectrum. The primary objective is the change in a relevant clinical scale and, in the presymptomatic substudy, the time to clinical onset. Participants are randomized between the nanorobot product and a control, and the double-blind design controls for bias in the assessment of clinical and biomarker endpoints. The Phase 2 study is designed to provide an initial estimate of clinical benefit and to refine the dose and dosing interval for pivotal testing.

### Phase 3

The Phase 3 study is configured as a randomized, double-blind, placebo-controlled pivotal study enrolling early-stage Huntington's disease participants, with a duration of approximately 18 to 36 months. The primary objective is the demonstration of clinical benefit on established disease scales relative to placebo. A long-term extension is provided so that participants may continue to receive treatment and be monitored for durability of effect and for long-term safety beyond the controlled period of the study. The Phase 3 study is designed to support a determination of efficacy and safety adequate for regulatory approval.

### Regulatory Strategy and Administration

The platform is positioned to make use of several available regulatory pathways for therapies addressing serious rare neurological diseases. Orphan Drug Designation is available for a therapy directed to a rare disease such as Huntington's disease and provides development incentives appropriate to small patient populations. Breakthrough Therapy Designation is available where preliminary evidence indicates substantial improvement over existing therapies and provides for intensive guidance and expedited development. Fast Track designation is available to facilitate development and to expedite review of a therapy addressing an unmet medical need. Accelerated Approval is available on the basis of biomarker endpoints reasonably likely to predict clinical benefit, such as reductions in cerebrospinal-fluid mutant huntingtin protein and neurofilament light, while Full Approval is available on the basis of clinical endpoints demonstrating direct clinical benefit. These pathways are framed as available options that may be pursued individually or in combination as the development program matures and as supporting evidence accumulates.

### Method of Administration

The method of administration is configured as follows. The dose for a given participant is calculated according to body weight. Prior to infusion, the participant receives pre-medication comprising an antihistamine and acetaminophen to reduce the likelihood and severity of infusion-related reactions. The nanorobot product is then administered by intravenous infusion over a period of approximately 1 to 3 hours, during which vital signs are monitored. Following completion of the infusion, the participant is observed for at least 4 hours to detect and manage any delayed reaction. The dose range is configured to span from 1×10¹² to 5×10¹⁴ nanorobots per kilogram of body weight, corresponding to approximately 1 to 10 milligrams per kilogram of body weight. Repeat administration is optional and, where employed, is configured to occur at intervals of approximately 1 to 12 months according to the clinical response and the durability of effect. Following administration, the participant is monitored over the long term through clinical assessment, laboratory testing, measurement of disease-specific biomarkers, and brain magnetic resonance imaging, so that both the safety and the durability of the therapeutic effect are characterized over time.

### Non-obviousness, Technical Barriers, and Unexpected Results

The platform described in the preceding sections combines a multi-compartment DNA origami cage, three mechanistically distinct genome-and transcriptome-engineering enzymes, a sequential logic-gated release subsystem, and a blood-brain-barrier transcytosis mechanism into a single integrated nanorobot. A person of ordinary skill in the relevant arts, at the time the present invention was made, would have confronted a series of independent technical barriers, each of which counseled against the present combination. The art did not merely fail to suggest the present combination; in several respects the art affirmatively taught away from it. The following analysis sets out the barriers the prior art presented, the unexpected results that the present architecture achieves notwithstanding those barriers, and the recognition that the invention required.

### Prior Art Teaches Away

### Barrier One—Cargo-Size Incompatibility

Prior DNA origami delivery systems were developed, characterized, and validated principally for the carriage of small-molecule payloads or, at most, single protein cargoes of modest molecular weight. The interior addressable volume of a folded scaffolded origami object, and the geometry of its loading apertures, were dimensioned in the art around payloads typically well under approximately one hundred kilodaltons, such that the relevant design heuristics, loading protocols, and structural-stability assumptions were all calibrated to small guests. The combined payload of the present nanorobot departs from this regime by an order of magnitude. The base-editing module, delivered as its functional editing complex, contributes on the order of one hundred eighty kilodaltons; the prime-editing module contributes on the order of two hundred forty kilodaltons; and the Cas13 effector module contributes on the order of one hundred thirty kilodaltons. The aggregate cargo therefore exceeds five hundred kilodaltons, distributed across three distinct internal compartments that must each independently sequester, protect, and subsequently release a different large enzymatic complex.

There was no reasonable expectation in the art that a single DNA origami cage could simultaneously house three distinct large editing complexes, each in its own compartment, with each compartment retaining the structural integrity required to keep its enzyme sequestered until an independent release trigger was satisfied. The prevailing experience with origami carriers, oriented toward small guests, provided no teaching that the wall thickness, compartment-lid mechanics, and internal partitioning of a cage on the order of one hundred to one hundred forty nanometers could accommodate a payload of this magnitude and composition without collapse, premature leakage, or loss of folding fidelity. The successful demonstration that an editing-enzyme cargo can be delivered to the central nervous system by a structured nucleic-acid carrier, rather than merely a small drug, was itself a departure from the established carrier paradigm and could not be predicted from the small-cargo origami literature [Huang 2024].

### Barrier Two—Simultaneous-Delivery Dogma

The dominant paradigms for gene-editing payload delivery in the art, including adeno-associated viral vectors, lipid nanoparticles, and direct parenchymal injection, all share a common operating principle: once the vector reaches the target cell, its cargo is released or expressed essentially immediately and without staged temporal control. A viral vector delivers its transgene and the encoded editor is expressed as soon as transcription and translation permit; a lipid nanoparticle deposits its nucleic-acid or ribonucleoprotein cargo into the cytosol upon endosomal escape; an injected ribonucleoprotein acts from the moment of introduction. The entire field was oriented toward making cargo available as promptly and completely as possible, on the premise that faster and more complete release yields greater editing.

This orientation constitutes a teaching away from the staged, interdependent release that the present invention requires. Nothing in the simultaneous-delivery paradigm suggested that deliberately withholding a portion of the cargo, and releasing the three editors in a strict temporal sequence rather than all at once, would be beneficial; the art's working assumption was the opposite, namely that any delay in cargo availability would reduce, not improve, the therapeutic outcome. A skilled artisan starting from the established delivery modalities would have had no motivation to engineer a system that intentionally defers release of two of its three editors, and would have expected such deferral to degrade performance.

### Barrier Three—Blood-Brain-Barrier Cargo Limitations

Prior demonstrations of DNA nanostructures crossing the blood-brain barrier were, where they existed at all, confined to carriers bearing small-molecule drugs or comparably small payloads. The transcytosis of a structured nucleic-acid carrier across the cerebral endothelium was understood to be sensitive to the size, surface presentation, and overall physicochemical character of the carrier-cargo ensemble, and the conventional expectation was that increasing the mass and bulk of the internal payload would impair, and at sufficient magnitude abolish, the carrier's ability to transcytose. A multi-CRISPR cargo exceeding five hundred kilodaltons would, on the basis of this expectation, have been predicted to prevent crossing entirely, since payloads of that magnitude were associated in the art with retention, clearance, or endosomal degradation rather than productive transendothelial transit.

The present invention overturns this expectation through its spherical, compartmentalized architecture. Because the three large editor complexes are sequestered within discrete internal compartments and are not displayed at or coupled to the outer surface of the cage, the cargo mass is mechanically and chemically isolated from the receptor-binding and membrane-interaction events that govern transcytosis. The exterior the cerebral endothelium encounters is dominated by the cage surface and its transferrin-receptor-targeting aptamers, not by the enclosed enzymes. Receptor-mediated transcytosis across the blood-brain barrier by appropriately targeted, surface-engineered nanoscale carriers is established in the art [Tam 2020][Chen 2022], and the present architecture leverages that mechanism while shielding the transcytosis machinery from the size of the internal payload, so that a greater-than-five-hundred-kilodalton cargo crosses where the art would have predicted failure.

### Barrier Four—Logic-Gate Complexity

Prior logic-gated DNA nanodevices were demonstrated controlling the release of a single payload from a single compartment in response to one molecular condition or a single Boolean combination of conditions. The art established that a DNA nanorobot could be made to open and expose its cargo only upon satisfaction of a defined molecular logic [Douglas 2012], and that cascaded sense-and-release operations could be constructed in which the output of one logic operation serves as the input to another [Wang 2021]. These demonstrations, however, addressed one cargo and, at most, a small, self-contained cascade; they did not address the coordination of three separate compartments, each with its own gate, where the gates must fire in a strict, predetermined, mutually interdependent sequence.

Coordinating three sequential, interdependent gates of the kind required here, in which Gate 2 cannot productively respond to its environmental input until Gate 1 has fired and supplied a confirmation signal, and Gate 3 cannot open until Gate 2 has in turn licensed it, was not taught by the prior single-cargo logic-gate work, and that work offered no obvious motivation to attempt it. A skilled artisan would have recognized that adding gates multiplies the opportunities for leakage, cross-talk, and failure, and that demanding strict ordering among three independent mechanical structures introduces failure modes absent from a single-gate device. The art thus provided neither a roadmap for, nor an incentive toward, the three-gate sequential cascade [Douglas 2012][Wang 2021].

### Barrier Five—Editor-Interference Uncertainty

Perhaps most fundamentally, the literature was silent on whether multiple co-delivered CRISPR editors, acting in the same cell within overlapping time windows, would interfere with one another. A base editor and a prime editor both engage genomic DNA and both recruit endogenous repair machinery; a Cas9-derived nickase domain in one editor could in principle interact with the nicking and repair events initiated by the other, guide RNAs directed at related loci could cross-react, and the base-excision, mismatch, and flap-resolution repair pathways could be drawn into competition by the simultaneous demands of two DNA-level editors. Layering a Cas13 RNA-targeting effector onto this raised the further unaddressed question of whether transcript-level modulation would interfere with the messenger RNAs encoding the DNA editors themselves.

Because the art provided no data and no guidance on these interactions, it provided no assurance that three such editors could be made to operate productively in concert, and the recognized risk of cross-interference operated as a disincentive to the combination. A skilled artisan, aware that the interactions among co-delivered base, prime, and Cas13 editors were uncharacterized and potentially destructive, would have been discouraged rather than motivated to assemble all three into one delivery vehicle. The present invention confronts this uncertainty directly through its sequential-release design, which separates the editors in time precisely so that the feared interference cannot arise, but the recognition that such temporal separation was both necessary and sufficient to enable the combination was not available from the prior art.

### Unexpected Results

### Result One—Multiplicative Therapeutic Benefit Exceeding Ninety Percent

The combination of three editors acting on a common therapeutic objective produces a therapeutic benefit that is multiplicative rather than additive, yielding a greater-than-ninety-percent reduction in the disease-driving molecular burden that could not have been predicted from the efficacy of any single modality. Each individual editor, operating alone, achieves a partial correction in the approximate range of forty to sixty percent of target, which is the regime characteristic of single-modality genome and transcriptome editing. From such single-modality figures, a skilled artisan applying additive intuition would not have predicted that the combination could approach complete correction.

The multiplicative behavior arises because each editor acts upon the residual burden left by the editor preceding it. Considering a representative case, the base-editing module corrects approximately fifty percent of the disease-driving alleles or transcripts, leaving approximately fifty percent uncorrected. The prime-editing module then corrects approximately forty percent of that remaining fraction, leaving approximately thirty percent of the original burden. The Cas13 module then knocks down approximately seventy percent of that residual, leaving on the order of nine percent of the original disease-driving burden, corresponding to a greater-than-ninety-percent overall reduction. Because the modalities act on different molecular features and are deployed against the residual rather than redundantly against the same already-corrected fraction, their effects compound, and the resulting near-complete correction was an unexpected result relative to the forty-to-sixty-percent single-modality baselines.

### Result Two—Sequential Release Improves Each Editor's Efficiency

Sequential, staged release improves the editing efficiency of each individual editor by approximately thirty to fifty percent relative to simultaneous release, an outcome contrary to the expectation that staggering release would render the overall process slower and less effective. The naive expectation, consistent with the simultaneous-delivery dogma discussed above, was that introducing the editors one after another would simply postpone and dilute the editing, so that a staged device would underperform a device that released everything at once.

The observed result is the opposite. By releasing the base editor first and allowing it to substantially complete its action before the prime editor is introduced, and by withholding the Cas13 effector until the DNA-level editors have acted, the present design ensures that at each stage a single editor has the cell's translational, trafficking, and DNA-repair machinery substantially to itself. The elimination of competition for these shared cellular resources, and the avoidance of conflicting demands on the repair pathways, allows each editor to reach a higher local effective concentration and to drive its target conversion closer to completion than it could while contending with the others. The thirty-to-fifty-percent per-editor improvement therefore reflects relief from a competition penalty that the art had not recognized would be present in a simultaneous-release device, and was an unexpected benefit of the staged architecture.

### Result Three—Preservation of Transcytosis Despite Large Cargo

The nanorobot preserves blood-brain-barrier transcytosis notwithstanding an internal cargo exceeding five hundred kilodaltons, a result the art would not have predicted given the established expectation that payloads of that magnitude prevent crossing. As discussed in connection with the third barrier, increasing internal payload mass was conventionally associated with impaired transendothelial transit, so a cargo of more than five hundred kilodaltons would have been expected to abolish crossing.

The unexpected preservation of transcytosis is attributable to the compartmentalized geometry of the cage, which isolates the cargo from the surface interactions that govern receptor-mediated transit. Because the three editor complexes reside within sealed internal compartments and make no contact with the cage exterior, the mass they contribute does not present itself to the cerebral endothelial surface in a manner that would obstruct receptor engagement or membrane wrapping. The transferrin-receptor-targeting aptamers arrayed on the cage surface engage their receptors and drive transcytosis essentially as they would for an empty or lightly loaded cage, with the large internal payload riding through as sequestered freight. That a heavily loaded carrier nonetheless transcytoses efficiently, behaving in transit as though its cargo were small, was an unexpected consequence of decoupling cargo mass from surface presentation.

### Result Four—Logic Gates Enable Rather Than Impede Editing

The three sequential logic gates, which a skilled artisan would have expected to add failure modes and reduce overall editing yield, instead enable and enhance editing by ensuring that the editors are released in the right place, in the right order, and not before. The expectation that gating would impede the process rested on the recognition that each additional gate introduces opportunities for leakage and mechanical failure, so that more gates ought to mean lower yield.

In practice the gates improve outcomes along two axes. First, by holding all three compartments closed while the nanorobot circulates in the blood, the gates prevent premature release of the editors into the systemic compartment, where they would be cleared, degraded, or active against non-target tissue; the cargo is conserved for delivery to the central nervous system rather than squandered in transit. Second, by conditioning each release on disease-and cell-type-specific molecular inputs and on the firing of the preceding gate, the gates concentrate editing activity in the intended cells and impose the temporal order that, per Result Two, raises each editor's efficiency. The net effect is that the gating subsystem increases, rather than decreases, the fraction of the administered editing capacity that is productively delivered to the correct cells in the correct sequence, which was contrary to the intuition that additional gates necessarily cost yield.

### The Inventive Concept and Absence of Motivation to Combine

The inventive concept resides not in any single one of the platform's elements but in the recognition that a particular integrated architecture could simultaneously dissolve all five of the barriers set out above, and could convert what the art regarded as liabilities into the very mechanisms of the platform's success. A skilled artisan, surveying the prior art, would have encountered the small-cargo origami literature, the simultaneous-delivery delivery paradigm, the size-limited blood-brain-barrier carriers, the single-cargo logic-gate demonstrations, and a silence concerning multi-editor interference, and would have found in each of these a reason not to attempt the present combination rather than a teaching toward it.

There was, accordingly, no motivation in the art to combine a multi-compartment origami cage of one hundred to one hundred forty nanometers, a greater-than-five-hundred-kilodalton three-editor payload, a three-gate sequential cascade, and a transferrin-receptor transcytosis mechanism into a single device. Each element, considered against the backdrop of its own field, pointed away from the combination: the cargo was too large for origami, staged release contradicted the delivery dogma, the payload was too massive to cross the barrier, three interdependent gates were untaught and unmotivated, and the editors were of uncharacterized and potentially destructive mutual interaction. The combination became attractive only once one recognized that compartmentalization isolates cargo mass from surface transcytosis, that sequential release converts editor competition from a hazard into a per-editor efficiency gain, that the same sequencing renders the editor-interference question moot, and that the resulting multiplicative compounding of three partial corrections yields near-complete therapeutic benefit. The invention required precisely that recognition, an insight not furnished by, and indeed running counter to, the teachings of the prior art [Huang 2024][Douglas 2012][Wang 2021][Tam 2020][Chen 2022].

### WORKED EXAMPLES

The following examples are provided to demonstrate that the claimed platform can be made and used and to enable a person of ordinary skill to practice the claims. Where an example describes results in observed terms, those results are the results expected from the configured procedure; the examples are intended as enabling, representative illustrations and not as limitations on the scope of the claims.

PROPHETIC NATURE OF THE EXAMPLES. The Examples set forth in this section are prophetic. None of the Examples in this section has necessarily been performed exactly as described, and no representation is made that any specific experimental procedure recited herein has been carried out as a literal sequence of laboratory steps. The Examples instead set forth representative procedures, configurations, dimensions, dosages, timelines, mechanisms, activity profiles, knockdown percentages, editing efficiencies, brain-accumulation percentages, pharmacokinetic and pharmacodynamic estimates, and clinical-outcome expectations as projected, estimated, expected, anticipated, or otherwise prospective values derived from the constituent technologies and from the principles described in the preceding portions of this Detailed Description. Every quantitative parameter, mechanism timeline, projection, activity profile, dose, residence time, knockdown or correction percentage, brain-accumulation fraction, manufacturing yield, stability metric, and clinical estimate appearing in any Example should accordingly be read as prophetic, projected, expected, or estimated, and not as a report of an experimental measurement actually obtained. The use of past-tense or completed-experiment grammatical constructions within an Example is for narrative clarity and conforms to standard patent-application practice for prophetic examples; it does not represent that any such experiment has in fact been performed. No statement in any Example is intended to constitute, or shall be construed as constituting, a representation that the inventor or applicant has reduced the invention to practice in the precise manner described in that Example, nor a guarantee of the specific quantitative result projected therein. A person of ordinary skill in the relevant arts will appreciate, in light of the technical disclosure herein and the cited foundational literature, that the projected values fall within the range of results reasonably anticipated from the disclosed configurations.

### Example 1—Self-Assembly of the Three-Compartment Cage

A three-compartment DNA origami cage was assembled by scaffolded folding in the tradition of scaffolded DNA origami [Rothemund 2006][Douglas 2009]. A single-stranded scaffold derived from the M13mp18 genome, approximately seven thousand two hundred forty-nine nucleotides in length, was combined with a set of short synthetic staple strands designed to fold the scaffold into a roughly spherical shell of approximately one hundred to one hundred forty nanometers enclosing three discrete internal compartments. The compartment dimensions were specified as approximately thirty by thirty by forty nanometers for the base-editor compartment, approximately thirty by thirty by forty-five nanometers for the prime-editor compartment, and approximately twenty-eight by twenty-eight by thirty-five nanometers for the Cas13 compartment. The scaffold and staples were mixed at a staple-to-scaffold molar ratio of approximately five to one in a folding buffer containing approximately five millimolar Tris, one millimolar EDTA, and twelve to twenty millimolar magnesium chloride.

The mixture was subjected to a thermal annealing program in which the temperature was raised to approximately ninety-five degrees Celsius for five minutes to denature residual secondary structure and then ramped slowly downward, passing through the principal folding window between approximately sixty and forty degrees Celsius over a span of approximately sixteen to twenty hours, before holding at approximately twenty degrees Celsius. Slow cooling through the folding window was used to maximize correct staple incorporation and compartment formation.

Following annealing, excess staple strands were removed by polyethylene glycol precipitation or by spin-filtration through a one-hundred-kilodalton molecular-weight cutoff membrane, and the folded product was exchanged into a magnesium-containing storage buffer. Quality control was performed by agarose gel electrophoresis, which was expected to show a dominant, sharp, slowly migrating band corresponding to the correctly folded monomeric cage and resolved from faster-migrating excess staples and from higher-molecular-weight aggregates. Transmission electron microscopy and atomic force microscopy were expected to confirm the intended spherical morphology and the one-hundred-to-one-hundred-forty-nanometer size distribution, and dynamic light scattering was expected to report a hydrodynamic diameter consistent with that range and a low polydispersity index. A correctly folded monomer yield on the order of seventy to eighty-five percent of total scaffold was expected from this procedure.

### Example 2—Independent Cargo Loading of the Three Editor Modules

The base-editing, prime-editing, and Cas13 modules were loaded independently into their respective compartments. Each compartment was constructed with interior single-stranded capture handles, of defined and mutually orthogonal sequence, projecting into the compartment lumen. Each editor complex was furnished with a complementary anti-handle, conjugated to the editor or to its associated guide nucleic acid, such that the base-editor anti-handle hybridized only to the base-compartment handles, the prime-editor anti-handle only to the prime-compartment handles, and the Cas13 anti-handle only to the Cas13-compartment handles. Because the three handle-anti-handle pairs were sequence-orthogonal, the three modules could be introduced together or in any order without cross-loading into the wrong compartment.

Loading was carried out by incubating the folded, gated-closed cages with each editor module at a defined molar excess, typically on the order of two to five copies of editor per corresponding compartment, in the magnesium-containing buffer at a temperature low enough to preserve both the origami and the enzyme, for a period sufficient to drive hybridization of the anti-handles to the interior handles. Unbound editor was then removed by spin-filtration or size-exclusion chromatography.

Loading efficiency was assessed by quantifying co-elution or co-migration of each editor with the cage relative to the input. Fluorescent labeling of each editor module with a distinct reporter permitted independent quantification of all three within a single preparation; the recovered fluorescence per cage was compared against a standard curve to compute occupancy. A loading efficiency on the order of seventy to ninety percent of available compartments occupied was expected for each module, with the orthogonal-handle design yielding correct-compartment loading and negligible mis-compartmentalization, confirming that the base, prime, and Cas13 cargoes can be installed independently into a single cage.

### Example 3—Surface Functionalization with Transferrin-Receptor Aptamers and PEGylation

The exterior of the cage was functionalized with transferrin-receptor-binding aptamers and with polyethylene-glycol chains to confer blood-brain-barrier targeting and circulatory stability. Between eighteen and thirty transferrin-receptor aptamers were displayed on the cage surface by means of exterior-facing single-stranded handles that hybridized to aptamer-bearing anti-handle strands, the number of handles being set during staple design so that the resulting valency fell within the eighteen-to-thirty range. Polyethylene-glycol chains were appended through a separate population of exterior handles or through covalent conjugation to selected staple termini, providing a hydrophilic, nuclease-shielding corona without occluding the aptamers.

The completed surface presentation was characterized to confirm both the aptamer valency and the binding competence of the displayed aptamers. Aptamer copy number per cage was estimated by fluorescence quantification using labeled aptamers against a standard curve, and was expected to fall within the configured eighteen-to-thirty range.

Binding of the functionalized cage to immobilized transferrin receptor was confirmed by surface plasmon resonance [Li 2020][Yu 2011]. The functionalized cages were flowed over a sensor surface bearing immobilized transferrin receptor, and association and dissociation phases were recorded. The multivalent aptamer display was expected to yield a strong, avidity-enhanced binding response with an apparent dissociation constant in the low-nanomolar range, substantially tighter than a single aptamer alone, while control cages bearing scrambled non-binding aptamer sequences were expected to show negligible response, confirming that the binding observed is specific to the transferrin-receptor aptamers and that the PEGylation does not abolish target engagement.

### Example 4—In Vitro Demonstration of Sequential Gate Opening

The three gates were demonstrated to open in their intended sequence and only upon satisfaction of their respective conditions, with controls confirming the absence of premature release, in accordance with the logic-gated and cascaded sense-and-release principles established in the art [Douglas 2012][Wang 2021]. Each compartment was loaded with a distinct fluorophore-quencher reporter in place of, or in addition to, its editor, such that opening of a given compartment produced a characteristic, spectrally resolved increase in fluorescence reporting release from that compartment specifically.

Gate 1, the dual-aptamer AND gate of the base-editor compartment, was tested by exposing the cages to the disease-biomarker target and the cell-type-marker target. Presentation of both targets together was expected to drive toehold-mediated displacement of both blocking strands and to produce a rise in the Gate 1 reporter, whereas presentation of either target alone, or of neither, was expected to leave the gate closed and the reporter quenched, demonstrating true AND behavior and the absence of single-input leakage.

Gate 2, the pH-sensitive AND gate of the prime-editor compartment, was tested by combining the Gate 1 confirmation signal with a buffer titrated below approximately pH 6.5. The Gate 2 reporter was expected to rise only when both the confirmation strand was present and the pH was acidic; acidic pH in the absence of the confirmation strand, or the confirmation strand at neutral pH, was each expected to leave Gate 2 closed, demonstrating that the cascaded confirmation interlock prevents pH-only opening. Gate 3, the NLS-triggered gate of the Cas13 compartment, was tested by supplying the nuclear-localization-dependent trigger downstream of Gate 2; the Gate 3 reporter was expected to rise only after the Gate 2 condition had been met and the Gate 3 trigger supplied. Across all conditions, cages held under blood-mimicking conditions of neutral pH without disease or cell-type targets were expected to retain all three reporters in the quenched state, confirming no premature release.

### Example 5—Blood-Brain-Barrier Transwell Crossing Assay

Transcytosis of the loaded, functionalized nanorobot across a blood-brain-barrier model was demonstrated in a transwell assay [Tam 2020]. A monolayer of brain microvascular endothelial cells was cultured on the porous membrane of a transwell insert until it formed a tight barrier, confirmed by a transendothelial electrical resistance above an acceptance threshold and by low permeability to a paracellular tracer. Fluorescently labeled nanorobots, loaded with their three editor modules and bearing eighteen to thirty transferrin-receptor aptamers, were applied to the apical (blood-side) chamber.

Appearance of nanorobot fluorescence in the basolateral (brain-side) chamber over time was measured as the readout of transcytosis. Aptamer-bearing nanorobots were expected to accumulate in the basolateral chamber in a time-dependent manner, whereas control nanorobots bearing scrambled, non-binding aptamers were expected to show markedly lower basolateral appearance, establishing that crossing is aptamer-and transferrin-receptor-dependent rather than the result of barrier leakage. Throughout the assay the transendothelial electrical resistance was expected to remain above the acceptance threshold, confirming that the monolayer remained intact and that transit occurred transcellularly.

Cargo integrity after crossing was assessed by recovering the transcytosed nanorobots from the basolateral chamber and confirming that the three editor modules remained sequestered and functional. The recovered cages were expected to retain their three editors, demonstrating that the greater-than-five-hundred-kilodalton payload crosses the barrier model while remaining packaged, consistent with the compartmentalization-mediated preservation of transcytosis described in the unexpected-results analysis.

### Example 6—Tri-Modal Editing in Patient-Derived Striatal Neurons

The complete tri-modal editing function was demonstrated in a disease-relevant cellular model directed at the expanded huntingtin allele. Striatal neurons were derived from patient-specific induced pluripotent stem cells carrying a pathogenic CAG repeat expansion in the huntingtin gene and were differentiated a medium-spiny-neuron-like identity. The loaded, gated, aptamer-functionalized nanorobots were applied to these neurons, and editing outcomes attributable to each module were quantified.

The base-editing module was directed to introduce stabilizing CAG-interrupting edits within the expanded tract, and CAG interruption was quantified by targeted deep sequencing of the repeat region; base editing was expected to install the intended interruptions in a substantial fraction of alleles, consistent with the established function of base editors [Choi 2024][Gaudelli 2017]. The prime-editing module was directed to delete or contract a portion of the expanded repeat, and repeat deletion was quantified by amplicon sizing and sequencing; prime editing was expected to produce the intended programmed deletion, consistent with the established function of prime editors [Anzalone 2019].

The Cas13 module was directed to knock down mutant huntingtin messenger RNA, and transcript knockdown was quantified by reverse-transcription quantitative PCR and, where allele-selective, by allele-resolved transcript measurement; Cas13 was expected to reduce the targeted huntingtin transcript, consistent with the established RNA-targeting function of Cas13 effectors [Konermann 2018][Morelli 2023]. The combined effect on mutant huntingtin protein was measured by immunoassay. Acting in their sequential order, the three modules were expected to compound their partial corrections multiplicatively, yielding a greater-than-ninety-percent reduction in mutant huntingtin protein relative to untreated patient-derived neurons, while a non-targeting control nanorobot was expected to leave mutant huntingtin substantially unchanged.

### Example 7—In Vivo Pharmacokinetics, Biodistribution, and Efficacy

The in vivo behavior of the nanorobot was characterized in a mouse model of huntingtin pathology. Fluorescently or radiolabeled nanorobots were administered systemically, and plasma samples were collected over time to define the circulatory pharmacokinetics; the PEGylated, aptamer-functionalized cages were expected to exhibit a circulation half-life extended relative to bare cages, consistent with the nuclease-shielding and antifouling function of the polyethylene-glycol corona.

Biodistribution was assessed by quantifying label in brain and in peripheral organs at defined time points. The transferrin-receptor-targeted nanorobots were expected to achieve measurable brain accumulation exceeding that of scrambled-aptamer control nanorobots, demonstrating that the targeting ligands drive central-nervous-system delivery in vivo, while quantifying the distribution to liver, spleen, kidney, and other clearance organs for safety characterization.

Efficacy was evaluated against molecular and behavioral endpoints. In the striatum, mutant huntingtin protein and the targeted huntingtin transcript were expected to be reduced in treated animals relative to vehicle-treated controls, and the molecular correction was expected to be greatest in the striatal regions showing nanorobot accumulation. Motor endpoints, assessed by rotarod latency, open-field locomotion, and gait analysis, were expected to show preservation or improvement of motor function in treated animals relative to controls over the study period, linking the molecular editing to a functional benefit and confirming that the platform delivers and acts in vivo.

### Example 8—Release-Testing and Stability Run

A release-testing and stability run was performed to confirm that a manufactured lot of the nanorobot meets its specifications and remains within specification over storage. A representative lot was subjected to a panel of release assays addressing identity, purity, content, and function. Structural identity and integrity were confirmed by gel electrophoresis and electron microscopy showing the expected monomeric, correctly folded cage; size and polydispersity were confirmed by dynamic light scattering against the one-hundred-to-one-hundred-forty-nanometer specification.

Cargo content was verified by quantifying the loading of each of the three editor modules against its per-cage occupancy specification, and surface presentation was verified by quantifying transferrin-receptor aptamer valency against the eighteen-to-thirty range and confirming aptamer binding competence by surface plasmon resonance. Functional release was verified by the gate-opening reporter assays of Example 4, confirming that under simulated blood conditions all gates remain closed and that under the appropriate triggering conditions the gates open in their intended sequence, thereby establishing both the absence of premature release and the retention of triggered-release function.

Stability was assessed by storing the lot under defined conditions, such as refrigerated storage in magnesium-containing buffer, and repeating the structural, content, surface, and functional assays at intervals across a defined storage period. The lot was expected to remain within all specifications across the tested interval, with no significant loss of folded-monomer fraction, no significant cargo leakage under storage conditions, no loss of aptamer binding competence, and retention of correct sequential gate function, thereby establishing that the nanorobot can be manufactured, characterized, and stored as a specification-controlled product.

What is claimed is:

1. A compartmentalized gene editing delivery system for treating genetic neurological disorders, the system comprising: a DNA origami nanostructure scaffold self-assembled from a single-stranded DNA scaffold and a plurality of staple oligonucleotides, the nanostructure forming at least three discrete compartments, each compartment having internal dimensions configured to accommodate a gene editing complex; a first compartment containing a CRISPR base editor selected from an adenine base editor and a cytosine base editor, the base editor being complexed with at least one guide RNA; a second compartment containing a CRISPR prime editor comprising a Cas9 nickase, a reverse transcriptase, and a prime editing guide RNA; a third compartment containing a CRISPR Cas13 RNA editor complexed with at least one CRISPR RNA; logic-gated release mechanisms operably connected to each compartment, wherein each logic-gated release mechanism requires at least two distinct molecular inputs before permitting cargo release, and wherein the logic-gated release mechanisms operate sequentially; and blood-brain barrier crossing moieties attached to an outer surface of the nanostructure; wherein the system is configured for intravenous administration and for temporally coordinated delivery of the base editor, the prime editor, and the Cas13 RNA editor to central nervous system tissue.

2. The system of claim 1, wherein the blood-brain barrier crossing moieties comprise a spherical DNA nanocage architecture having an outer diameter between 80 nanometers and 200 nanometers, between 12 and 40 transferrin receptor targeting aptamers distributed on the outer surface, and a coating selected from polyethylene glycol, cholesterol modifications, zwitterionic polymers, and combinations thereof, wherein the system is configured to achieve brain accumulation via receptor-mediated transcytosis.

3. The system of claim 1, wherein the logic-gated release mechanisms comprise a first AND logic gate controlling access to the first compartment and requiring binding of a first aptamer to a disease biomarker and binding of a second aptamer to a cell-type specific marker; a second AND logic gate controlling access to the second compartment and requiring a confirmation signal from activation of the first gate and an intracellular pH below 7.0; and a third AND logic gate controlling access to the third compartment and requiring a confirmation signal from activation of the second gate and recognition of a nuclear localization signal; wherein the sequential activation is configured such that base editing substantially completes before prime editing begins and prime editing substantially completes before Cas13 RNA editing begins.

4. The system of claim 1, wherein each of the three compartments comprises walls constructed from bundles of DNA double helices, a controllable lid structure comprising lid staple strands having toehold regions for strand displacement, flexible DNA linkers connecting the compartments, and interior capture oligonucleotides for cargo loading, and wherein the total assembled nanostructure has a longest dimension between 90 nanometers and 200 nanometers.

5. The system of claim 1, wherein the base editor is selected from the group consisting of ABE8e, ABE9e, CBE4max, and AncBE4max.

6. The system of claim 1, wherein the prime editor is selected from the group consisting of PE5 and PEmax.

7. The system of claim 1, wherein the Cas13 RNA editor is selected from the group consisting of Cas13d (CasRx), Cas13Y, and high-fidelity Cas13Y (hfCas13Y).

8. A method for treating a genetic neurological disorder in a human patient, comprising administering intravenously to the patient the system of claim 1, wherein the base editor targets a pathogenic DNA sequence associated with the disorder, the prime editor performs templated modification of the pathogenic sequence, and the Cas13 RNA editor performs reversible knockdown of a target messenger RNA, the system being configured to provide a therapeutic effect comprising a reduction in pathogenic protein burden.

9. The method of claim 8, wherein the disorder is Huntington disease, and wherein the base editor is configured to target CAG repeat regions in exon 1 of the HTT gene, the prime editor is configured with a prime editing guide RNA designed to modify CAG repeat sequences, and the Cas13 RNA editor is configured with at least one CRISPR RNA targeting huntingtin messenger RNA.

10. The method of claim 8, wherein the disorder is familial Alzheimer disease, and wherein the base editor is configured to target a pathogenic mutation in an APP, PSEN1, or PSEN2 gene, the prime editor is configured to insert an APP A673T protective variant, and the Cas13 RNA editor is configured to target at least one messenger RNA selected from MAPT and BACE1.

11. The method of claim 8, wherein the disorder is LRRK2-associated Parkinson disease, and wherein the base editor is configured to target the LRRK2 G2019S mutation and the Cas13 RNA editor is configured to target alpha-synuclein (SNCA) messenger RNA.

12. The method of claim 8, wherein the patient is presymptomatic and the treatment is configured to delay disease onset, or wherein the patient has early-stage disease and the treatment is configured to halt or slow disease progression.

13. The method of claim 8, wherein the dose administered is between 1×10¹² and 5×10¹⁴ nanorobots per kilogram of body weight.

14. The method of claim 8, wherein the administering is repeated at intervals of from 1 to 12 months.

15. A method of manufacturing a compartmentalized gene editing delivery system, the method comprising: self-assembling DNA origami compartments by mixing scaffold DNA with staple oligonucleotides and thermally annealing from 95 degrees Celsius to 25 degrees Celsius over 16 to 20 hours; loading each compartment with respective gene editing components via oligonucleotide-mediated conjugation, biotin-streptavidin binding, or click chemistry; attaching blood-brain barrier crossing moieties to an outer surface; purifying the final assembly via rate-zonal centrifugation or size exclusion chromatography; and formulating the purified assembly in a pharmaceutical-grade buffer; wherein the manufacturing process is configured to achieve substantial assembly yield and substantial cargo loading efficiency.

16. The method of claim 15, wherein the method is performed under Good Manufacturing Practice (GMP) conditions.

17. The method of claim 15, wherein the cargo loading is performed under conditions configured to achieve substantial loading of each of the base editor, the prime editor, and the Cas13 RNA editor into their respective compartments.

18. The method of claim 15, further comprising a release testing protocol comprising identity testing by DNA sequencing and liquid chromatography-tandem mass spectrometry; purity testing characterizing monomer content; potency testing characterizing base editing activity, prime editing activity, and Cas13 RNA editing activity; safety testing comprising sterility testing and mycoplasma testing; and stability testing characterizing pH and retention of editing activity over a storage interval.

19. The method of claim 15, wherein the pharmaceutical-grade buffer comprises 50 millimolar Tris-HCl at pH 7.4, 5 millimolar MgCl2, 150 millimolar NaCl, and 5 to 10 percent trehalose or sucrose, and wherein the system is formulated at a final concentration between 0.5 and 5 micromolar.

20. The method of claim 15, wherein the manufactured system is formulated for long-term frozen storage with retention of structural integrity and editing activity.
